(19) United States
(12) Patent Application Publication
(10) Pub. No.: US 2025/0195651 A1
(43) Pub. Date: Jun. 19, 2025

(54) USE OF ANTIGEN PRESENTING CELLS TO ENHANCE CAR-T CELL THERAPY

(71) Applicant: CELLEDIT LLC, Worcester, MA (US)

(72) Inventor: Biliang HU, Changsha, Hunan (CN)

(21) Appl. No.: 18/847,011

(22) PCT Filed: Mar. 14, 2023
(86) PCT No.: PCT/US2023/064291

§ 371 Date: Sep. 13, 2024

(30) Foreign Application Priority Data
Mar. 15, 2022 (WO) PCT/CN2022/080943

(51) Int. Cl. A61K 40/11; A61K 40/24; A61K 40/31; A61K 40/32; A61K 40/42; A61P 35/02; C07K 16/28; C07K 16/30; C12N 5/0783

(52) U.S. Cl. CPC A61K 40/11; A61K 40/24; A61K 40/31; A61K 40/32; A61K 40/4202; A61K 40/4205; A61K 40/4211; A61K 40/4255; A61K 40/4261; A61P 35/02; C07K 16/2803; C07K 16/2878; C07K 16/30; C12N 5/0636; A61K 2239/29; C07K 2317/31; C12N 2510/00

(57) ABSTRACT

Cancer therapy comprising both a population of genetically engineered T cells expressing a chimeric antigen receptor (CAR) and a population of antigen-presenting cells (APCs), which enhances efficacy of the CAR-expressing T cells.

### CROSS REFERENCE TO RELATED APPLICATIONS

This application claims the benefit of the filing dates of International Application No. PCT/CN2022/080943, filed Mar. 15, 2022, the entire contents of which are incorporated by reference herein.

### SEQUENCE LISTING

The instant application contains a Sequence Listing which has been filed electronically in XML format and is hereby incorporated by reference in its entirety. Said XML copy, created on Mar. 13, 2023, is named 112126-0046-70006WO01_SEQ.XML and is 196,714 bytes in size.

### BACKGROUND OF THE INVENTION

Adoptive cell transfer therapy is a type of immunotherapy that involves ex vivo expansion of immune cells, which may be modified to express a chimeric antigen receptor (CAR) that specifically targets cells expressing a specific antigen, for example, a tumor-associated antigen (TAA). CAR T-cell therapy has shown promising therapeutic effects in treating certain types of cancer. However, its application is often limited by toxicity, for example, undesired elevation of cytokine levels (known as cytokine release syndrome), which could lead to death of recipients Morgan et al., Molecular Therapy 18(4): 843-851, 2010. In addition, modified immune cells may not expand well in patients, may not persist long enough in vivo, and may be susceptible to the cytotoxic environment initiated by their own activities in vivo.

Antigen-presenting cells (APCs) are immune cells that mediate cellular immune responses by processing and presenting antigens for recognition by certain lymphocytes such as T cells. Classical APCs include dendritic cells, macrophages, Langerhans cells, and B cells.

### SUMMARY OF THE INVENTION

The present disclosure is based, at least in part, on the development of the use of antigen-presenting cells to booster therapeutic efficacy of CAR T cells. Without being bound by theory, co-use of the antigen-presenting cells disclosed herein is expected to enhance CAR-T cell in vivo expansion, leading to enhanced clinical efficacy.

Accordingly, the present disclosure features, in some aspects, a method for treating tumor, comprising administering to a subject in need thereof: (a) an effective amount of a population of genetically engineered T cells expressing one or more chimeric antigen receptors (CARs); and (b) an effective amount of antigen presenting cells (APCs). In some embodiments, the subject may be a human patient having a solid tumor. In other embodiments, the subject may be a human patient having a hematopoietic cancer, for example, acute myeloblastic leukemia (AML).

In some embodiments, the genetically engineered T cells may express a bi-specific CAR (e.g., containing a single fusion polypeptide or two fusion polypeptides) comprising a first antigen binding moiety specific to a tumor-associated antigen (TAA) and a second antigen binding moiety specific to CD19 or B-cell maturation antigen (BCMA). In some instances, the TAA is different from CD19 or BCMA. In other embodiments, the genetically engineered T cells may express a T cell receptor (TCR) specific to a TAA and a CAR, which comprises an antigen binding moiety specific to CD19 or BCMA. The APCs for use in the method disclosed herein may express CD19, BCMA, or a combination thereof. In some embodiments, the APCs may further express the TAA.

In some embodiments, the genetically engineered T cells for use in any of the methods disclosed herein may further express an antagonist of a cytokine, for example, a cytokine capable of activating immune responses. Examples include, but are not limited to, interleukin-1 (IL-1), interleukin-1 (IL-2), interleukin-6 (IL-6), interleukin-7 (IL-7), interleukin-1 (IL-9), interleukin-10 (IL-10), interleukin-12 (IL-12), interleukin-15 (IL-15), interleukin-18 (IL-18), interleukin-21 (IL-21), interleukin-23 (IL-23), interleukin-24 (IL-24), interleukin-33 (IL-33), interleukin-36 (IL-36), GM-CSF, interferon gamma (IFNγ), and Chemokine (C-C motif) ligand 19 (CCL19). In some examples, the antagonist of the cytokine can be a fusion polypeptide comprising a binding moiety (e.g., a receptor or the binding fragment thereof) to the cytokine and an immune activating cytokine, (e.g., IL-2, IL-7, IL-9, IL-10, IL-12, IL-15, IL-18, IL-21, IL-23, IL-24, IL-36, IL-33, and CCL19).

In one example, the fusion polypeptide comprises a binding moiety to IFNγ fused to IL-18, for example, those disclosed herein. Without being bound by theory, this fusion protein would be expected to enhance CAR-T cell efficacy via the IL-18 moiety while inhibiting cytokine toxicity mediated by IFNγ via the anti-IFNγ moiety in the fusion protein. In specific examples, the binding moiety to IFNγ is anti-IFNγ scFv, which preferably comprises the amino acid sequence of SEQ ID NO: 55. Alternatively or in addition, the IL-18 may comprise the amino acid sequence of SEQ ID NO: 53. In one example, the fusion polypeptide comprises the amino acid sequence of SEQ ID NO: 56.

In some embodiments, the genetically engineered T cells for use in any of the methods disclosed herein may express a bi-specific CAR. For example, the bi-specific CAR may comprise the first antigen binding moiety specific to the TAA and the second antigen binding moiety specific to CD19. Genetically engineered T cells expressing such a bi-specific CAR may be co-used with APCs expressing the CD19 and optionally the TAA. In other examples, the bi-specific CAR comprises the first antigen binding moiety specific to the TAA and the second antigen binding moiety specific to the BCMA. Genetically engineered T cells expressing such a bi-specific CAR may be in co-use with APCs expressing the BCMA and optionally the TAA.

In some instances, the bi-specific CAR disclosed herein may comprise a fusion polypeptide comprising the first antigen binding moiety and the second antigen binding moiety. In some examples, the first antigen binding moiety and the second antigen may be connected via a peptide linker. In some examples, the first antigen binding moiety and/or the second antigen binding moiety may be in a single-chain variable fragment (scFv) format. Alternatively, the first antigen binding moiety and/or the second antigen binding moiety may be in a single domain antibody (VHH) format. The bi-specific CAR may further comprise an intracellular signaling domain(s), which may comprise one or more signaling domains (e.g., a co-stimulatory domain and a cytoplasmic signaling domain); and optionally a hinge domain and a transmembrane domain connecting the antigen binding moieties and the intracellular signaling domain(s). In some specific examples, the bi-specific CAR can be in the format depicted in FIG. 2A.

In some instances, the bi-specific CAR may comprise a first fusion polypeptide that comprises the first antigen binding moiety and a second fusion polypeptide that comprises the second antigen binding moiety. In some examples, the first antigen binding moiety and/or the second antigen binding moiety may be or in a single-chain variable fragment (scFv) format. Alternatively, the first antigen binding moiety and/or the second antigen binding moiety may be in a single domain antibody (VHH) format. In yet another example, the first antigen binding moiety may be in a single-chain variable fragment (scFv) or in a single domain antibody (VHH) format. The second antigen binding moiety may be an extracellular domain of a ligand that binds the TAA. In some examples, the bi-specific CAR constructs may be in the format depicted in FIG. 3A. In some specific examples, the first antigen binding moiety is an extracellular domain of CD27, which binds CD70 and the second antigen binding moiety is specific to BCMA. Genetically engineered T cells expressing such a pair of bi-specific CARs may be used for treating AML, optionally in combination with APCs expressing BCMA, and optionally CD70.

In some examples, the first fusion polypeptide and the second fusion polypeptide comprise an intracellular signaling domain(s), which may comprise one or more signaling domains (e.g., a co-stimulatory signaling domain and a cytoplasmic signaling domain); and optionally a hinge domain and a transmembrane domain connecting the antigen binding moieties and the intracellular domain.

In some specific examples, the bi-specific CAR comprises one or more scFv fragments specific to CD19, BCMA, or a TAA as disclosed herein. Alternatively or in addition, the bi-specific CAR may comprise one or more VHH fragments specific to CD19, BCMA, or the TAA as disclosed herein. Exemplary scFv fragments and/or VHH fragments for use in constructing the bi-specific CAR constructs discloses herein are provided below. See also the Sequence Table, all of which are within the scope of the present disclosure.
(a) an scFv fragment specific to BCMA (anti-BCMA scFv), which comprises a heavy chain variable region (VH) and a light chain variable region (VL). In some instances, the VH of the anti-BCMA scFv comprises the amino acid sequence of SEQ ID NO: 14 and the VL of the anti-BCMA scFv comprises the amino acid sequence of SEQ ID NO: 15. In one specific example, the anti-BCMA scFv comprises the amino acid sequence of SEQ ID NO: 16.
(b) an scFv fragment specific to CD19 (anti-CD19 scFv), which comprises a heavy chain variable region (VH) and a light chain variable region (VL), wherein optionally the VH of the anti-CD19 scFv comprises the amino acid sequence of SEQ ID NO: 59 and the VL of the anti-CD19 scFv comprises the amino acid sequence of SEQ ID NO: 60, preferably wherein the anti-CD19 scFv comprises the amino acid sequence of SEQ ID NO: 61 or 62;
(c) an scFv fragment specific to Meso (anti-Meso scFv), which comprises a heavy chain variable region (VH) and a light chain variable region (VL). In some instances, the VH of the anti-Meso scFv comprises the amino acid sequence of SEQ ID NO: 11 and the VL of the anti-Meso scFv comprises the amino acid sequence of SEQ ID NO: 12. In one example, the anti-Meso scFv comprises the amino acid sequence of SEQ ID NO: 13.
(d) an scFv fragment specific to HER2 (anti-HER2 scFv), which comprises a heavy chain variable region (VH) and a light chain variable region (VL). In some instances, the VH of the anti-HER2 scFv comprises the amino acid sequence of SEQ ID NO: 17 and the VL of the anti-HER2 scFv comprises the amino acid sequence of SEQ ID NO: 18. In one example, the anti-HER2 scFv comprises the amino acid sequence of SEQ ID NO: 19.
(e) an scFv fragment specific to GPC3 (anti-GPC3 scFv), which comprises a heavy chain variable region (VH) and a light chain variable region (VL). In some instances, the VH of the anti-GPC3 scFv comprises the amino acid sequence of SEQ ID NO: 20 and the VL of the anti-GPC3 scFv comprises the amino acid sequence of SEQ ID NO: 21. In one example, the anti-BCMA scFv comprises the amino acid sequence of SEQ ID NO: 22.
(f) an scFv fragment specific to Claudin 18.2 (anti-Claudin 18.2 scFv), which comprises a heavy chain variable region (VH) and a light chain variable region (VL). In some instances, the VH of the anti-Claudin 18.2 scFv comprises the amino acid sequence of SEQ ID NO: 23 and the VL of the anti-Claudin 18.2 scFv comprises the amino acid sequence of SEQ ID NO: 24. In one example, the anti-Claudin 18.2 scFv comprises the amino acid sequence of SEQ ID NO: 25.
(g) anti-CD33VHH comprising the amino acid sequence of SEQ ID NO:42; and
(h) anti-CD123 VHH comprising the amino acid sequence of any one of SEQ ID NOS 47-52;
(i) anti-HER2 VHH comprising the amino acid sequence of SEQ ID NO: 69 or 72;
(j) anti-Claudin 18.2 VHH comprising the amino acid sequence of SEQ ID NO: 75 or 78;
(k) anti-mesothelin VHH comprising the amino acid sequence of SEQ ID NO: 81 or 84;
(l) anti-PSMA VHH comprising the amino acid sequence of SEQ ID NO: 87 or 90;
(m) anti-GPC3 VHH comprising the amino acid sequence of SEQ ID NO: 93; or
(n) anti-EGFR VHH comprising the amino acid sequence of SEQ ID NO: 96.

In some instances, the bi-specific CAR for use in the present disclosure binds Meso and BCMA. The anti-Meso/BCMA bi-specific CAR may comprise a fusion polypeptide. In one example, the anti-Meso/BCMA bi-specific CAR comprises the amino acid sequence of 26 (with signal peptide) or 27 (without signal peptide). In another example, the anti-Meso/BCMA bi-specific CAR comprises the amino acid sequence of 107 (with signal peptide) or 108 (without signal peptide). In yet another example, the anti-Meso/BCMA bi-specific CAR comprises the amino acid sequence of 109 (with signal peptide) or 110 (without signal peptide). Alternatively, the anti-Meso/BCMA bi-specific CAR may comprise two separately CAR polypeptides, one binding to Mesothelin and the other binding to BCMA. In one example, the anti-meso CAR polypeptide comprises the amino acid sequence of SEQ ID NO: 82 (with signal peptide) or 83 (without signal peptide). In another example, the anti-meso CAR polypeptide comprises the amino acid sequence of SEQ ID NO: 85 (with signal peptide) or 86 (without signal peptide). The anti-BCMA CAR polypeptide may comprise the amino acid sequence of SEQ ID NO: 38 (with signal peptide) or 39 (without signal peptide). Alternatively, the anti-BCMA CAR polypeptide may comprise the amino acid sequence of SEQ ID NO: 125 (with signal peptide) or 126 (without signal peptide).

In some instances, the bi-specific CAR for use in the present disclosure binds HER2 and BCMA. The anti-HER2/BCMA bi-specific CAR may comprise a fusion polypeptide. In one example, the anti-HER2/BCMA bi-specific CAR comprises the amino acid sequence of 28 (with signal peptide) or 29 (without signal peptide). In another example, the anti-HER2/BCMA bi-specific CAR comprises the amino acid sequence of 99 (with signal peptide) or 100 (without signal peptide). In yet another example, the anti-HER2/BCMA bi-specific CAR comprises the amino acid sequence of 101 (with signal peptide) or 102 (without signal peptide). Alternatively, the anti-HER2/BCMA bi-specific CAR may comprise two separately CAR polypeptides, one binding to HER2 and the other binding to BCMA. In one example, the anti-HER2 CAR polypeptide comprises the amino acid sequence of SEQ ID NO: 70 (with signal peptide) or 71 (without signal peptide). In another example, the anti-HER2 CAR polypeptide comprises the amino acid sequence of SEQ ID NO: 73 (with signal peptide) or 74 (without signal peptide). The anti-BCMA CAR polypeptide may comprise the amino acid sequence of SEQ ID NO: 38 (with signal peptide) or 39 (without signal peptide). Alternatively, the anti-BCMA CAR polypeptide may comprise the amino acid sequence of SEQ ID NO: 125 (with signal peptide) or 126 (without signal peptide).

In some instances, the bi-specific CAR for use in the present disclosure binds GPC3 and BCMA. The anti-GPC3/BCMA bi-specific CAR may comprise a fusion polypeptide. In one example, the anti-GPC3/BCMA bi-specific CAR comprises the amino acid sequence of 30 (with signal peptide) or 31 (without signal peptide). In another example, the anti-GPC3/BCMA bi-specific CAR comprises the amino acid sequence of 115 (with signal peptide) or 116 (without signal peptide). Alternatively, the anti-GPC3/BCMA bi-specific CAR may comprise two separately CAR polypeptides, one binding to GPC3 and the other binding to BCMA. In one example, the anti-GPC3CAR polypeptide comprises the amino acid sequence of SEQ ID NO: 94 (with signal peptide) or 95 (without signal peptide). The anti-BCMA CAR polypeptide may comprise the amino acid sequence of SEQ ID NO: 38 (with signal peptide) or 39 (without signal peptide). Alternatively, the anti-BCMA CAR polypeptide may comprise the amino acid sequence of SEQ ID NO: 125 (with signal peptide) or 126 (without signal peptide).

In some instances, the bi-specific CAR for use in the present disclosure binds Claudin 18.2 and BCMA. The anti-Claudin 18.2/BCMA bi-specific CAR may comprise a fusion polypeptide. In one example, the anti-Claudin 18.2/BCMA bi-specific CAR comprises the amino acid sequence of 32 (with signal peptide) or 33 (without signal peptide). In another example, the anti-Claudin 18.2/BCMA bi-specific CAR comprises the amino acid sequence of 103 (with signal peptide) or 104 (without signal peptide). In yet another example, the anti-Claudin 18.2/BCMA bi-specific CAR comprises the amino acid sequence of 105 (with signal peptide) or 106 (without signal peptide). Alternatively, the anti-Claudin 18.2/BCMA bi-specific CAR may comprise two separately CAR polypeptides, one binding to Claudin 18.2 and the other binding to BCMA. In one example, the anti-Claudin 18.2 CAR polypeptide comprises the amino acid sequence of SEQ ID NO: 76 (with signal peptide) or 77 (without signal peptide). In another example, the anti-Claudin 18.2 CAR polypeptide comprises the amino acid sequence of SEQ ID NO: 79 (with signal peptide) or 80 (without signal peptide). The anti-BCMA CAR polypeptide may comprise the amino acid sequence of SEQ ID NO: 38 (with signal peptide) or 39 (without signal peptide). Alternatively, the anti-BCMA CAR polypeptide may comprise the amino acid sequence of SEQ ID NO: 125 (with signal peptide) or 126 (without signal peptide).

In some instances, the bi-specific CAR for use in the present disclosure binds CD19 and BCMA. The anti-CD19/BCMA bi-specific CAR may comprise a fusion polypeptide. In one example, the anti-CD19/BCMA bi-specific CAR comprises the amino acid sequence of 57 (with signal peptide) or 58 (without signal peptide). Alternatively, the anti-CD19/BCMA bi-specific CAR may comprise two separately CAR polypeptides, one binding to CD19 and the other binding to BCMA.

In some instances, the bi-specific CAR for use in the present disclosure binds CD70 and BCMA. The anti-CD70/BCMA bi-specific CAR may comprise a fusion polypeptide. Alternatively, the anti-CD70/BCMA bi-specific CAR may comprise two separately CAR polypeptides, one binding to CD70 and the other binding to BCMA. In one example, the anti-CD70 CAR polypeptide comprises the amino acid sequence of SEQ ID NO: 36 (with signal peptide) or 37 (without signal peptide). The anti-BCMA CAR polypeptide may comprise the amino acid sequence of SEQ ID NO: 38 (with signal peptide) or 39 (without signal peptide). Alternatively, the anti-BCMA CAR polypeptide may comprise the amino acid sequence of SEQ ID NO: 125 (with signal peptide) or 126 (without signal peptide).

In some instances, the bi-specific CAR for use in the present disclosure binds PSMA and BCMA. The anti-PSMA/BCMA bi-specific CAR may comprise a fusion polypeptide. In one example, the anti-PSMA/BCMA bi-specific CAR comprises the amino acid sequence of 111 (with signal peptide) or 112 (without signal peptide). In another example, the anti-PSMA/BCMA bi-specific CAR comprises the amino acid sequence of 113 (with signal peptide) or 114 (without signal peptide). Alternatively, the anti-PSMA/BCMA bi-specific CAR may comprise two separately CAR polypeptides, one binding to PSMA and the other binding to BCMA. In one example, the anti-PSMA CAR polypeptide comprises the amino acid sequence of SEQ ID NO: 88 (with signal peptide) or 89 (without signal peptide). In another example, the anti-PSMA CAR polypeptide comprises the amino acid sequence of SEQ ID NO: 91 (with signal peptide) or 92 (without signal peptide). The anti-BCMA CAR polypeptide may comprise the amino acid sequence of SEQ ID NO: 38 (with signal peptide) or 39 (without signal peptide). Alternatively, the anti-BCMA CAR polypeptide may comprise the amino acid sequence of SEQ ID NO: 125 (with signal peptide) or 126 (without signal peptide).

In some instances, the bi-specific CAR for use in the present disclosure binds EGFR and BCMA. The anti-EGFR/BCMA bi-specific CAR may comprise a fusion polypeptide. In one example, the anti-EGFR/BCMA bi-specific CAR comprises the amino acid sequence of 117 (with signal peptide) or 118 (without signal peptide). Alternatively, the anti-EGFR/BCMA bi-specific CAR may comprise two separately CAR polypeptides, one binding to EGFR and the other binding to BCMA. In one example, the anti-EGFR CAR polypeptide comprises the amino acid sequence of SEQ ID NO: 97 (with signal peptide) or 98 (without signal peptide). The anti-BCMA CAR polypeptide may comprise the amino acid sequence of SEQ ID NO: 38 (with signal peptide) or 39 (without signal peptide). Alternatively, the anti-BCMA CAR polypeptide may comprise the amino acid sequence of SEQ ID NO: 125 (with signal peptide) or 126 (without signal peptide).

In other specific examples, the bi-specific CAR comprises a first antigen binding moiety that is an extracellular domain of CD27, which binds CD70 and a second antigen binding moiety that is specific to BCMA. In some instances, the anti-BCMA moiety may comprise an scFv fragment specific to BCMA (anti-BCMA scFv), which comprises a heavy chain variable region (VH) and a light chain variable region (VL). In some examples, the VH of the anti-BCMA scFv comprises the amino acid sequence of SEQ ID NO: 14 and the VL of the anti-BCMA scFv comprises the amino acid sequence of SEQ ID NO: 15. In one example, the anti-BCMA scFv comprises the amino acid sequence of SEQ ID NO: 16. In one specific example, the anti-BCMA CAR comprises the amino acid sequence of SEQ ID NO: 38 or 39. In another specific example, the anti-BCMA CAR polypeptide may comprise the amino acid sequence of SEQ ID NO: 125 (with signal peptide) or 126 (without signal peptide). Alternatively or in addition, the extracellular domain of CD27 may comprise the amino acid sequence of SEQ ID NO: 34 or 35.

In yet other instances, the population of genetically engineered T cells may express a T cell receptor (TCR) specific to the TAA and a CAR comprising an antigen binding moiety specific to CD19 or BCMA. In some examples, the CAR comprises the antigen binding moiety specific to CD19. Genetically engineered T cells expressing the TCR and the CAR may be in co-use with APCs expressing CD19 and optionally the TAA. In other examples, the CAR may comprise the antigen binding moiety specific to BCMA. Genetically engineered T cells expressing the TCR and the CAR may be co-used with APCs expressing BCMA and optionally the TAA. In some examples, the antigen binding moiety is in a single-chain variable fragment (scFv) format or in a single domain antibody (VHH) format. In some specific examples, the TCR specific to the TAA and the CAR may be in the format depicted in FIG. 4A. In one example, the T cell receptor (TCR) may be specific to NY-ESO-1, which optionally comprises a TCRα chain comprising the amino acid sequence of SEQ ID NO: 40 and a TCRβ chain comprising the amino acid sequence of SEQ ID NO: 41.

In some examples, the TCR disclosed herein may be a complex comprising a first fusion polypeptide that comprises an antigen binding moiety to CD33, and a second fusion polypeptide that comprises an antigen binding moiety to CD123. In some instances, the first fusion polypeptide may further comprise a transmembrane fragment of CD3δ and the second fusion polypeptide may further comprise a transmembrane fragment of CD3γ. Alternatively, the first fusion polypeptide may further comprise a transmembrane fragment of CD3γ and the second fusion polypeptide may further comprise a transmembrane fragment of CD3δ. In some examples, the transmembrane fragments of CD3δ and CD3γ are free of intracellular domains of the CD3δ and CD3γ. In some specific examples, the TCR-based bi-specific CAR and the co-expressed CAR may be in the format depicted in FIG. 5A.

In some examples, such a T cell receptor (TCR) comprises a modified CD3δ chain and a modified CD3γ chain, which collectively comprises a first antigen binding moiety specific to CD33 (anti-CD33 moiety) and a second antigen binding moiety specific to CD123 (anti-CD123 moiety). In some instances, the modified CD3δ chain comprises an extracellular and transmembrane domain of CD3δ fused to the anti-CD33 moiety, the modified CD3γ chain comprises an extracellular and transmembrane domain of CD3γ fused to the anti-CD123 moiety, or vice versa. In specific examples:
(a) the modified CD3δ chain comprises the amino acid sequence of SEQ ID NO: 45;
(b) the modified CD3γ chain comprises the amino acid sequence of SEQ ID NO: 46;
(c) the anti-CD33 moiety is an anti-CD33VHH, which optionally comprises the amino acid sequence of SEQ ID NO: 42; and/or
(d) the anti-CD123 moiety is an anti-CD123 VHH, which optionally comprises the amino acid sequence of any one of SEQ ID NOs: 47-52.

Alternatively or in addition, the antigen binding moiety specific to CD19 in the CAR comprises a VH comprising SEQ ID NO: 59 and a VL comprising SEQ ID NO: 60. In some examples, the antigen binding moiety specific to CD19 in the CAR is an scFv fragment comprising the amino acid sequence of SEQ ID NO: 61 or 62.

Alternatively or in addition, the antigen binding moiety specific to BCMA in the CAR comprises a VH comprising SEQ ID NO: 14 and a VL comprising SEQ ID NO: 15. In some examples, the antigen binding moiety specific to BCMA in the CAR is an scFv fragment comprising the amino acid sequence of SEQ ID NO: 16. In specific examples, the CAR is an anti-BCMA CAR comprising the amino acid sequence of SEQ ID NO: 38 or 39. In other specific examples, the anti-BCMA CAR polypeptide may comprise the amino acid sequence of SEQ ID NO: 125 (with signal peptide) or 126 (without signal peptide).

Any of the CAR disclosed herein may further comprise an intracellular signaling domain(s), which may comprise one or more signaling domains (e.g., a co-stimulatory signaling domain and a cytoplasmic signaling domain); and optionally a hinge domain and a transmembrane domain connecting the antigen binding moieties and the intracellular domain. In some examples, the CAR comprises the hinge domain, which may be of CD8, CD28, CD4, CD3, or an IgG molecule. Alternatively or in addition, the CAR may comprise a transmembrane domain, which may be from CD3, CD4, CD8, CD27 or CD28. In some examples, the CAR comprises intracellular signaling domains that comprise a co-stimulatory signaling domain and a cytoplasmic signaling domain. Exemplary signaling domains for use as components of the intracellular signaling domains in any of the CARs disclosed herein include those from CD3, FcR, DAP12, 41BB, OX40, CD28, CD27, ICOS, IL-2R, IL-7R, IL-9R, IL-10R, IL-12R, IL18R, IL-21R, or IL-23R, or a combination thereof. In one example, the intracellular signaling domains comprise a co-stimulatory domain of 4-1BB, an IL2Rb signaling domain, and a CD3ζ signaling domain. In some specific examples, the co-stimulatory domain of 4-1BB comprises the amino acid sequence of SEQ ID NO: 8, the IL2Rb signaling domain comprises the amino acid sequence of SEQ ID NO:9, and/or the CD3ζ signaling domain comprises the amino acid sequence of SEQ ID NO: 10.

In some embodiments, the population of genetically engineered T cells comprise tumor infiltrating T cells (TILs). In some embodiments, the population of genetically engineered T cells are autologous to the subject. Alternatively, the population of genetically engineered T cells are allogeneic to the subject.

In some embodiments, the APC cells comprise immune cells, stem cells or tumor cells. In some examples, the immune cells can be, but are not limited to, T-cells, Natural Killer (NK) cells, tumor infiltrating lymphocytes, dendritic cells, macrophages, B cells, neutrophils, eosinophils, basophils, mast cells, myeloid-derived suppressor cells, and/or mesenchymal stem cells. In some examples, the stem cells can be mesenchymal stem cells. In some examples, the tumor cells can be K562 cells. In some instances, the APCs are native cells expressing CD19 and/or BCMA, and optionally the TAA. Alternatively, the APCs are genetically engineered to express the CD19 and/or the BCMA, and optionally the TAA. In some instances, the APCs are derived from peripheral blood cells, cord blood cells, induced pluripotent stem cells (iPSCs), or an immune cell line. In some examples, the APCs are autologous to the subject. Alternatively, the APCs are allogeneic to the subject.

Any of the APCs disclosed herein may be genetically engineered to further express a membrane bound stimulatory cytokine. Examples include IL-10, IL-18, IL-15, IL-9, or IL-21. See examples provided in the Sequence Table below.

Further, the present disclosure provides a kit for treating cancer, comprising: (a) any of the population of genetically engineered T cells disclosed herein, and (b) any of the APCs disclosed herein. Also within the scope of the present disclosure are such a kit for use in cancer treatment, as well as uses of the kit for manufacturing a medicament for use in cancer therapy.

Any of the genetically engineered T cells expressing the bi-specific CAR or the TCR and CAR is within the scope of the present disclosure. Any of the genetically engineered APC cells as disclosed herein is also within the scope of the present disclosure.

The details of one or more embodiments of the invention are set forth in the description below. Other features or advantages of the present invention will be apparent from the following drawings and detailed description of several embodiments, and from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is a schematic illustration depicting antigen-presenting cells expressing CD19 and/or BCMA.

FIGS. 2A-2C include diagrams showing anti-tumor activity of genetically engineered T cells expressing a bi-specific chimeric antigen receptor (CAR) targeting BCMA and a tumor specific antigen (TAA). FIG. 2A: a schematic illustration depicting a bi-specific CAR in a single fusion polypeptide format, in which two antigen binding moieties are in tandem repeat. One antigen binding moiety may be specific to a tumor associated antigen (TAA), for example, mesothelin, GPC3, Claudin 18.2, HER2, PSMA, or EGFR. The other antigen binding moiety may be specific to CD19 or BCMA. The CAR polypeptide may further comprise a hinge and transmembrane domain (e.g., from CD8) and intracellular signaling domains (e.g., 4-1BB co-stimulatory domain, truncated IL2Rb signaling domain, and CD3ζ signaling domain). FIG. 2B: a diagram showing lysis efficiency of BCMA+MM1 Scells by genetically engineered T cells expressing the tandem bi-specific CAR targeting BCMA and one of the TAAs as indicated. FIG. 2C: a diagram showing lysis efficiency of TAA expressing tumor cells by genetically engineered T cells expressing the tandem bi-specific CAR targeting BCMA and one of the TAAs as indicated.

FIGS. 3A-3B include diagrams showing anti-tumor activity of genetically engineered T cells expressing two CAR constructs. FIG. 3A: a schematic illustration depicting a bi-specific CAR in a two-chain format, each comprising one antigen binding moieties. One antigen binding moiety can be an extracellular domain of a ligand of a tumor associated antigen, for example, an extracellular domain of CD27, which is a ligand of CD70. The extracellular domain of the ligand may be fused to an intracellular signaling domain, for example, the CD3ζ signaling domain. The other antigen binding moiety may be specific to CD19 or BCMA. The CAR polypeptide comprising such may further comprise a hinge and transmembrane domain (e.g., from CD8) and intracellular signaling domains (e.g., 4-1BB co-stimulatory domain, truncated IL2Rb signaling domain, and CD3ζ signaling domain). FIG. 3B: a diagram showing tumor cell lysis efficiency of genetically engineered T cells expressing one CAR targeting CD70 and one CAR targeting BCMA against various tumor cells as indicated.

FIGS. 4A-4B include diagrams showing anti-tumor activity of genetically engineered T cells expressing TCR specific to TAA and a CAR targeting BCMA or CD19. FIG. 4A: a schematic illustration depicting genetically engineered T cells expressing a TCR specific to a TAA such as NY-ESO-1 and a CAR comprising an antigen binding moiety to CD19 or BCMA. The CAR may further comprise a hinge and transmembrane domain (e.g., from CD8) and intracellular signaling domains (e.g., 4-1BB co-stimulatory domain, truncated IL2Rb signaling domain, and CD3ζ signaling domain). FIG. 4B: a diagram showing tumor cell lysis efficiency of genetically engineered T cells expressing the NY-ESO-1-specific TCR and the CAR targeting BCMA against various tumor cells as indicated.

FIGS. 5A-5C include diagrams showing anti-tumor activity of genetically engineered T cells expressing TCR-based bi-specific CAR and a separate CAR construct targeting BCMA or CD19. FIG. 5A: a schematic illustration depicting genetically engineered T cells expressing a modified TCR specific to TAAs and a CAR comprising an antigen binding moiety to CD19 or BCMA. The modified TCR comprises a first chain that comprises an antigen binding moiety to a first TAA (e.g., a VHH targeting CD3ζ) and a second chain that comprises an antigen binding moiety to a second TAA (e.g., a VHH targeting CD123). Each VHH can be fused to a fragment of CD3δ or CD3γ, which does not contain the intracellular domain. The CAR may further comprise a hinge and transmembrane domain (e.g., from CD8) and intracellular signaling domains (e.g., 4-1BB co-stimulatory domain, truncated IL2Rb signaling domain, and CD3 signaling domain). FIG. 5B: a diagram showing cytotoxicity of genetically engineered T cells expressing TCR-based bi-specific CARs targeting CD3ζ and CD123 and anti-BCMA CAR against RPMI8226 B lymphocytes. FIG. 5C: a diagram showing cytotoxicity of genetically engineered T cells expressing TCR-based bi-specific CARs targeting CD3ζ and CD123 and anti-BCMA CAR against Molm13 human leukemia cells. Numbers 1-6 at x-axis correspond to anti-CD123 VHH1-anti-CD123 VHH6 listed in the Sequence Table.

FIGS. 6A-6C include diagrams showing that co-culture with antigen-presenting cells (APCs) enhanced CAR-T cell expansion. FIG. 6A: genetically engineered T cells expressing bi-specific CAR targeting CD70 and BCMA co-cultured with K562 cells, NK92 cells expressing GFP/BCMA, or NK92 cells expressing CD19/BCMA. FIG. 6B: genetically engineered T cells expressing bispecific anti-NY-ESO-1 TCR/anti-BCMA CAR co-cultured with K562 cells, NK92 cells expressing GFP/BCMA, or NK92 cells expressing CD19/BCMA. FIG. 6C: genetically engineered T cells expressing bispecific CAR targeting CD19 and BCMA co-cultured with K562 cells expressing GFP/BCMA, or NK92 cells expressing CD19/BCMA.

FIGS. 7A-7D include diagrams showing in vivo expansion cytotoxicity against target cells of CAR-T cell co-administered with APCs. FIG. 7A: a diagram showing in vitro expansion of CAR-T cells co-cultured with K562, MM1S, or CD19/BCMA-expressing NK92 cells as indicated. FIG. 7B: a diagram showing in vitro cytotoxicity of CAR-T cells against the various target cells as indicated. FIG. 7C: a diagram showing in vivo expansion of CAR-T cells co-administered with antigen-presenting cells. FIG. 7D: a diagram showing the levels of CD123+, CD33+, and CD123+CD33+ cells in the human MM patient treated with both the CAR-T cells and the APCs.

### DETAILED DESCRIPTION OF THE INVENTION

The clinical success of CAR-T therapy in treating hematological cancers relies on robust CAR-T cell expansion in patients, which can be driven by the interaction between the CAR expressed on the CAR-T cells and the antigen presented on tumor cells, to which the CAR binds. However, initial clinical trials of solid tumors with CAR-T therapy showed very limited expansion of CAR-T cells in patients. It is suggested that this observation may be due to the limited presence of the target solid tumor antigen in local tumor regions and not available in systemic blood circulation.

To solve this problem, the present disclosure aims at using antigen-presenting cells (APCs) that display one or more target antigens concurrently with CAR-T cells targeting at least one of such antigens. With being bound by theory, such APCs would be expected to present in blood circulation, which would enhance CAR-T cell proliferation and expansion, leading to enhanced infiltration into diseased tissues such as solid tumor tissues and thus higher therapeutic efficacy against the target disease (e.g., solid tumors).

Accordingly, described herein are compositions comprising genetically engineered T cells expressing chimeric antigen receptors (CARs) or TCRs targeting specific antigens (e.g., tumor-associated antigens or TAAs) and antigen-presenting cells expressing CD19 and/or BCMA, and optionally the TAA for use in treating cancer, including solid tumors and hematopoietic cancers such as AML. As reported herein, antigen-presenting cells successfully enhanced CAR-T cell expansion both in vitro and in vivo. The combined cancer therapies provided herein therefore are expected to be more effective relative to the treatments that comprise only the CAR-T cells.

### I. Genetically Engineered T Cells

In some aspects, provided herein are genetically engineered T cells either expressing a bi-specific CAR specific to CD19 or BCMA and a tumor associated antigen (TAA), or expressing a T cell receptor (e.g., modified, a.k.a., TCR-based chimeric antigen receptor) specific to a TAA and a CAR specific to CD19 or BCMA. Such genetically engineered T cells can be co-used with antigen presenting cells (APCs) expressing CD19 and/or BCMA, optionally the TAA as well, to enhance treatment efficacy.

A CAR disclosed herein is an artificial (non-naturally occurring) receptor having a binding specificity to a target antigen of interest (e.g., a tumor cell antigen) and capable of triggering immune responses in immune cells expression such upon binding to the target antigen. A CAR often comprises an extracellular antigen-binding domain fused to at least an intracellular signaling domain. Cartellieri et al., J Biomed Biotechnol 2010:956304, 2010. The TCR-based chimeric receptors as disclosed herein may be artificial TCRs or engineered TCRs having grafted artificial antigen specificity to target antigens.

### (A) Genetically Engineered T Cells Expressing Bi-Specific CARs

In some embodiments, provided herein are genetically engineered T cells expressing a bi-specific CAR. A bi-specific CAR as disclosed herein, which may be a single-chain fusion polypeptide, or includes multiple chains (e.g., two chains), is a CAR or CAR complex capable of binding to two different antigens or antigenic epitopes.

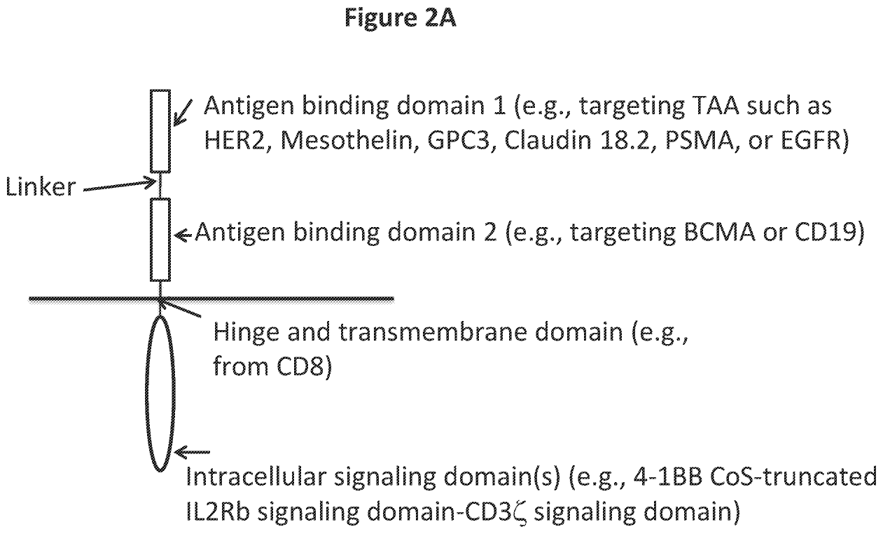

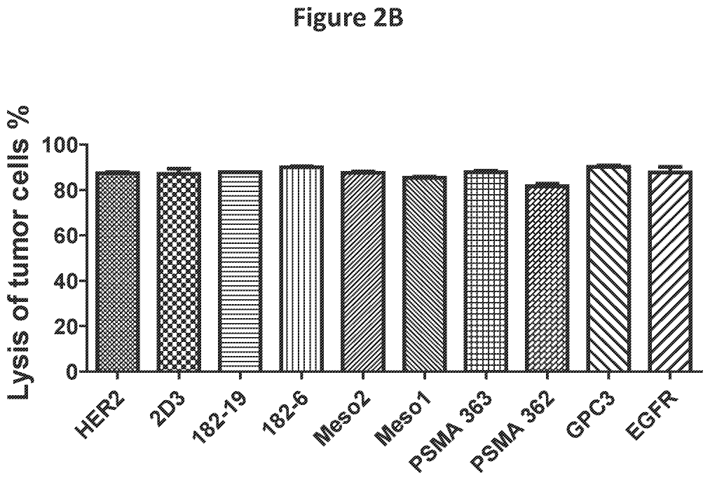

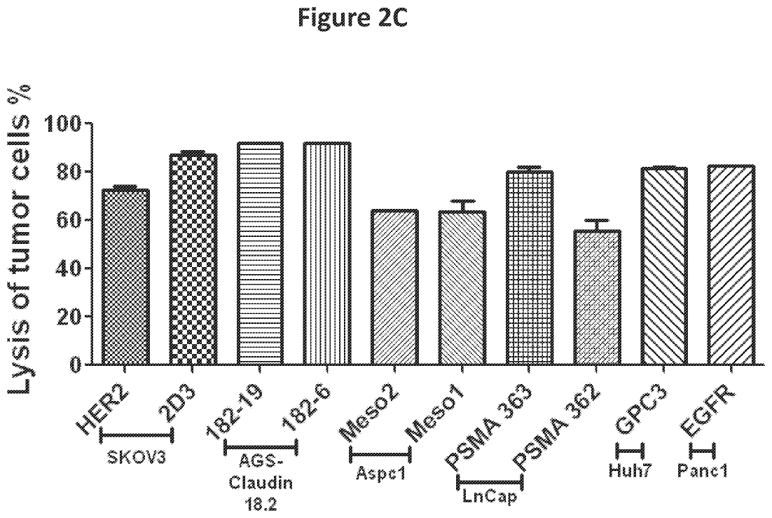

In some examples, the bi-specific CAR comprises a single polypeptide, which may comprise (a) an extracellular antigen binding domain comprising a first antigen-binding moiety, and a second antigen-binding moiety, (b) a hinge/transmembrane domain, and (c) one or more intracellular signaling domains (e.g., comprising a co-stimulatory signaling domain, and one or more cytoplasmic signaling domains). The two antigen-binding moieties may be in tandem repeat arrangement. See, e.g., FIG. 2A.

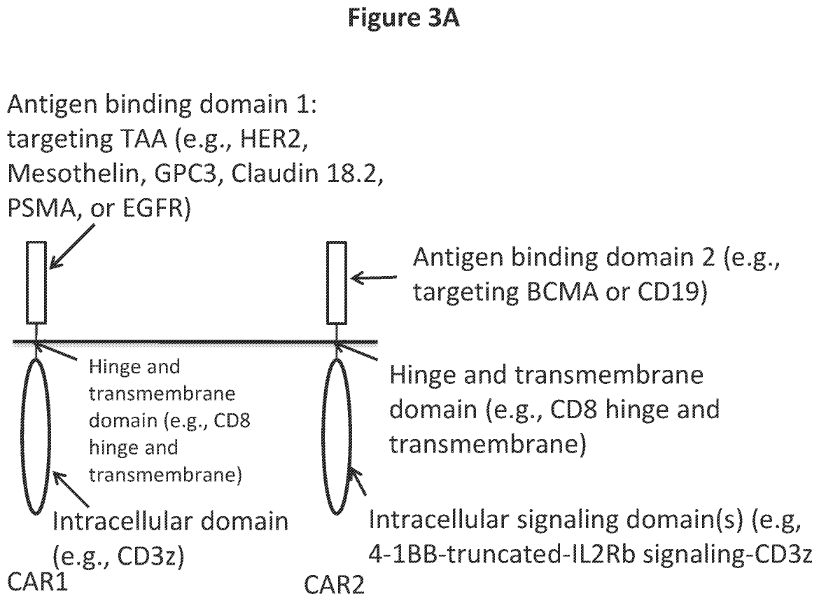

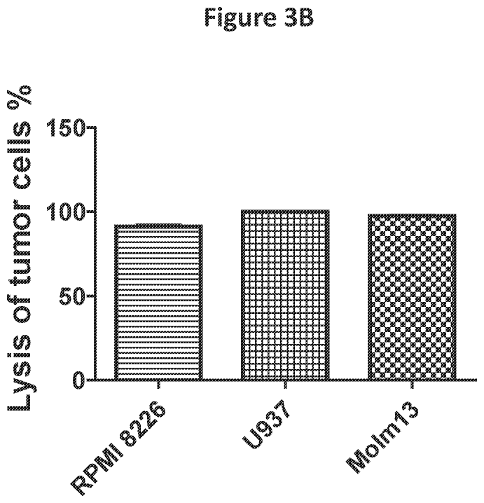

In other examples, the bi-specific CAR disclosed herein may comprise two separate CAR polypeptides. The first polypeptide (CAR 1) comprises an extracellular domain that includes a first antigen-binding moiety and intracellular signaling domains (e.g., comprising a co-stimulatory signaling domain, and one or more cytoplasmic signaling domains). The second polypeptide (CAR 2) comprises an extracellular domain that includes a second antigen-binding moiety and intracellular signaling domains (e.g., comprising a co-stimulatory signaling domain, and one or more cytoplasmic signaling domains). Either the first polypeptide or the second polypeptide, or both, may further comprise a hinge and transmembrane domain. In some examples, one of the two CAR polypeptides (e.g., CAR 1) is free of the hinge and transmembrane domain and the other polypeptide (e.g., CAR 2) includes such. See, e.g., FIG. 3A.

### (a) Extracellular Domains

The extracellular domains in the bi-specific CARs disclosed herein includes one or both antigen-binding moieties as disclosed herein.

In some instances, the first antigen-binding moiety and/or the second antigen-binding moiety may be in single chain variable fragment (scFv) format, in which an antibody heavy chain variable region (VH) and an antibody light chain variable region (VL) are connected, optionally via a flexible peptide linker. The scFv binding moiety may have the VH-VL orientation (from N-terminus to C-terminus). Alternatively, the scFv binding moiety may have the VL-VH orientation.

In other instances, the first antigen-binding moiety and/or the second antigen-binding moiety may be a single domain antibody fragment (e.g., a heavy chain only antibody or VHH). In some instances, one of the first antigen-binding moiety can be an scFv fragment and the other antigen-binding domain may be a VHH.

In the bi-specific CAR, the first antigen-binding moiety binds to CD19 or BCMA and the second antigen-binding moiety binds a tumor-associated antigen (TAA).

CD19 is a B-lymphocyte antigen expressed in B lineage cells. It is reported that CD19 acts as an adaptor protein to recruit cytoplasmic signaling protein to the membrane. It also works within the CD19/CD21 complex to decrease the threshold for B cell receptor signaling. CD19 is a well-characterized receptor in the art. As an example, the amino acid sequence of human CD19 is provided in the Sequence Table below.

The anti-CD19 antigen binding moiety in any of the bi-specific antibodies disclosed herein may be derived from an anti-CD19 antibody or an anti-CD19 CAR as known in the art. For example, the anti-CD19 antigen binding moiety may be derived from anti-CD19 antibody FMC-63. Alternatively, the anti-CD19 antigen binding moiety may be derived from tisagenlecleucel or brexucabtageneautoleucel. The anti-CD19 antigen binding moiety in the bi-specific antibodies disclosed herein may have the same heavy chain and light chain complementary determining regions (CDRs) or the same VH and VL fragments as those of the anti-CD19 antibody or anti-CD19 CAR known in the art. Alternatively, it may contain one or more variations as disclosed herein. In one example, the anti-CD19 moiety for use in making the anti-CD19 CAR may be derived from FMC63 (e.g., having the same heavy chain and light chain CDRs or having the same heavy chain variable region and light chain variable region as FMC63).

BCMA (B-cell maturation antigen) is a cell surface receptor that recognizes B-cell activating factor (BAFF). This receptor is preferentially expressed in mature B lymphocytes. It is reported that BCMA may be important for B cell development and autoimmune response. BCMA is used as a drug target for treatment of multiple myeloma. This receptor is well-characterized in the art. As an example, the amino acid sequence of human BCMA is provided in the Sequence Table below.

The anti-BCMA antigen binding moiety in any of the bi-specific antibodies disclosed herein may be derived from an anti-BCMA antibody or an anti-BCMA CAR as known in the art. For example, the anti-BCMA antigen binding moiety may be derived from the anti-BCMA antibody provided in the Sequence Table below. The anti-BCMA antigen binding moiety in the bi-specific antibodies disclosed herein may have the same heavy chain and light chain complementary determining regions (CDRs) or the same VH and VL fragments as provided in the Sequence Table. Alternatively, it may contain one or more variations as disclosed herein. In some specific examples, the anti-BCMA antigen binding moiety may be an scFv fragment, for example, the anti-BCMA scFv provided in the Sequence Table.

Tumor-associated antigen (TAA) refers to an antigen produced by tumor cells. In some examples, the TAA is a tumor specific antigen—an antigen expressed only by tumor cells or expressed by tumor cells at a much higher level than the expression level by non-cancerous cells. In some instances, the TAA disclosed here is not CD19 or BCMA. Non-limiting examples of TAAs include 5T4, CD2, CD3, CD5, CD7, CD20, CD22, CD30, CD33, CD38, CD70, CD123, CD133, CD171, CEA, CS1, BAFF-R, PSMA, PSCA, desmoglein (Dsg3), HER-2, FAP, FSHR, NKG2D, GD2, EGFRVIII, mesothelin, ROR1, MAGE, MUC1, MUC16, GPC3, Lewis Y, HER2, Claudin 18.2, and VEGFRII.

In some examples, the TAA may be mesothelin, HER2, Claudin 18.2, GPC3, or EGFR. Exemplary antibodies binding to these TAAs are provided in the Sequence Table. The anti-TAA antigen binding moiety in the bi-specific antibodies disclosed herein may have the same heavy chain and light chain complementary determining regions (CDRs) or the same VH and VL fragments as those provided in the Sequence Table. Alternatively, it may contain one or more variations as disclosed herein. In some specific examples, the anti-TAA antigen binding moiety may be an scFv fragment, for example, the anti-meso scFv, the anti-HER2 scFv, the anti-GPC3 scFv, or the anti-Claudin 18.2 scFv provided in the Sequence Table. In other specific examples, the anti-TAA antigen binding moiety may be a VHH fragment, for example, the anti-HER2 VHH, the anti-meso VHH, the anti-Claudin 18.2 VHH, the anti-PSMAVHH, the anti-GPC3 VHH, or the anti-EGFR VHH provided in the Sequence Table.

In some examples, the TAA may be CD33 or CD123. The antigen-binding moiety specific to CD33 or CD123 may be a VHH fragment, for example, those provided in the Sequence Table.

In other examples, TAA is a cell surface receptor, for example, an immune cell receptor. In that case, the antigen-binding moiety specific to the TAA may be an extracellular domain of a ligand specific to the receptor/TAA. For example, when the TAA is CD70, the antigen-binding moiety may be an extracellular domain of CD27, which is the ligand of CD70.

### (b) Intracellular Signaling Domains

The single-chain bi-specific CAR or the CAR 1/CAR 2 of the two-chain bi-specific CAR disclosed herein further comprises intracellular signaling domains. In some embodiments, the intracellular signaling domains may comprise at least one co-stimulatory signaling domain and at last one cytoplasmic signaling domain.

Exemplary co-stimulatory signaling domains may be derived from a suitable immune receptor, for example, OX40, CD70, CD27, CD28, CD5, ICAM-1, LFA-1 (CD11a/CD18), ICOS (CD278), DAP10, and DAP12. Hence, the CAR may have a co-stimulatory domain derived from 4-1BB, OX40, CD70, CD27, CD28, CD5, ICAM-1, LFA-1 (CD11a/CD18), ICOS (CD278), DAP10, and DAP12 or any combination thereof. In some examples, the co-stimulatory domain for use in the bi-specific CAR disclosed herein may be from co-stimulatory receptor 4-1BB (a.k.a., CD137), for example, from the human 4-1BB. An example is provided in the Sequence Table.

The intracellular signaling domains in the bi-specific CAR disclosed herein may also comprise one or more cytoplasmic signaling domains, e.g., a cytoplasmic signaling domain comprising an ITAM (e.g., ITAM1, ITAM2, and/or ITAM3 of CD3ζ such as those provided in the Sequence Table below). Examples include a CD3ζ signaling domain, an interleukin 2 receptor beta subunit (IL-2Rβ) cytoplasmic signaling domain, or a combination thereof. See amino acid sequences of exemplary CD3ζ signaling domain and a truncated IL-2Rβ cytoplasmic signaling domain in the Sequence Table, which can be used, either alone or combination, in the bi-specific antibodies disclosed herein. In some examples, the intracellular signaling domain may comprise a 4-1BB co-stimulatory domain and an ITAM motif from CD3ζ such as ITAM3 of CD3ζ.

### (c) Hinge and Transmembrane Domains

The bi-specific CAR polypeptide disclosed herein may contain a hinge and transmembrane domain located between the extracellular domain and the intracellular signaling domains. The hinge section can be any oligopeptide or polypeptide providing flexibility to the CAR, or domains thereof, or to prevent steric hindrance of the CAR, or domains thereof. In some embodiments, a hinge section may comprise up to 300 amino acids (e.g., 10 to 100 amino acids, or 5 to 20 amino acids). In some embodiments, one or more hinge domain(s) may be included in other regions of a CAR. In some examples, the hinge domain may be of CD28, CD8, an IgD or an IgG, such as IgG1 or IgG4. See U.S. Pat. No. 10,160,794. In one specific example, the hinge domain may be a CD8 hinge domain. Other hinge domains may be used.

The transmembrane section can be a hydrophobic alpha helix that spans the membrane. As used herein, a “transmembrane domain” refers to any protein structure that is thermodynamically stable in a cell membrane, preferably a eukaryotic cell membrane. The transmembrane domain can provide stability of the CAR containing such. In some embodiments, the transmembrane domain may be obtained from a suitable cell-surface receptor, for example, the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, CD271, TNFRSF19 and Killer Cell Immunoglobulin-Like Receptor (KIR). In some examples, the transmembrane domain can be a CD8 transmembrane domain.

An exemplary hinge and transmembrane domain of CD8 is provided in the Sequence Table, which can be used in constructing any of the bi-specific CARs disclosed herein.

### (d) Exemplary Bi-specific CARs

The bi-specific CARs for use in the present disclosure may contain one or more components provided in the Sequence Table, or a functional variant thereof. A functional variant of a reference CAR component listed in the Sequence Table (e.g., the antigen-binding moiety, the hinge/transmembrane domain, the co-stimulatory signaling domain, and the cytoplasmic signaling domain, etc.) may share at least 85% sequence identity (e.g., at least 90%, at least 95%, at least 97%, at least 98%, or higher) and preserves substantially the same functionality as the reference CAR component.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25 (17): 3389-3402, 1997. When utilizing BLAST and Gapped BLAST programs, the default parameters of the respective programs (e.g., XBLAST and NBLAST) can be used.

In some examples, the one or more components used in any of the bi-specific CAR may contain one or more conservative amino acid residue substitutions relative to the reference CAR components provided in the Sequence Table.

As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: ((a) A→G, S; (b) R→K, H; (c) N→Q, H; (d) D→E, N; (e) C→S, A; (f) Q→N; (g) E→D, Q; (h) G→A; (i) H→N, Q; (j) I→L, V; (k) L→I, V; (l) K→R, H; (m) M→L, I, Y; (n) F→Y, M, L; (o) P→A; (p) S→T; (q) T→S; (r) W→Y, F; (s) Y→W, F; and (t) V→I, L.

Examples of bi-specific CARs specific to Meso/BCMA, HER2/BCMA, GPC3/BCMA, CD19/BCMA, CD70/BCMA, Claudin 18.2/BCMA, PSMA/BCMA, and EGFR/BCMA are provided in the Sequence Table. Also provided in the Sequence Table is an exemplary two-chain bi-specific CAR, one chain binding to CD70 and the other chain binding to BCMA. Any of these examples, as well as their encoding nucleic acids and host cells expressing such, is within the scope of the present disclosure. In other instances, the two-chain bi-specific CAR (split bi-specific CAR) may comprise (a) one CAR polypeptide specific to HER2, Claudin 18.2, mesothelin, PSMA, GPC3, CD19, CD70, or EGFR provided in the Sequence Table (e.g., comprising either an scFv antigen binding moiety or a VHH antigen binding moiety), and (b) one CAR polypeptide specific to BCMA as also provided in the Sequence Table.

### (B) Genetically Engineered T Cells Expressing Modified T Cell Receptors and CAR

In some aspects, provided herein are genetically engineered T cells expressing aT cell receptor (TCR) (e.g., an engineered TCR) specific to a TAA and a CAR specific to either BCMA or CD19.

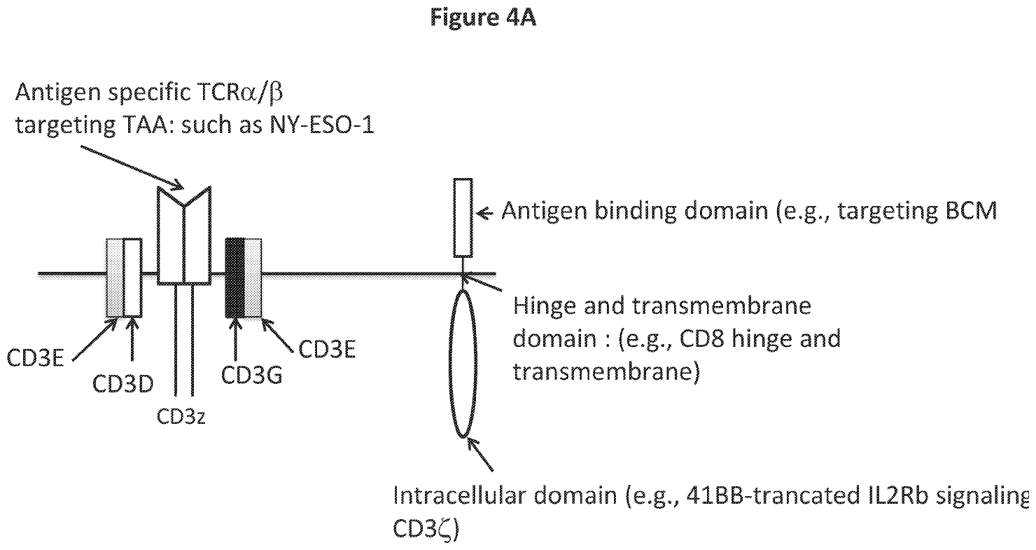

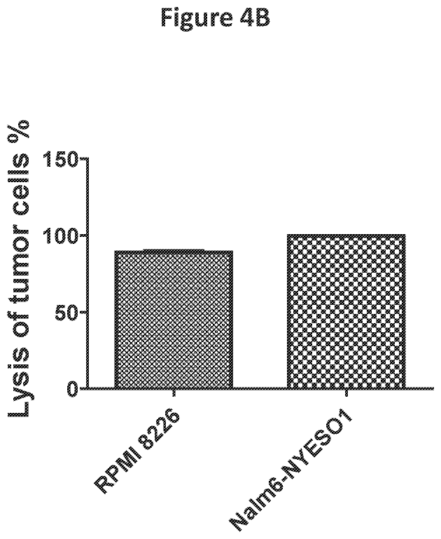

In some instances, the TCR comprises a TCRα chain and a TCRβ chain, which form a complex for recognizing a TAA or an antigenic peptide thereof presented by an MHC complex. See, e.g., FIG. 4A. An exemplary TCRα/TCRβ pair specific to NY-ESO-1 is provided in the Sequence Table. Such a TCR may be a native TCR molecule, for example, from a naturally-occurring T cell specific to a tumor antigen. Alternatively, the TCR may be an engineered one, to which the specificity to a TAA is grafted.

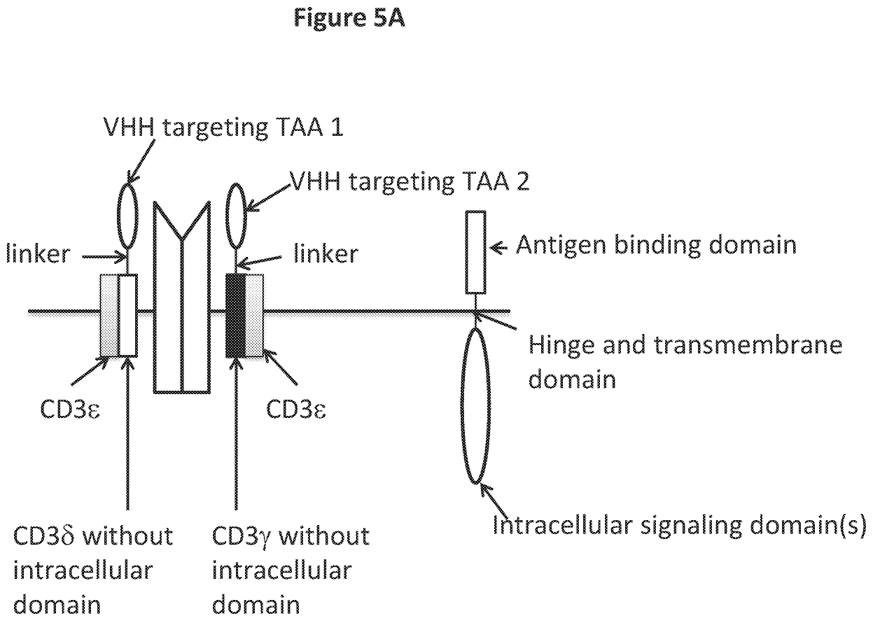

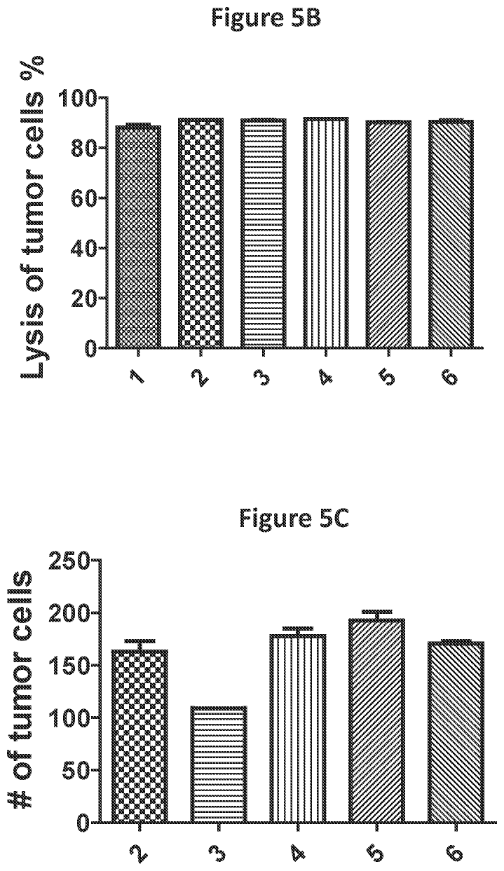

In other instances, the TCR comprises a TCRα chain and a TCRβ chain, which are in complex with CD3 complex comprising a modified CD3δ chain and a modified CD3γ chain. See, e.g., FIG. 5A. The modified CD3δ chain may comprise a truncated CD3δ chain without its intracellular domain fused to a first antigen binding moiety specific to a first TAA. The modified CD3γ chain may comprise a truncated CD3γ chain without its intracellular domain fused to a second antigen binding moiety specific to a second TAA. The first and second TAAs can be any of the TAAs known the art or disclosed herein. In some instances, the first TAA is different from the second TAA. In other instances, the first TAA is identical to the second TAA. Either the first antigen-binding moiety or the second antigen-binding moiety may be a scFv fragment. Alternatively, Either the first antigen-binding moiety or the second antigen-binding moiety may be a single-domain antibody, such as a VHH. In some examples, both the first antigen-binding moiety and the second antigen-binding moiety are VHH fragment.

In some examples, the first TAA is CD33 and the second TAA is CD123, or vice versa. The first antigen-binding moiety and/or the second antigen binding moiety may be VHH fragments. Examples of VHH fragments specific to CD33 or CD123 are provided in the Sequence Table, any of which can be used for making the modified TCR disclosed herein.

Any of the TAA-specific TCRs disclosed herein may be co-expressed with a CAR specific to CD19 or BCMA in genetically engineered T cells. See, e.g., FIGS. 4A and 5A. Any of the anti-CD19 or anti-BCMACARs disclosed herein can be used in such genetically engineered T cells. See examples in the Sequence Table.

### (C) Additional Modifications to Genetically Engineered T Cells

Any of the genetically engineered T cells expressing the bi-specific CAR or the TAA-specific TCR and a CAR as disclosed herein may be further engineered to express an antagonist of a cytokine, for example, an antagonist of a cytokine capable of activating immune responses. Antagonist as used herein refers to molecules capable of inhibiting or eliminating the bioactivity of the target cytokine to a meaningful degree, for example, by at least 20%, 50%, 70%, 85%, 90%, or above.

Exemplary target cytokines include, but are not limited to, interleukin-1 (IL-1), interleukin-1 (IL-2), interleukin-6 (IL-6), interleukin-7 (IL-7), interleukin-1 (IL-9), interleukin-10 (IL-10), interleukin-12 (IL-12), interleukin-15 (IL-15), interleukin-18 (IL-18), interleukin-21 (IL-21), interleukin-23 (IL-23), interleukin-24 (IL-24), interleukin-33 (IL-33), interleukin-36 (IL-36), GM-CSF, interferon gamma (IFNγ), and Chemokine (C-C motif) ligand 19 (CCL19).

In some embodiments, the antagonist of a target cytokine may be an antibody capable of binding to the target cytokine and inhibiting its bioactivity. A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also the Human Genome Mapping Project Resources at the Medical Research Council in the United Kingdom and the antibody rules described at the Bioinformatics and Computational Biology group website at University College London.

The antagonistic antibodies disclosed herein may be of any suitable format. An antibody (interchangeably used in plural form) as used herein is an immunoglobulin molecule capable of specific binding to a target cytokine as disclosed herein through at least one antigen recognition site, located in the variable region of the immunoglobulin molecule. As used herein, the term “antibody” encompasses not only intact (e.g., full-length) antibodies and heavy chain antibodies (e.g., an Alpaca heavy chain IgG antibody), but also antigen-binding fragments thereof (such as Fab, Fab′, F(ab′)2, Fv), single chain (scFv), single-domain antibody (sdAb; VHH), also known as a nanobody, mutants thereof, fusion proteins comprising an antibody portion, humanized antibodies, chimeric antibodies, diabodies, linear antibodies, single chain antibodies, multi-specific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

In some embodiments, the antibodies described herein that “bind” a target cytokine may specifically bind to the target cytokine. An antibody that “specifically binds” (used interchangeably herein) to a target or an epitope is a term well understood in the art, and methods to determine such specific binding are also well known in the art. A molecule is said to exhibit “specific binding” if it reacts or associates more frequently, more rapidly, with greater duration and/or with greater affinity with a particular target antigen than it does with alternative targets. An antibody “specifically binds” to a target cytokine if it binds with greater affinity, avidity, more readily, and/or with greater duration than it binds to other substances. For example, an antibody that specifically (or preferentially) binds to a target cytokine is an antibody that binds this cytokine with greater affinity, avidity, more readily, and/or with greater duration than it binds to other cytokine or other epitope in the target cytokine. It is also understood by reading this definition that, for example, an antibody that specifically binds to a first target cytokine may or may not specifically or preferentially bind to a second target cytokine. As such, “specific binding” or “preferential binding” does not necessarily require (although it can include) exclusive binding. Generally, but not necessarily, reference to binding means preferential binding.

In some embodiments, an antagonistic antibody of a target cytokine as described herein has a suitable binding affinity for the target cytokine or an antigenic epitope thereof. As used herein, “binding affinity” refers to the apparent association constant or KA. The KA is the reciprocal of the dissociation constant (KD). The antagonistic antibody described herein may have a binding affinity (KD) of at least 10⁻⁵, 10⁻⁶, 10⁻⁷, 10⁻⁸, 10⁻⁹, 10⁻¹⁰ M, or lower for the target cytokine or antigenic epitope thereof. An increased binding affinity corresponds to a decreased KD. Higher affinity binding of an antibody for a first target cytokine relative to a second target cytokine can be indicated by a higher KA (or a smaller numerical value KD) for binding the first target cytokine than the KA (or numerical value KD) for binding the second target cytokine. In such cases, the antibody has specificity for the first target cytokine relative to the second target cytokine. In some embodiments, the antagonistic antibodies described herein have a higher binding affinity (a higher KA or smaller KD) to the target cytokine in mature form as compared to the binding affinity to the target cytokine in precursor form or another protein, e.g., a cytokine in the same family as the target cytokine. Differences in binding affinity (e.g., for specificity or other comparisons) can be at least 1.5, 2, 3, 4, 5, 10, 15, 20, 37.5, 50, 70, 80, 91, 100, 500, 1000, 10,000 or 10⁵ fold.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

$$[ Bound ] = [ Free ] / ( Kd + [ Free ] )$$

It is not always necessary to make an exact determination of KA, though, since sometimes it is sufficient to obtain a quantitative measurement of affinity, e.g., determined using a method such as ELISA or FACS analysis, is proportional to KA, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

In some embodiments, the antagonistic antibody as described herein can bind and inhibit the signaling mediated by the target cytokine by at least 50% (e.g., 60%, 70%, 80%, 90%, 95% or greater). The inhibitory activity of an antagonistic antibody described herein can be determined by routine methods known in the art.

The antibodies described herein can be murine, rat, human, or any other origin (including chimeric or humanized antibodies). Such antibodies are non-naturally occurring, e.g., would not be produced in an animal without human act (e.g., immunizing such an animal with a desired antigen or fragment thereof).

Any of the antibodies described herein can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In one example, the antibody used in the methods described herein is a humanized antibody. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, and/or six), which are altered with respect to the original antibody, which are also termed one or more CDRs “derived from” one or more CDRs from the original antibody. Humanized antibodies may also involve affinity maturation.

In other embodiments, the antagonist of a target cytokine may comprise a binding moiety to the target cytokine, which may be fused to an immune activating cytokine (e.g., IL-2, IL-7, IL-9, IL-10, IL-12, IL-15, IL-18, IL-21, IL-23, IL-24, IL-36, IL-33, and CCL19). The binding moiety may be an antigen-binding fragment of an antibody specific to the target cytokine (e.g., an scFv fragment or a VHH fragment). Alternatively, the binding moiety may be a soluble receptor that binds the target cytokine.

In some examples, the fusion antagonist comprises a binding moiety to IFNγ fused to IL-18. The binding moiety to IFNγ (e.g., an antibody such as an scFv fragment that binds IFNγ) may be fused to the N-terminus of the IL-18. Alternatively, the binding moiety to IFNγ (e.g., an antibody such as an scFv fragment that binds IFNγ) may be fused to the C-terminus of the IL-18. In some instances, the binding moiety to IFNγ (e.g., an antibody such as an scFv fragment that binds IFNγ) and the IL-18 moiety may be linked via a peptide linker. Alternatively, these two moieties may be linked directly. One example of such a fusion protein is provided in the Sequence Table below.

### II. Antigen-Presenting Cells (APCs)

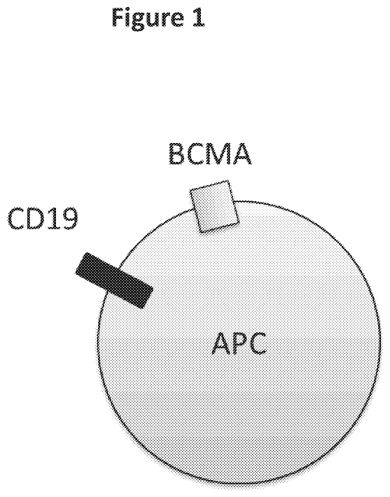

The present disclosure also provides a population of antigen-presenting cells (APCs) that express CD19 and/or BCMA. See, e.g., FIG. 1. Such APCs may also express a TAA such as those disclosed herein.

Antigen-presenting cells are cells that display antigens or antigenic peptides by major histocompatibility complex (MHC) molecules (MHC I or MHC II molecules) on cell surface for recognition by T cells. Typical APCs include immune cells such as immune cells, which optionally are T-cells, Natural Killer (NK) cells, tumor infiltrating lymphocytes, dendritic cells, macrophages, B cells, neutrophils, eosinophils, basophils, mast cells, myeloid-derived suppressor cells, and/or mesenchymal stem cells. Any of these immune cells or a combination thereof may be used in the present disclosure.

In some instances, the APCs disclosed herein may be genetically engineered to further express one or more membrane-bound stimulatory cytokines. Examples of such stimulatory cytokines include, but are not limited to mIL-10, mIL-18, mIL-15, mIL-9, and mIL-21. Amino acid sequences of such exemplary membrane-bound stimulatory cytokines are provided in the Sequence Table below.

In some embodiments, the APCs for use in the present disclosure may be naturally-occurring APC cells that express CD19 and/or BCMA, and optionally a TAA as well. Alternatively, the APCs may be genetically engineered to express one or more of the antigens of CD19, BCMA, and optionally the TAA.

In some examples, the APCs for use in the method disclosed herein may be universal APCs prepared from an NK cell line, for example, NK92-MI cells. For example, expression vectors carrying transgenes encoding CD19, BCMA, or the TAA may be introduced into the NK cells via conventional methods and the transfected cells expressing the target antigen may be isolated. In some instances, NK cells that stably expresses the target antigen can be established via conventional methodology.

In some examples, the APCs may be immune cells, for example, T-cells, Natural Killer (NK) cells, tumor infiltrating lymphocytes, dendritic cells, macrophages, B cells, neutrophils, eosinophils, basophils, mast cells, myeloid-derived suppressor cells.

In some examples, the APCs may be stem cells, for example, mesenchymal stem cells.

In some examples, the APCs may be tumor cells (e.g., a tumor cell line such as K562 cells).

### III. Methods of Preparing Genetically Engineered Immune Cells and APCs

The genetically engineered T cells and antigen-presenting cells may be prepared from immune cells, which can be derived from a suitable source. Examples include, but are not limited to, immune cell populations obtained from donors such as healthy human donors. In some examples, the immune cells may be derived from PBMCs. Alternatively, the immune cells may be derived from stem cells (e.g., adult stem cells, non-human embryonic stem cells, more particularly non-human stem cells, cord blood stem cells, progenitor cells, bone marrow stem cells, induced pluripotent stem cells, totipotent stem cells or hematopoietic stem cells). In some examples, the immune cells may be derived from the differentiation of a population of induced pluripotent cells (iPSCs).

Suitable immune cells include, but are not limited to, T-cells, NK cells, tumor infiltrating lymphocytes, dendritic cells, macrophages, B cells, neutrophils, eosinophils, basophils, mast cells, myeloid-derived suppressor cells, mesenchymal stem cells, precursors thereof, or combinations thereof. The T-cells may be selected from the group consisting of inflammatory T-lymphocytes, cytotoxic T-lymphocytes, regulatory T-lymphocytes or helper T-lymphocytes. In some embodiments, the T-cells can be derived from the group consisting of CD4+ T-lymphocytes and CD8+ T-lymphocytes.

Any of the genetic modifications disclosed herein, including knock-in transgenes encoding any of the bi-specific CAR, the modified TCR, and/or the cytokine antagonists disclosed herein, may be introduced into suitable immune cells by routine methods and/or approaches described herein. Typically, such methods would involve delivery of genetic material into the suitable immune cells to either down-regulate expression of a target endogenous inflammatory protein, express a cytokine antagonist of interest or express an immune suppressive cytokine of interest.

To generate a knock-in of one or more bi-specific CAR, modified TCR, and cytokine antagonists described herein, a coding sequence of any of the bi-specific CARs, modified TCRs, and cytokine antagonists described herein may be cloned into a suitable expression vector (e.g., including but not limited to lentiviral vectors, retroviral vectors, adenoviral vectors, adeno-associated vectors, PiggyBac transposon vector and Sleeping Beauty transposon vector) and introduced into host immune cells using conventional recombinant technology. Sambrook et al., Molecular Cloning, A Laboratory Mannual, 3rd Ed., Cold Spring Harbor Laboratory Press. As a result, modified immune cells of the present disclosure may comprise one or more exogenous nucleic acids encoding at least one bi-specific CAR or a chain thereof, at least one modified TCR, and/or at least one cytokine antagonist. In some instances, the one or more transgenes may be integrated into the genome of the cell for stable expression. In some instances, the transgenes may not be integrated into the genome of the cell.

An exogenous nucleic acid comprising a coding sequence of a bi-specific CAR or a chain thereof, a modified TCR, and/or a cytokine antagonist may further comprise a suitable promoter, which can be in operable linkage to the coding sequence. A promoter, as used herein, refers to a nucleotide sequence (site) on a nucleic acid to which RNA polymerase can bind to initiate the transcription of the coding DNA (e.g., for a cytokine antagonist) into mRNA, which will then be translated into the corresponding protein (i.e., expression of a gene). A promoter is considered to be “operably linked” to a coding sequence when it is in a correct functional location and orientation relative to the coding sequence to control (“drive”) transcriptional initiation and expression of that coding sequence (to produce the corresponding protein molecules). In some instances, the promoter described herein can be constitutive, which initiates transcription independent other regulatory factors. In some instances, the promoter described herein can be inducible, which is dependent on regulatory factors for transcription. Exemplary promoters include, but are not limited to ubiquitin, RSV, CMV, EF1α and PGK1. In one example, one or more nucleic acids encoding one or more antagonists of one or more inflammatory cytokines as those described herein, operably linked to one or more suitable promoters can be introduced into immune cells via conventional methods to drive expression of one or more antagonists.

Additionally, the exogenous nucleic acids described herein may further contain, for example, one or more of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; versatile multiple cloning sites; and T7 and SP6 RNA promoters for in vitro transcription of sense and antisense RNA. Suitable methods for producing vectors containing transgenes are well known and available in the art. Sambrook et al., Molecular Cloning, A Laboratory Mannual, 3rd Ed., Cold Spring Harbor Laboratory Press.

In some instances, a combination of bi-specific CAR or chains thereof, modified TCRs, and/or cytokine antagonists as described herein can be constructed in one expression cassette in a multicistronic manner such that the multiple cytokine antagonists as separate polypeptides. In some examples, an internal ribosome entry site can be inserted between two coding sequences to achieve this goal. Alternatively, a nucleotide sequence coding for a self-cleaving peptide (e.g., T2A or P2A) can be inserted between two coding sequences. Exemplary designs of such multicistronic expression cassettes are provided in the Sequence Table below.

A population of immune cells comprising any of the modified immune cells described herein, or a combination thereof, may be prepared by introducing into a population of host immune cells one or more of the knock-in modifications disclosed herein.

In some instances, one or more modifications are introduced into the host cells in a sequential manner without isolation and/or enrichment of modified cells after a preceding modification event and prior to the next modification event. In that case, the resultant immune cell population may be heterogeneous, comprising cells harboring different modifications or different combination of modifications. Such an immune cell population may also comprise unmodified immune cells. The level of each modification event occurring in the immune cell population can be controlled by the amount of genetic materials that induce such modification as relative to the total number of the host immune cells. See also above discussions.

In other instances, modified immune cells may be isolated and enriched after a first modification event before performing a second modification event. This approach would result in the production of a substantially homogenous immune cell population harboring all of the knock-in and/or knock-out modifications introduced into the cells.

### IV. Therapeutic Applications

Any of the genetically engineered T cells described herein may be used in an adoptive immune cell therapy for treating a target disease, such as a solid tumor or a hematopoietic cancer (e.g., chronic lymphocytic leukemia or CLL), together with any of the APCs disclosed herein to enhance treatment efficacy. In some instances, the methods provided herein may also be used for treating immune disorders such as autoimmune disorders (e.g., systemic lupus erythematosus when the genetically engineered T cells express an anti-CD19 CAR or an anti-BCMA CAR), or for treating an infectious disease (e.g., using genetically engineered T cells expressing one or more CARs targeting one or more antigens derived from a pathogen such as a virus or a bacterium).

To practice the therapeutic methods described herein, an effective amount of the genetically engineered T cells as disclosed herein may be administered to a subject who needs treatment via a suitable route (e.g., intravenous infusion), concurrently with an effective amount of the APCs The genetically engineered T cells and/or the APCs may be mixed with a pharmaceutically acceptable carrier to form a pharmaceutical composition prior to administration, which is also within the scope of the present disclosure.

The term “an effective amount” as used herein refers to the amount of each active agent required to confer therapeutic effect on the subject, either alone or in combination with one or more active agents. Effective amounts vary, as recognized by those skilled in the art, depending on the particular condition being treated, the severity of the condition, individual patient parameters including age, physical condition, size, gender and weight, the duration of treatment, route of administration, excipient usage, co-usage (if any) with other active agents and like factors within the knowledge and expertise of the health practitioner. The quantity to be administered depends on the subject to be treated, including, for example, the capacity of the individual's immune system to produce a cell-mediated immune response. Precise mounts of active ingredient required to be administered depend on the judgment of the practitioner. However, suitable dosage ranges are readily determinable by one skilled in the art.

The term “treating” as used herein refers to the application or administration of a composition including one or more active agents to a subject, who has a target disease, a symptom of the target disease, or a predisposition toward the target disease, with the purpose to cure, heal, alleviate, relieve, alter, remedy, ameliorate, improve, or affect the disease, the symptoms of the disease, or the predisposition toward the disease.

The genetically engineered T cells, the APCs, or both may be autologous to the subject, i.e., the cells are obtained from the subject in need of the treatment, modified to express one or more cytokine antagonists described herein, to express a CAR construct and/or exogenous TCR, or a combination thereof, or to express the antigen of CD19 and/or BCMA The resultant modified cells can then be administered to the same subject. Administration of autologous cells to a subject may result in reduced rejection of the donor cells as compared to administration of non-autologous cells.

Alternatively, the genetically engineered T cells, the APCs, or both can be allogeneic cells, i.e., the cells are obtained from a first subject, modified as described herein and administered to a second subject that is different from the first subject but of the same species. For example, allogeneic immune cells may be derived from a human donor and administered to a human recipient who is different from the donor.

The subject to be treated may be a mammal (e.g., human, mouse, pig, cow, rat, dog, guinea pig, rabbit, hamster, cat, goat, sheep or monkey). The subject may be suffering from cancer, for example, a solid tumor or a hematopoietic cancer such as AML. In some instances, the subject has a cancer involving cancer cells expressing a target antigen, e.g., CD19, BCMA, HER2, mesothelin, GPC3, claudin 18.2, PSMA, CD70, and/or EGFR. Corresponding CAR-T cells may be selected based on expression of target antigens in the subject for treatment.

In some examples, genetically engineered T cells expressing a bi-specific CAR capable of binding to CD19 or BCMA, and a TAA such as HER2, mesothelin, GPC3, claudin 18.2, PSMA, CD70, or EGFR may be co-used with APCs expressing CD19 and/or BCMA, and optionally the TAA for treating a solid tumor.

In other examples, genetically engineered T cells expressing a TAA-specific TCR (e.g., specific to NY-ESO-1) and a CAR specific to BCMA can be co-used with APCs expressing BCMA and/or CD19, and optionally the TAA for treating a solid tumor.

In some examples, genetically engineered T cells expressing a bi-specific CAR capable of binding to CD70 and BCMA may be co-used with APCs expressing BCMA and/or CD19, and optionally CD70 for treating AML.

Alternatively, genetically engineered T cells expressing a modified TCR capable of binding to CD33 and CD123 and a CAR specific to BCMA may be co-used with APCs expressing BCMA and/or CD19, optionally CD33 and/or CD123 for treating AML.

An effective amount of the genetically engineered T cells and APCs may be administered to a human patient in need of the treatment via a suitable route, e.g., intravenous infusion. In some instances, about 1×10⁶ to about 1×10⁸ CAR+ T cells and/or APC cells may be given to a human patient (e.g., a leukemia patient, a lymphoma patient, or a multiple myeloma patient). In some examples, a human patient may receive multiple doses of the genetically engineered immune cells. For example, the patient may receive two doses of the immune cells on two consecutive days. In some instances, the first dose is the same as the second dose. In other instances, the first dose is lower than the second dose, or vice versa.

The CAR-T cell/APC combined therapy disclosed herein may be utilized in conjunction with other types of therapy for cancer, such as chemotherapy, surgery, radiation, gene therapy, and so forth. Such therapies can be administered simultaneously or sequentially (in any order) with the immunotherapy described herein. When co-administered with an additional therapeutic agent, suitable therapeutically effective dosages for each agent may be lowered due to the additive action or synergy.

In some examples, the subject is subject to a suitable anti-cancer therapy (e.g., those disclosed herein) to reduce tumor burden prior to the CAR-T/APC therapy disclosed herein. For example, the subject (e.g., a human cancer patient) may be subject to a chemotherapy (e.g., comprising a single chemotherapeutic agent or a combination of two or more chemotherapeutic agents) at a dose that substantially reduces tumor burden. In some instances, the chemotherapy may reduce the total white blood cell count in the subject to lower than 10⁸/L, e.g., lower than 10⁷/L. Tumor burden of a patient after the initial anti-cancer therapy, and/or after the CAR-T cell/APC therapy disclosed herein may be monitored via routine methods. If a patient showed a high growth rate of cancer cells after the initial anti-cancer therapy and/or after the CAR-T/APC therapy, the patient may be subject to a new round of chemotherapy to reduce tumor burden followed by any of the CAR-T therapy as disclosed herein.

Non-limiting examples of other anti-cancer therapeutic agents useful for combination with the modified immune cells described herein include, but are not limited to, immune checkpoint inhibitors (e.g., PDL1, PD1, and CTLA4 inhibitors), anti-angiogenic agents (e.g., TNP-470, platelet factor 4, thrombospondin-1, tissue inhibitors of metalloproteases, prolactin, angiostatin, endostatin, bFGF soluble receptor, transforming growth factor beta, interferon alpha, soluble KDR and FLT-1 receptors, and placental proliferin-related protein); a VEGF antagonist (e.g., anti-VEGF antibodies, VEGF variants, soluble VEGF receptor fragments); chemotherapeutic compounds. Exemplary chemotherapeutic compounds include pyrimidine analogs (e.g., 5-fluorouracil, floxuridine, capecitabine, gemcitabine and cytarabine); purine analogs (e.g., fludarabine); folate antagonists (e.g., mercaptopurine and thioguanine); antiproliferative or antimitotic agents, for example, vinca alkaloids; microtubule disruptors such as taxane (e.g., paclitaxel, docetaxel), vincristin, vinblastin, nocodazole, epothilones and navelbine, and epidipodophyllotoxins; DNA damaging agents (e.g., actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide).

In some embodiments, radiation or radiation and chemotherapy are used in combination with the cell populations comprising modified immune cells described herein. Additional useful agents and therapies can be found in Physician's Desk Reference, 59.sup.th edition, (2005), Thomson P D R, Montvale N.J.; Gennaro et al., Eds. Remington's The Science and Practice of Pharmacy 20.sup.th edition, (2000), Lippincott Williams and Wilkins, Baltimore Md.; Braunwald et al., Eds. Harrison's Principles of Internal Medicine, 15.sup.th edition, (2001), McGraw Hill, NY; Berkow et al., Eds. The Merck Manual of Diagnosis and Therapy, (1992), Merck Research Laboratories, Rahway N.J.

### V. Kits for Therapeutic Uses or Making Modified Immune Cells

The present disclosure also provides kits for use of any of the target diseases described herein involving the genetically engineered T cells and APCs described herein and kits for use in making the modified immune cells as described herein.

A kit for therapeutic use as described herein may include one or more containers comprising a population of the genetically engineered T cells or a population of the APCs, each of which may be formulated to form a pharmaceutical composition. In some embodiments, the kit can additionally comprise instructions for use of the genetically engineered T cells and APCs in any of the methods described herein. The included instructions may comprise a description of administration of the cell populations or a pharmaceutical composition comprising such to a subject to achieve the intended activity in a subject. The kit may further comprise a description of selecting a subject suitable for treatment based on identifying whether the subject is in need of the treatment. In some embodiments, the instructions comprise a description of administering the genetically engineered T cells and APCs or the pharmaceutical composition comprising such to a subject who is in need of the treatment.

The instructions relating to the use of the genetically engineered T cells and APCs or the pharmaceutical composition comprising such as described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the pharmaceutical composition is a population of genetically engineered T cells and APCs.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

Also provided here are kits for use in making the modified immune cells or APCs as described herein. Such a kit may include one or more containers each containing reagents for use in introducing the knock-in modifications into immune cells or APCs. Alternatively or in addition, the kit may comprise one or more exogenous nucleic acids for expressing any of the bi-specific CARs, TCRs, and/or cytokine antagonists as also described herein and reagents for delivering the exogenous nucleic acids into host immune cells. Such a kit may further include instructions for making the desired modifications to host immune cells.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introuction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Preparation of Universal Antigen-Presenting Cells (APCs) Expressing CD19 and BCMA

NK92-MI cells are cultured under conventional conditions. A lentiviral expression vector comprising coding sequences for BCMA and CD19 is constructed via recombinant technology in bicistronic format, in which the BCMA gene and the CD19 gene are connected by a nucleotide sequence encoding a T2A peptide. FIG. 1. The amino acid sequences for BCMA, CD19, and T2A are provided in the Sequence Table below. The lentiviral expression vector is introduced into the NK92-MI cells and the NK92-MI cells expressing both the BCMA and CD19 surface proteins are enriched and can be used as universal APCs to enhance CAR-T cell efficacy.

### Example 2: Preparation and Characterization of Genetically Engineered CAR-T Cells for Solid Tumor Treatment

Lentiviral vectors designed for expressing a bi-specific CAR are constructed by the conventional recombinant technology. The bi-specific CAR comprises a first scFv that binds CD19, HER2, Mesothelin, GPC3, or Claudin 18.2, and a second scFv that binds BCMA In addition to the antigen binding moieties, each of the bi-specific CARs also contains a hinge and transmembrane domain of CD8, and intracellular signaling domains of 4-1BB-truncated IL2Rb signaling-CD3ζ. The amino acid sequences of the Meso/BCMA, GPC3/BCMA, HER2/BCMA, and Claudin 18.2/BCMA are provided in the Sequence Table below. See also FIG. 2A.

Primary T cells from healthy donors were activated by anti-CD3/CD28 beads (Thermo scientific). One day later, the T cells were transduced with a lentiviral vector encoding one of the above-noted bi-specific CARs. The transduced cells were expanded and tested for CD3 expression by FACS analysis and CD3+ population is gated for further analysis. Expression of the bi-specific CAR can be analyzed via conventional methods. For example, CAR expression was analyzed by flowcytometry using a biotinylated primary antibody recognizing the antibody fragment in the CAR and fluorescence-labeled Streptavidin.

Functionality of the bi-specific CAR-T cells is analyzed by coculture of the CAR-T cells with target APCs or target tumor cells to evaluate CAR-T cell proliferation and/or cytotoxicity. More specifically, the genetically engineered T cells expressing one of the bi-specific CARs noted above (see also Sequence Table and FIG. 2A) and co-expressing anti-IFNG scFv (see also Sequence Table) were cocultured at 1:3 ratio (E:T) with GFP expressing target cells MM1S and TAA expressing target cells to evaluate the killing activity. Different TAA targeting VHHs as indicated in the bispecific anti-TAA/BCMA CAR were tested. In FIG. 2B, HER2 and 2D3 refer to VHHs targeting TAA HER2; 182-19 and 182-6 refer to VHHs targeting TAA Claudin 18.2; Meso2 and Meso1 refer to VHHs targeting TAA mesothelin; PSMA363 and PSMA362 refer to VHHs targeting TAA PSMA; GPC3 refers to VHH targeting TAA GPC3; EGFR refers to VHH targeting TAA EGFR. In FIG. 2C, SKOV3 refers to HER2+ tumor targets; AGS-Claudin 18.2 refers to AGS cells expressing Claudin 18.2; Aspc1 refers to mesothelin+ tumor cells; LnCap refers to PSMA+ tumor cells; Huh7 refers to GPC3+ tumor cells; Panc1 refers to EGFR+ tumor cells. 3 days later, remaining GFP+ tumor cells were counted by flowcytometry to determine the percentage of tumor cells killed by CART cells. The results indicated that each of the bispecific anti TAA/BCMA CAR showed effective killing against BCMA+ MMIS cells, and effective killing against the corresponding TAA expressing tumor cells. FIGS. 2B and 2C.

### Example 3: Preparation of Genetically Engineered CAR-T Cells for Treatment of AML

Lentiviral vectors designed for expressing a bi-specific CAR are constructed by the conventional recombinant technology. The bi-specific CAR comprises two separate polypeptides, each comprising a binding moiety to an antigen of interest. The first polypeptide comprises an extracellular domain of CD27 (truncated CD27 without the intracellular domain) fused to a CD3ζ intracellular signaling domain. CAR1 (binds to CD70, a.k.a., anti-CD70 CAR) depicted in FIG. 3A. The second polypeptide comprises an anti-BCMA scFv, a hinge and transmembrane domain of CD8, and intracellular signaling domains of 4-1BB-truncated IL2Rb signaling-CD3ζ (SEQ ID NO:38). See CAR2 depicted in FIG. 3A.

The amino acid sequences of CAR1 and CAR2 are provided in the Sequence Table below. The coding sequences of CAR1 and CAR2 are in bi-cistronic format in the lentiviral vector, which are connected by a nucleotide sequence encoding a T2A peptide linker.

Primary T cells from healthy donors were activated by anti-CD3/CD28 beads (Thermo scientific). One day later, the T cells were transduced with the lentiviral vector encoding the two-chain bi-specific CAR described above. The transduced cells were expanded and tested for CD3 expression by FACS analysis and CD3+ population is gated for further analysis. Expression of the bi-specific CAR can be analyzed via conventional methods.

The genetically engineered T cells expressing the bispecific anti CD70/BCMA CAR described above (see also FIG. 3B) were cocultured at 1:3 ratio (E:T) with GFP expressing target cells RPMI8226, U937 and Molm13 to evaluate their target cell-killing activity. 3 days later, remaining GFP+ tumor cells were counted by flowcytometry to determine the percentage of tumor cells killed by the genetically engineered CAR-T cells. The results indicated that the bispecific anti CD70/BCMA CAR-T cells showed effective killing against BCMA+ RMPI8226 cells, CD123+U937 cells and CD123+/CD33+ Molm13 cells. FIG. 3B.

### Example 4: Preparation of Genetically Engineered TCR-T Cells for Treatment of Cancer Treatment

Lentiviral vectors designed for expressing a TCR receptor specific to a tumor antigen NY-ESO-1 and a CAR specific to BCMA (SEQ ID NO: 38) are constructed by the conventional recombinant technology. See FIG. 4A. The TCR specific to NY-ESO-1 contains a TCRα chain and a TCRβ chain, the amino acid sequences of which are provided in the Sequence Table below. The coding sequences for the two chains are in bi-cistronic format in the lentiviral vector, which are connected by a nucleotide sequence encoding a T2A peptide linker. The amino acid sequence of the anti-BCMA CAR is also provided in the Sequence Table. The coding sequence for the anti-BCMA CAR is connected to the coding sequence of the TCRβ chain by a nucleotide sequence encoding a P2A peptide linker.

Primary T cells from healthy donors were activated by anti-CD3/CD28 beads (Thermo scientific). One day later, the T cells were transduced with the lentiviral vector encoding the TCR and anti-BCMA CAR described above. The transduced cells were expanded and tested for CD3 expression by FACS analysis and CD3+ population was gated for further analysis. Expression of the bi-specific CAR was analyzed via conventional methods.

Genetically engineered T cells expressing the bispecific anti-TAA TCR/BCMA CAR as described above (see also Sequence Table and FIG. 4A) were cocultured at 1:3 ratio (E:T) with GFP expressing target cells RPMI8226, and Nalm6-NYESO1 to evaluate the killing activity. 3 days later, remaining GFP+ tumor cells were counted by flowcytometry to determine the percentage of tumor cells killed by CART cells. The results as shown in FIG. 4B indicated that anti bispecific anti TAA TCR/BCMA CAR showed effective killing against BCMA+ RMPI8226 cells and Nalm6-NYESO1.

### Example 5: Preparation and Characterization of Genetically Engineered T Cells Expressing a Bi-Specific TCR and an Anti-BCMA CAR for Treatment of AML

Lentiviral vectors designed for expressing a bi-specific TCR receptor specific to CD33 (e.g., TAA1 in FIG. 5A) and CD123 (e.g., TAA2 in FIG. 5A) and a CAR specific to BCMA (SEQ ID NO: 125) are constructed by the conventional recombinant technology. See FIG. 5A. The bi-specific TCR contains a first polypeptide comprising a VHH specific to CD33 fused to a CD3δ fragment (without the intracellular domain) and a second polypeptide comprising a VHH specific to CD123 fused to a CD3γ fragment (without the intracellular domain). The amino acid sequences of the anti-CD33 VHH and anti-CD123 VHH, as well as the CD3δ and CD3γ fragments, are provided in the Sequence Table. The coding sequences for the two polypeptides are connected via a nucleotide sequence encoding the T2A peptide linker. The amino acid sequence of the anti-BCMA CAR (SEQ ID NO: 125) is also provided in the Sequence Table. The coding sequence for the anti-BCMA CAR is connected to the coding sequence of one of the polypeptides by a nucleotide sequence encoding the P2A peptide linker.

Primary T cells from healthy donors were activated by anti-CD3/CD28 beads (Thermo scientific). One day later, the T cells were transduced with the lentiviral vector encoding the bi-specific TCR complex and anti-BCMA CAR described above. The transduced cells were expanded and tested for CD3 expression by FACS analysis and CD3+ population was gated for further analysis. Expression of the bi-specific CAR can be analyzed via conventional methods.

The genetically engineered T cells expressing the bi-specific TCR complex and the anti BCMA CAR described above (see also FIG. 5A) were cocultured at 1:3 ratio (E:T) with GFP expressing target cells RPMI8226 and Molm13 to evaluate the killing activity. 3 days later, remaining GFP+ tumor cells were counted by flowcytometry to determine the percentage of tumor cells killed by the genetically engineered T cells. The results indicated that anti CD123/CD33 CAR (with each of the 6 anti-CD123 VHH candidates listed in the Sequence Table) showed similar killing efficiency against BCMA+ RMPI8226 cells. FIG. 5B. Genetically engineered T cells expressing the bi-specific TCR with anti CD123 VHH3 showed relatively higher killing efficiency against CD123+/CD33+ Molm13 cells relative to other anti-CD123 VHH-containing bi-specific TCRs. FIG. 5C.

### Example 6: Co-Culture with Antigen-Presenting Cells Enhances CAR-T Cell Expansion

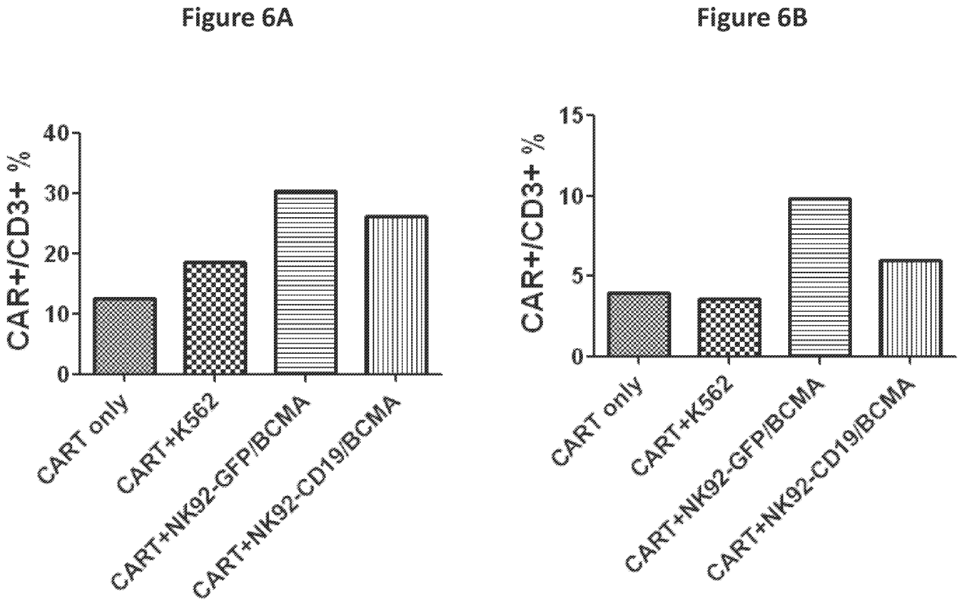

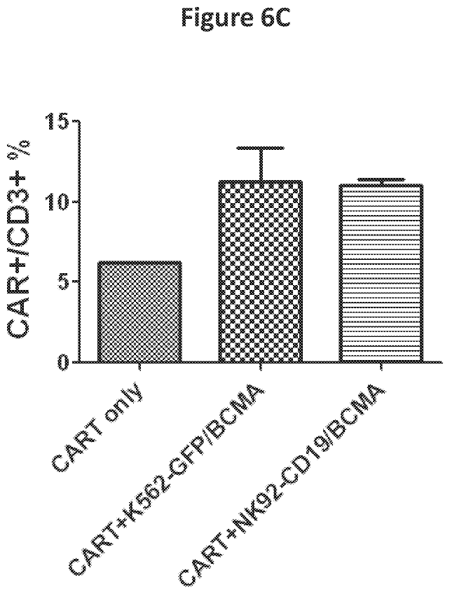

Genetically engineered T cells expressing the bi-specific CAR targeting CD70 and BCMA as disclosed in Example 3 above, expressing the anti-NY-ESO-1 TCR/anti-BCMA CAR as disclosed in Example 4 above, and expressing the bispecific anti-CD19 VHH/anti-BCMA CAR as disclosed in Example 2 above were cocultured with K562 cells, or K562 cells expressing GFP/BCMA, NK92 cells, NK92 cells expressing GFP/BCMA, or NK92 cells expressing CD19/BCMA to evaluate the effect of APC cells on CAR-T cell expansion. 3 days later, CAR+ cells were analyzed by flowcytometry, and the results shown in FIGS. 6A-6C indicated that co-culture with the CD19 and/or BCMA-expressing APC cells effectively enhanced expansion of all of the genetically engineered T cells tested herein.

### Example 7: In Vivo Anti-Tumor Efficacy of CAR-T Cells Co-Administered with Antigen-Presenting Cells

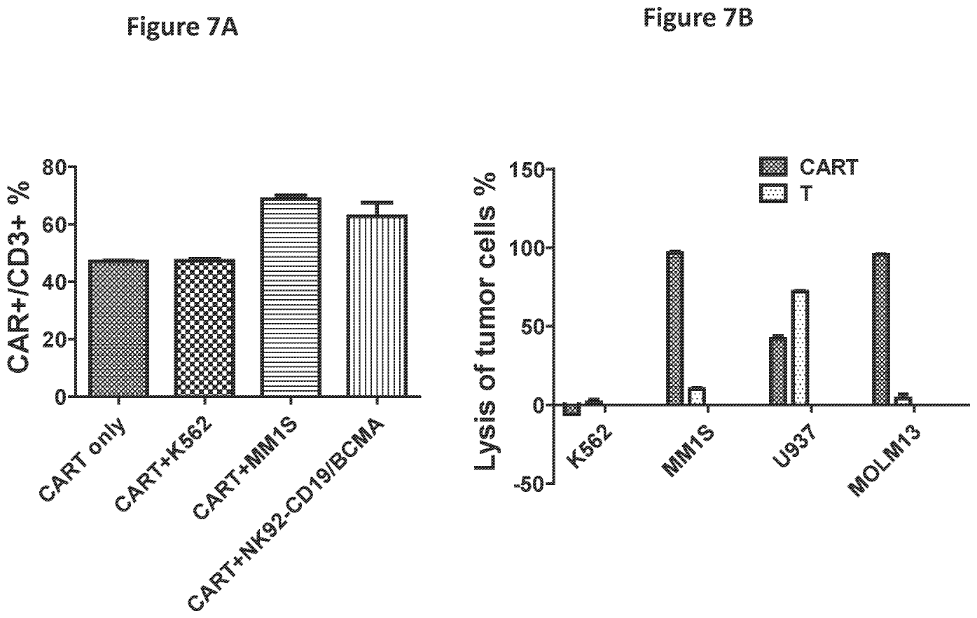

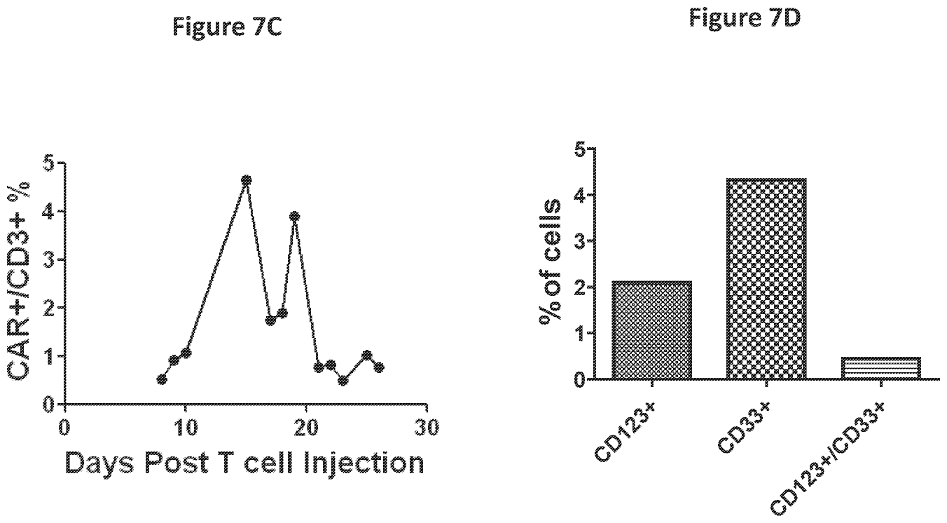

Genetically engineered T cells co-expressing TCR-based bi-specific CAR targeting CD123 (anti-CD123 VHH3) and CD33 and a CAR construct targeting BCMA (SEQ ID NO: 125) were prepared following the disclosures in Example 5 above. Structural information of the TCR-based bi-specific CAR and the anti-BCMA CAR (containing a 4-1BB costimulatory signaling domain and CD3z-ITAM3 signaling domain) is provided in the Sequence Table below. See also FIG. 5A. The CAR-T cells were cocultured with targets cells K562, MM1S or NK-92 expressing CD19 and BCMA. 3 days later, CAR percentage was analyzed by flowcytometry. As shown in FIG. 7A, effective CAR-T cell expansion was observed when co-cultured with BCMA+ MMIS cells or NK-92 expressing CD19 and BCMA, as compared with the co-culture with the K562 control cells.

The CAR-T cells were cocultured with GFP expressing targets cells K562, MMIS, CD123+U937 or CD123+/CD33+ Molm13 target cells at 1:3 ratio (E:T). 3 days later, remaining GFP+ tumor cells were counted by flowcytometry to determine the percentage of tumor cells killed by CART cells. As shown in FIG. 7B, anti CD123/CD33 CAR with #3 anti-CD123 VHH candidate showed effective killing against BCMA+MM1S and CD123+/CD33+ Molm13 cells. Surprisingly, the CART cells did not exhibit effective killing against CD123+U937 cells as compared to the non-transduced mock T cells, suggesting that the TCR based bispecific anti CD123/CD33 CAR is more robust in killing double positive CD123+/CD33+ targets than the single positive CD123 targets.

The CAR-T cells and CD19 and/or BCMA-expressing APC cells were used to treat a patient with relapsed/refractory AML. The patient was infused with 0.6×10⁸ of the CAR-T cells on Day0 and infused with 1.2×10⁸ NK92-CD19/BCMA cells on Day7. A 4.65% change of CAR percentage was observed on Day 15, which indicates expansion of the CAR-T cells in vivo. Since the TCR-based bispecific anti CD123/CD33 CAR lacks a costimulatory signaling, the expansion might be attributed to the co-expressed anti BCMA CAR stimulated by the APC NK-92-CD19/BCMA. FIG. 7C. In sum, the results show that in vivo CAR-T expansion could be enhanced by co-administration of APCs expressing BCMA.

17 days after CART infusion, the bone marrow cells were analyzed by flowcytometry. The results show low levels of CD123+ cells (2.1%) and CD33+ cells (4.32%) and a much lower level of CD123+/CD33+ double positive cells (0.45%). FIG. 7D. These results indicate that the TCR-based bispecific anti CD123/CD33 CAR was more effective in killing CD123+/CD33+ double positive cells, as compared with CD123+ or CD33+ single positive cells. This result suggests a superior safety feature in treatment of AML by CAR-T therapy.

Sequence Table

| | | | SEQ |
|---|---|---|---|
| | | | ID |
| Description | | Sequence | NO: |
| hCD19 | | MPPPRLLFFLLFLTPMEVRPEEPLVVKVEEGDNAVLQCLKGTSDGPTQQL | 1 |
| | | TWSRESPLKPFLKLSLGLPGLGIHMRPLAIWLFIFNVSQQMGGFYLCQPG | |
| | | PPSEKAWQPGWTVNVEGSGELFRWNVSDLGGLGCGLKNRSSEGPSSPSGK | |
| | | LMSPKLYVWAKDRPEIWEGEPPCLPPRDSLNQSLSQDLTMAPGSTLWLSC | |
| | | GVPPDSVSRGPLSWTHVHPKGPKSLLSLELKDDRPARDMWVMETGLLLPR | |
| | | ATAQDAGKYYCHRGNLTMSFHLEITARPVLWHWLLRTGGWKVSAVTLAYL | |
| | | IFCLCSLVGILHLQRALVLRRKRKRMTDPTRRFFKVTPPPGSGPQNQYGN | |
| | | VLSLPTPTSGLGRAQRWAAGLGGTAPSYGNPSSDVQADGALGSRSPPGVG | |
| | | PEEEEGEGYEEPDSEEDSEFYENDSNLGQDQLSQDGSGYENPEDEPLGPE | |
| | | DEDSFSNAESYENEDEELTQPVARTMDFLSPHGSAWDPSREATSLGSQSY | |
| | | EDMRGILYAAPQLRSIRGQPGPNHEEDADSYENMDNPDGPDPAWGGGGRM | |
| | | GTWSTR | |
| T2A | | GSGEGRGSLLTCGDVEENPGP | 2 |
| P2A | | GSGATNFSLLKQAGDVEENPGP | 3 |
| hBCMA | | MLQMAGQCSQNEYFDSLLHACIPCQLRCSSNTPPLTCQRYCNASVTNSVK | 4 |
| | | GTNAILWTCLGLSLIISLAVFVLMFLLRKINSEPLKDEFKNTGSGLLGMA | |
| | | NIDLEKSRTGDEIILPRGLEYTVEECTCEDCIKSKPKVDSDHCFPLPAME | |
| | | EGATILVTTKTNDYCKSLPAALSATEIEKSISAR | |
| CD8 Leader | | MALPVTALLLPLALLLHAARP | 5 |
| Linker A | | SGGGSDPGGGGSGGGGSPAG | 6 |
| Linker B | | GGGGSPAG | 63 |
| Linker C | | SGGGSGGSGGGSGGSDPAG | 64 |
| CD8 hinge and | | TTTPAPRPPTPAPTIASQPLSLRPEACRPAAGGAVHTRGLDFACDIYIWA | 7 |
| TM | | PLAGTCGVLLLSLVITLYC | |
| 4-1BB truncated | | KRGRKKLLYIFKQPFMRPVQTTQEEDGCSCRFPEEEEGGCEL | 8 |
| IL2 Rb | | NCRNTGPWLKKVLKCNTPDPSKFFSQLSSEHGGDVQKWLSSPFPSSSFSP | 9 |
| (truncated) | | GGLAPEISPLEVLERDKVTQLLPLNTDAYLSLQELQGQDPTHLV | |
| CD3z | | RVKFSRSADAPAYKQGQNQLYNELNLGRREEYDVLDKRRGRDPEMGGKPR | 10 |
| | | RKNPQEGLYNELQKDKMAEAYSEIGMKGERRRGKGHDGLYQGLSTATKDT | |
| | | YDAYRHQALPPR | |
| CD3z ITAM1 | | RVKFSRSADAPAYKQGQNQLYNELNLGRREEYDVLDKRRGRDPEMGGKPR | 65 |
| | | RKNPQ | |
| CD3z ITAM2 | | DKRRGRDPEMGGKPRRKNPQEGLYNELQKDKMAEAYSEIGMKGERRRGKG | 66 |
| | | H | |
| CD3z ITAM3 | | GMKGERRRGKGHDGLYQGLSTATKDTYDALHMQALPPR | 67 |
| Anti- | VH | QVQLVQSGAEVEKPGASVKVSCKASGYTFTDYYMHWVRQAPGQGLEWMGW | 11 |
| Meso | | INPNSGGTNYAQKFQGRVTMTRDTSISTAYMELSRLRSDDTAVYYCASGW | |
| | | DFDYWGQGTLVTVSS | |
| | VL | DIVMTQSPSSLSASVGDRVTITCRASQSIRYYLSWYQQKPGKAPKLLIYT | 12 |
| | | ASILQNGVPSRFSGSGSGTDFTLTISSLQPEDFATYYCLQTYTTPDFGPG | |
| | | TKVEIK | |
| | scFv | QVQLVQSGAEVEKPGASVKVSCKASGYTFTDYYMHWVRQAPGQGLEWMGW | 13 |
| | | INPNSGGTNYAQKFQGRVTMTRDTSISTAYMELSRLRSDDTAVYYCASGW | |
| | | DFDYWGQGTLVTVSSGSTSGSGKPGSGEGSTKDIVMTQSPSSLSASVGDR | |
| | | VTITCRASQSIRYYLSWYQQKPGKAPKLLIYTASILQNGVPSRFSGSGSG | |
| | | TDFTLTISSLQPEDFATYYCLQTYTTPDFGPGTKVEIK | |
| Anti- | VH | QIQLVQSGPELKKPGETVKISCKASGYTFTDYSINWVKRAPGKGLKWMGW | 14 |
| BCMA | | INTETREPAYAYDFRGRFAFSLETSASTAYLQINNLKYEDTATYFCALDY | |
| | | SYAMDYWGQGTSVTVSS | |
| | VL | DIVLTQSPPSLAMSLGKRATISCRASESVTILGSHLIHWYQQKPGQPPTL | 15 |
| | | LIQLASNVQTGVPARFSGSGSRTDFTLTIDPVEEDDVAVYYCLQSRTIPR | |
| | | TFGGGTKLEIK | |
| | scFv | DIVLTQSPPSLAMSLGKRATISCRASESVTILGSHLIHWYQQKPGQPPTL | 16 |
| | | LIQLASNVQTGVPARFSGSGSRTDFTLTIDPVEEDDVAVYYCLQSRTIPR | |
| | | TFGGGTKLEIKGSTSGSGKPGSGEGSTKGQIQLVQSGPELKKPGETVKIS | |
| | | CKASGYTFTDYSINWVKRAPGKGLKWMGWINTETREPAYAYDFRGRFAFS | |
| | | LETSASTAYLQINNLKYEDTATYFCALDYSYAMDYWGQGTSVTVSS | |
| Anti- | VH | QVQLQQSGPELKKPGETVKISCKASGYPFTNYGMNWVKQAPGQGLKWMGW | 17 |
| HER2 | | INTSTGESTFADDFKGRFDFSLETSANTAYLQINNLKSEDMATYFCARWE | |
| | | VYHGYVPYWGQGTTVTVSS | |
| | VL | DIQLTQSHKFLSTSVGDRVSITCKASQDVYNAVAWYQQKPGQSPKLLIYS | 18 |
| | | ASSRYTGVPSRFTGSGSGPDFTFTISSVQAEDLAVYFCQQHFRTPFTFGS | |
| | | GTKLEIK | |
| | scFv | QVQLQQSGPELKKPGETVKISCKASGYPFTNYGMNWVKQAPGQGLKWMGW | 19 |
| | | INTSTGESTFADDFKGRFDFSLETSANTAYLQINNLKSEDMATYFCARWE | |
| | | VYHGYVPYWGQGTTVTVSSGSTSGSGKPGSGEGSTKDIQLTQSHKFLSTS | |
| | | VGDRVSITCKASQDVYNAVAWYQQKPGQSPKLLIYSASSRYTGVPSRFTG | |
| | | SGSGPDFTFTISSVQAEDLAVYFCQQHFRTPFTFGSGTKLEIK | |
| Anti- | VH | QVQLQQSGAELVRPGASVKLSCKASGYTFTDYEMHWVKQTPVHGLKWIGA | 20 |
| GPC3 | | LDPKTGDTAYSQKFKGKATLIADKSSSTAYMELRSLISEDSAVYYCTRFY | |
| | | SYTYWGQGTLVTVS | |
| | VL | DVVMTQTPLSLPVSLGDQASISCRSSQSLVHSNGNTYLHWYLQKPGQSPK | 21 |
| | | LLIYKVSNRFSGVPDRFSGSGSGTDFTLKISRVEAEDLGVYFCSQNTHVP | |
| | | PTFGSGTKLEIK | |
| | scFv | QVQLQQSGAELVRPGASVKLSCKASGYTFTDYEMHWVKQTPVHGLKWIGA | 22 |
| | | LDPKTGDTAYSQKFKGKAILTADKSSSTAYMELRSLTSEDSAVYYCTRFY | |
| | | SYTYWGQGTLVTVSGSTSGSGKPGSGEGSTKDVVMTQTPLSLPVSLGDQA | |
| | | SISCRSSQSLVHSNGNTYLHWYLQKPGQSPKLLIYKVSNRFSGVPDRFSG | |
| | | SGSGIDFTLKISRVEAEDLGVYFCSQNTHVPPTFGSGTKLEIK | |
| Anti- | VH | QVQLQQPGAELVRPGASVKLSCKASGYTFTSYWINWVKQRPGQGLEWIGN | 23 |
| Claudin | | IYPSDSYTNYNQKFKDKATLTVDKSSSTAYMQLSSPTSEDSAVYYCTRSW | |
| 18.2 | | RGNSFDYWGQGTTLTVSS | |
| | VL | DIVMTQSPSSLTVTAGEKVTMSCKSSQSLLNSGNQKNYLTWYQQKPGQPP | 24 |
| | | KLLIYWASTRESGVPDRFTGSGSGTDFTLTISSVQAEDLAVYYCQNDYSY | |
| | | PFTFGSGTKLEIKR | |
| | scFv | QVQLQQPGAELVRPGASVKLSCKASGYTFTSYWINWVKQRPGQGLEWIGN | 25 |
| | | IYPSDSYTNYNQKFKDKATLTVDKSSSTAYMQLSSPTSEDSAVYYCTRSW | |
| | | RGNSFDYWGQGTTLTVSSGSTSGSGKPGSGEGSTKDIVMTQSPSSLTVTA | |
| | | GEKVTMSCKSSQSLLNSGNQKNYLTWYQQKPGQPPKLLIYWASTRESGVP | |
| | | DRFTGSGSGTDFTLTISSVQAEDLAVYYCQNDYSYPFTFGSGTKLEIKR | |
| Anti- | VH | EVKLQESGPGLVAPSQSLSVTCTVSGVSLPDYGVSWIRQPPRKGLEWLGV | 59 |
| CD19 | | IWGSETTYYNSALKSRLTIIKDNSKSQVFLKMNSLQTDDTAIYYCAKHYY | |
| | | YGGSYAMDYWGQGTSVTVSS | |
| | VL | DIQMTQTTSSLSASLGDRVTISCRASQDISKYLNWYQQKPDGTVKLLIYH | 60 |
| | | TSRLHSGVPSRFSGSGSGTDYSLTISNLEQEDIATYFCQQGNTLPYTFGG | |
| | | GTKLEIT | |
| | scFv | EVKLQESGPGLVAPSQSLSVTCTVSGVSLPDYGVSWIRQPPRKGLEWLGV | 61 |
| | (VH- | IWGSETTYYNSALKSRLTIIKDNSKSQVFLKMNSLQTDDTAIYYCAKHYY | |
| | VL) | YGGSYAMDYWGQGTSVTVSSGSTSGSGKPGSGEGSTKGDIQMTQTTSSLS | |
| | | ASLGDRVTISCRASQDISKYLNWYQQKPDGTVKLLIYHTSRLHSGVPSRF | |
| | | SGSGSGTDYSLTISNLEQEDIATYFCQQGNTLPYTFGGGTKLEIT | |
| | scFv | DIQMTQTTSSLSASLGDRVTISCRASQDISKYLNWYQQKPDGTVKLLIYH | 62 |
| | (VL- | TSRLHSGVPSRFSGSGSGTDYSLTISNLEQEDIATYFCQQGNTLPYTFGG | |
| | VH) | GTKLEITGGGGSGGGGSGGGGSEVKLQESGPGLVAPSQSLSVTCTVSGVS | |
| | | LPDYGVSWIRQPPRKGLEWLGVIWGSETTYYNSALKSRLTIIKDNSKSQV | |
| | | FLKMNSLQTDDTAIYYCAKHYYYGGSYAMDYWGQGTSVTVSS | |

| | | |
|---|---|---|
| Meso/BCMA | MALPVTALLLPLALLLHAARPQVQLVQSGAEVEKPGASVKVSCKASGYTF | 26 |
| bispecific CAR | TDYYMHWVRQAPGQGLEWMGWINPNSGGTNYAQKFQGRVTMTRDTSISTA | (with |
| | YMELSRLRSDDTAVYYCASGWDFDYWGQGTLVTVSSGSTSGSGKPGSGEG | signal |
| | STKDIVMTQSPSSLSASVGDRVTITCRASQSIRYYLSWYQQKPGKAPKLL | peptide) |
| | IYTASILQNGVPSRFSGSGSGTDFTLTISSLQPEDFATYYCLQTYTTPDF | |
| | GPGTKVEIKSGGGSDPGGGGSGGGGSPAGDIVLTQSPPSLAMSLGKRATI | |
| | SCRASESVTILGSHLIHWYQQKPGQPPTLLIQLASNVQTGVPARFSGSGS | 27 |
| | RTDFTLTIDPVEEDDVAVYYCLQSRTIPRTFGGGTKLEIKGSTSGSGKPG | (without |
| | SGEGSTKGQIQLVQSGPELKKPGETVKISCKASGYTFTDYSINWVKRAPG | signal |
| | KGLKWMGWINTETREPAYAYDFRGRFAFSLETSASTAYLQINNLKYEDTA | peptide) |
| | TYFCALDYSYAMDYWGQGTSVTVSSTTTPAPRPPTPAPTIASQPLSLRPE | |
| | ACRPAAGGAVHTRGLDFACDIYIWAPLAGTCGVLLLSLVITLYCKRGRKK | |
| | LLYIFKQPFMRPVQTTQEEDGCSCRFPEEEEGGCELNCRNTGPWLKKVLK | |
| | CNTPDPSKFFSQLSSEHGGDVQKWLSSPFPSSSFSPGGLAPEISPLEVLE | |
| | RDKVTQLLPLNTDAYLSLQELQGQDPTHLVRVKFSRSADAPAYKQGQNQL | |
| | YNELNLGRREEYDVLDKRRGRDPEMGGKPRRKNPQEGLYNELQKDKMAEA | |
| | YSEIGMKGERRRGKGHDGLYQGLSTATKDTYDAYRHQALPPR | |
| HER2/BCMA | MALPVTALLLPLALLLHAARPQVQLQQSGPELKKPGETVKISCKASGYPF | 28 |
| bispecific CAR | TNYGMNWVKQAPGQGLKWMGWINTSTGESTFADDFKGRFDFSLETSANTA | (with |
| | YLQINNLKSEDMATYFCARWEVYHGYVPYWGQGTTVTVSSGSTSGSGKPG | signal |
| | SGEGSTKDIQLTQSHKFLSTSVGDRVSITCKASQDVYNAVAWYQQKPGQS | peptide) |
| | PKLLIYSASSRYTGVPSRFTGSGSGPDFTFTISSVQAEDLAVYFCQQHFR | |
| | TPFTFGSGTKLEIKSGGGSDPGGGGSGGGGSPAGDIVLTQSPPSLAMSLG | |
| | KRATISCRASESVTILGSHLIHWYQQKPGQPPTLLIQLASNVQTGVPARF | 29 |
| | SGSGSRTDFTLTIDPVEEDDVAVYYCLQSRTIPRTFGGGTKLEIKGSTSG | (without |
| | SGKPGSGEGSTKGQIQLVQSGPELKKPGETVKISCKASGYTFTDYSINWV | signal |
| | KRAPGKGLKWMGWINTETREPAYAYDFRGRFAFSLETSASTAYLQINNLK | peptide) |
| | YEDTATYFCALDYSYAMDYWGQGISVTVSSTTTPAPRPPTPAPTIASQPL | |
| | SLRPEACRPAAGGAVHIRGLDFACDIYIWAPLAGTCGVLLLSLVITLYCK | |
| | RGRKKLLYIFKQPFMRPVQTTQEEDGCSCRFPEEEEGGCELNCRNTGPWL | |
| | KKVLKCNTPDPSKFFSQLSSEHGGDVQKWLSSPFPSSSFSPGGLAPEISP | |
| | LEVLERDKVTQLLPLNTDAYLSLQELQGQDPTHLVRVKFSRSADAPAYKQ | |
| | GQNQLYNELNLGRREEYDVLDKRRGRDPEMGGKPRRKNPQEGLYNELQKD | |
| | KMAEAYSEIGMKGERRRGKGHDGLYQGLSTATKDTYDAYRHQALPPR | |
| GPC3/BCMA | MALPVTALLLPLALLLHAARPQVQLQQSGAELVRPGASVKLSCKASGYTF | 30 |
| bispecific CAR | TDYEMHWVKQTPVHGLKWIGALDPKTGDTAYSQKFKGKATLTADKSSSTA | (with |
| | YMELRSLTSEDSAVYYCTRFYSYTYWGQGTLVTVSGSTSGSGKPGSGEGS | signal |
| | TKDVVMTQTPLSLPVSLGDQASISCRSSQSLVHSNGNTYLHWYLQKPGQS | peptide) |
| | PKLLIYKVSNRFSGVPDRFSGSGSGTDFTLKISRVEAEDLGVYFCSQNTH | |
| | VPPTFGSGTKLEIKSGGGSDPGGGGSGGGGSPAGDIVLTQSPPSLAMSLG | |
| | KRATISCRASESVTILGSHLIHWYQQKPGQPPTLLIQLASNVQTGVPARF | 31 |
| | SGSGSRTDFTLTIDPVEEDDVAVYYCLQSRTIPRTFGGGTKLEIKGSTSG | (without |
| | SGKPGSGEGSTKGQIQLVQSGPELKKPGETVKISCKASGYTFTDYSINWV | signal |
| | KRAPGKGLKWMGWINTETREPAYAYDFRGRFAFSLETSASTAYLQINNLK | peptide) |
| | YEDTATYFCALDYSYAMDYWGQGISVTVSSTTTPAPRPPTPAPTIASQPL | |
| | SLRPEACRPAAGGAVHTRGLDFACDIYIWAPLAGTCGVLLLSLVITLYCK | |
| | RGRKKLLYIFKQPFMRPVQTTQEEDGCSCRFPEEEEGGCELNCRNTGPWL | |
| | KKVLKCNTPDPSKFFSQLSSEHGGDVQKWLSSPFPSSSFSPGGLAPEISP | |
| | LEVLERDKVTQLLPLNTDAYLSLQELQGQDPTHLVRVKFSRSADAPAYKQ | |
| | GQNQLYNELNLGRREEYDVLDKRRGRDPEMGGKPRRKNPQEGLYNELQKD | |
| | KMAEAYSEIGMKGERRRGKGHDGLYQGLSTATKDTYDAYRHQALPPR | |
| Claudin | MALPVTALLLPLALLLHAARPQVQLQQPGAELVRPGASVKLSCKASGYTF | 32 |
| 18.2/BCMA | TSYWINWVKQRPGQGLEWIGNIYPSDSYTNYNQKFKDKATLTVDKSSSTA | (with |
| bispecific CAR | YMQLSSPTSEDSAVYYCTRSWRGNSFDYWGQGTTLTVSSGSTSGSGKPGS | signal |
| | GEGSTKDIVMTQSPSSLTVTAGEKVTMSCKSSQSLLNSGNQKNYLTWYQQ | peptide) |
| | KPGQPPKLLIYWASTRESGVPDRFTGSGSGIDFTLTISSVQAEDLAVYYC | |
| | QNDYSYPFTFGSGTKLEIKRSGGGSDPGGGGSGGGGSPAGDIVLTQSPPS | |
| | LAMSLGKRATISCRASESVTILGSHLIHWYQQKPGQPPTLLIQLASNVQT | 33 |
| | GVPARFSGSGSRTDFTLTIDPVEEDDVAVYYCLQSRTIPRTFGGGTKLEI | (without |
| | KGSTSGSGKPGSGEGSTKGQIQLVQSGPELKKPGETVKISCKASGYTFTD | signal |
| | YSINWVKRAPGKGLKWMGWINTETREPAYAYDFRGRFAFSLETSASTAYL | peptide) |
| | QINNLKYEDTATYFCALDYSYAMDYWGQGISVTVSSTTTPAPRPPTPAPT | |
| | IASQPLSLRPEACRPAAGGAVHTRGLDFACDIYIWAPLAGTCGVLLLSLV | |
| | ITLYCKRGRKKLLYIFKQPFMRPVQTTQEEDGCSCRFPEEEEGGCELNCR | |
| | NTGPWLKKVLKCNTPDPSKFFSQLSSEHGGDVQKWLSSPFPSSSFSPGGL | |
| | APEISPLEVLERDKVTQLLPLNTDAYLSLQELQGQDPTHLVRVKFSRSAD | |
| | APAYKQGQNQLYNELNLGRREEYDVLDKRRGRDPEMGGKPRRKNPQEGLY | |
| | NELQKDKMAEAYSEIGMKGERRRGKGHDGLYQGLSTATKDTYDAYRHQAL | |
| | PPR | |
| CD19/BCMA | MALPVTALLLPLALLLHAARPEVKLQESGPGLVAPSQSLSVTCTVSGVSL | 57 |
| bispecific CAR | PDYGVSWIRQPPRKGLEWLGVIWGSETTYYNSALKSRLTIIKDNSKSQVF | (with |
| | LKMNSLQTDDTAIYYCAKHYYYGGSYAMDYWGQGTSVTVSSGSTSGSGKP | signal |
| | GSGEGSTKGDIQMTQTTSSLSASLGDRVTISCRASQDISKYLNWYQQKPD | peptide) |
| | GTVKLLIYHTSRLHSGVPSRFSGSGSGTDYSLTISNLEQEDIATYFCQQG | |
| | NTLPYTFGGGTKLEITSGGGSDPGGGGSGGGGSPAGDIVLTQSPPSLAMS | 58 |
| | LGKRATISCRASESVTILGSHLIHWYQQKPGQPPTLLIQLASNVQTGVPA | (without |
| | RFSGSGSRTDFTLTIDPVEEDDVAVYYCLQSRTIPRTFGGGTKLEIKGST | signal |
| | SGSGKPGSGEGSTKGQIQLVQSGPELKKPGETVKISCKASGYTFTDYSIN | peptide) |
| | WVKRAPGKGLKWMGWINTETREPAYAYDERGRFAFSLETSASTAYLQINN | |
| | LKYEDTATYFCALDYSYAMDYWGQGTSVTVSSTTTPAPRPPTPAPTIASQ | |
| | PLSLRPEACRPAAGGAVHTRGLDFACDIYIWAPLAGTCGVLLLSLVITLY | |
| | CKRGRKKLLYIFKQPFMRPVQTTQEEDGCSCRFPEEEEGGCELNCRNTGP | |
| | WLKKVLKCNTPDPSKFFSQLSSEHGGDVQKWLSSPFPSSSFSPGGLAPEI | |
| | SPLEVLERDKVTQLLPLNTDAYLSLQELQGQDPTHLVRVKFSRSADAPAY | |
| | KQGQNQLYNELNLGRREEYDVLDKRRGRDPEMGGKPRRKNPQEGLYNELQ | |
| | KDKMAEAYSEIGMKGERRRGKGHDGLYQGLSTATKDTYDAYRHQALPPR | |
| Truncated CD27 | MALPVTALLLPLALLLHAARPTPAPKSCPERHYWAQGKLCCQMCEPGTFL | 34 |
| | VKDCDQHRKAAQCDPCIPGVSFSPDHHTRPHCESCRHCNSGLLVRNCTIT | (with |
| | ANAECACRNGWQCRDKECTECDPLPNPSLTARSSQALSPHPQPTHLPYVS | signal |
| | EMLEARTAGHMQTLADFRQLPARTLSTHWPPQRSLCSSDFIRILVIFSGM | peptide) |
| | FLVFTLAGALFLH | 35 |
| | | (without |
| | | signal |
| | | peptide) |
| Anti-CD70 CAR | MALPVTALLLPLALLLHAARPTPAPKSCPERHYWAQGKLCCQMCEPGTFL | 36 |
| | VKDCDQHRKAAQCDPCIPGVSFSPDHHTRPHCESCRHCNSGLLVRNCTIT | (with |
| | ANAECACRNGWQCRDKECTECDPLPNPSLTARSSQALSPHPQPTHLPYVS | signal |
| | EMLEARTAGHMQTLADFRQLPARTLSTHWPPQRSLCSSDFIRILVIFSGM | peptide) |
| | FLVFTLAGALFLHRVKFSRSADAPAYKQGQNQLYNELNLGRREEYDVLDK | |
| | RRGRDPEMGGKPRRKNPQEGLYNELQKDKMAEAYSEIGMKGERRRGKGHD | 37 |
| | GLYQGLSTATKDTYDALHMQALPPR | (without |
| | | signal |
| | | peptide) |
| Anti-BCMA | MALPVTALLLPLALLLHAARPDIVLTQSPPSLAMSLGKRATISCRASESV | 38 |
| CAR | TILGSHLIHWYQQKPGQPPTLLIQLASNVQTGVPARFSGSGSRTDFTLTI | (with |
| | DPVEEDDVAVYYCLQSRTIPRIFGGGTKLEIKGSTSGSGKPGSGEGSTKG | signal |
| | QIQLVQSGPELKKPGETVKISCKASGYTFTDYSINWVKRAPGKGLKWMGW | peptide) |
| | INTETREPAYAYDFRGRFAFSLETSASTAYLQINNLKYEDTATYFCALDY | |
| | SYAMDYWGQGTSVTVSSITTPAPRPPTPAPTIASQPLSLRPEACRPAAGG | 39 |
| | AVHTRGLDFACDIYIWAPLAGTCGVLLLSLVITLYCKRGRKKLLYIFKQP | (without |
| | FMRPVQTTQEEDGCSCRFPEEEEGGCELNCRNTGPWLKKVLKCNTPDPSK | signal |
| | FFSQLSSEHGGDVQKWLSSPFPSSSFSPGGLAPEISPLEVLERDKVTQLL | peptide) |
| | PLNTDAYLSLQELQGQDPTHLVRVKFSRSADAPAYKQGQNQLYNELNLGR | |
| | REEYDVLDKRRGRDPEMGGKPRRKNPQEGLYNELQKDKMAEAYSEIGMKG | |
| | ERRRGKGHDGLYQGLSTATKDTYDAYRHQALPPR | |
| NY-ESO-1 | METLLGVSLVILWLQLARVNSQQGEEDPQALSIQEGENATMNCSYKTSIN | 40 |
| specific TCRα | NLQWYRQNSGRGLVHLILIRSNEREKHSGRLRVTLDTSKKSSSLLITASR | |
| | AADTASYFCATDGAGKSTFGDGTTLTVKPNIQKPDPAVYQLRDSKSSDKS | |
| | VCLFTDFDSQTNVSQSKDSDVYITDKTVLDMRSMDFKSNSAVAWSNKSDF | |
| | ACANAFNNSIIPADTFFPSPESSCDVKLVEKSFETDINLNFQNLSVIGFR | |
| | ILLLKVAGFNLLMTLRLWSS | |
| NY-ESO-1 | MDSWTLCCVSLCILVAKHTDAGVIQSPRHEVTEMGQEVTLRCKPISGHDY | 41 |
| specific TCRß | LFWYRQTMMRGLELLIYFNNNVPIDDSGMPEDRFSAKMPNASFSTLKIQP | |
| | SEPRDSAVYFCASTIGAQPQHFGDGIRLSILEDLNKVFPPEVAVFEPSEA | |
| | EISHTQKATLVCLATGFFPDHVELSWWVNGKEVHSGVSTDPQPLKEQPAL | |
| | NDSRYCLSSRLRVSATFWQNPRNHFRCQVQFYGLSENDEWTQDRAKPVTQ | |
| | IVSAEAWGRADCGFTSVSYQQGVLSATILYEILLGKATLYAVLVSALVLM | |
| | AMVKRKD | |
| Anti-CD33 VHH | EVQLVESGGGLVQPGGSLRLSCAASGFTFSSYGMSWVRQAPRQGLEWVAN | 42 |
| | IKQDGSEKYYADSVKGRFTISRDNSKNTLYLQMNSLRAEDTATYYCAKEN | |
| | VDWGQGTLVTVSS | |
| Linker D | SDPGGGGSEAAAKGSGSGAPEFLGGPGGSDPIEVMYPPPYLDNEKSNGTI | 43 |
| | IHVKGKHLaPSPLFPGPSKP | |
| Linker E | SDPGGGGSESKYGPPGGSGGSDPTTTPAPRPPTPAPTIASQPLSLRPEAa | 44 |
| | RPAAGGAVHTRGLDFAaD | |
| CD3δECD and | FKIPIEELEDRVFVNCNTSITWVEGTVGTLLSDITRLDLGKRILDPRGIY | 45 |
| TM | RCNGTDIYKDKESTVQVHYRMCQSCVELDPATVAGIIVIDVIATLLLALG | |
| | VFCFAG | |
| CD3γECD and | QSIKGNHLVKVYDYQEDGSVLLTCDAEAKNITWFKDGKMIGFLTEDKKKW | 46 |
| TM | NLGSNAKDPRGMYQCKGSQNKSKPLQVYYRMCQNCIELNAATISGFLFAE | |
| | IVSIFVLAVGVYFIAG | |
| Anti-CD123 | EVQLVQSGGGLVQAGGSLRLSCAASGRTVSNYPMAWFRQAPGKEREFVAH | 47 |
| VHH 1 | ISWSGITSILNSVNDRFTISRDNAKNTIYLQMNSLKPEDTAVYYCAAAQR | |
| | PTAGPKGPFGYWGQGTQVTV | |
| Anti-CD123 | QVTLRESGGGLVQAGGSLRLSCKGSGRAINTYAMGWFRQAPGKEREFVAA | 48 |
| VHH 2 | ISWNGGHTRYADSVQGRFAISRDNADNTMYLQMNSLKPEDTAVYHCAAYS | |
| | DYHRIATMEADADSWGQGTQVTV | |
| Anti-CD123 | QVQLVQSGGGSVQAGGSLRLSCAAAGRTQSAVAMGWFRQDPGKDRDFVAA | 49 |
| VHH 3 | IRWSGGNTYYADSAEGRFTISRDNAKNTVYLQMDSLKPEDTAVYSCAISM | |
| | NHFGMYDYWGQGTQVTV | |
| Anti-CD123 | QVQLVQSGGGLVQAGGSLTVSCTASGRAINMYAMGWFRQAPGKEREFVAA | 50 |
| VHH 4 | INWNGAYTQYADSVKGRFTISRDNAKNTMYLQMNSLKPEDTAQYYCSADA | |
| | DYNTYVSPNKRVSYWGQGTQVTV | |
| Anti-CD123 | EVQLVQSGGGLVQAGGSLRLSCRASGRAINSYNMGWFRQAPGKEREFVSA | 51 |
| VHH 5 | INWNGARTYYQDALKGRFAISRDNARNTMYLQMNNLKPEDTAVYYCAAAG | |
| | RWSAAVPSGEDQYNFWGQGTQVTV | |
| Anti-CD123 | EVQLVQSGGGLVQAGGSLRLSCAVSGGTFSSYGMAWFRQPPGKEREWVAS | 52 |
| VHH 6 | NSWIAGSTYYAGSVKGRFTISRDNAKNTVYLQMNSLKPEDTAVYYCAADL | |
| | LATADDEYDYWGQGTQVTV | |
| IL-18 | YFGKLESKLSVIRNLNDQVLFIDQGNRPLFEDMTDSDCRDNAPRTIFIIS | 53 |
| | MYKDSQPRGMAVTISVKCEKISTLSCENKIISFKEMNPPDNIKDTKSDII | |
| | FFQRSVPGHDNKMQFESSSYEGYFLACEKERDLFKLILKKEDELGDRSIM | |
| | FTVQNED | |
| Linker F | GGGGSESKYGPPGGSGGSDP | 54 |
| anti-IFN γscFv | EIVLTQSPGTLSLSPGERAILSCRASQSVSSSYLAWYQQKPGQAPRLLIY | 55 |
| | GASSRATGIPDRFSGSGSGTDFTLTISRLEPEDFAVYYCQRSGGSSFTFG | |
| | PGTKVDIKGGGGSGGGGSGGGGSEVQLVQSGAEVKKPGESLKISCKGSGY | |
| | NFTSYWIGWVRQMPGKGLELMGIIYPGDSDTRYSPSFQGQVTISADKSIS | |
| | TAYLQWSSLKASDTAMYYCGSGSYFYFDLWGRGTLVTVSS | |
| IL-18/anti | YFGKLESKLSVIRNLNDQVLFIDQGNRPLFEDMTDSDCRDNAPRTIFIIS | 56 |
| IFNγ scFv fusion | MYKDSQPRGMAVTISVKCEKISTLSCENKIISFKEMNPPDNIKDTKSDII | |
| polypeptide | FFQRSVPGHDNKMQFESSSYEGYFLACEKERDLFKLILKKEDELGDRSIM | |
| | FTVQNEDRTGGGGSESKYGPPGGSGGSDPEIVLTQSPGILSLSPGERATL | |
| | SCRASQSVSSSYLAWYQQKPGQAPRLLIYGASSRATGIPDRFSGSGSGTD | |
| | FTLTISRLEPEDFAVYYCQRSGGSSFTFGPGTKVDIKGGGGSGGGGSGGG | |
| | GSEVQLVQSGAEVKKPGESLKISCKGSGYNFTSYWIGWVRQMPGKGLELM | |
| | GIIYPGDSDTRYSPSFQGQVTISADKSISTAYLQWSSLKASDTAMYYCGS | |
| | GSYFYFDLWGRGTLVTVSS | |
| Anti-CD19 VHH | QVKLEESGGELVQPGGPLRLSCAASGNIFSINRMGWYRQAPGKQRAFVAS | 68 |
| | ITVRGITNYADSVKGRFTISVDKSKNTIYLQMNALKPEDTAVYYCNAVSS | |
| | NRDPDYWGQGTQVTVSS | |
| Anti-HER2 | EVQLVESGGGLVQAGGSLRLSCAASGITFSINTMGWYRQAPGKQRELVAL | 69 |
| VHH-1 | ISSIGDTYYADSVKGRFTISRDNAKNTVYLQMNSLKPEDTAVYYCKRFRT | |
| | AAQGTDYWGQGTLVTVSS | |
| Anti-HER2- | MALPVTALLLPLALLLHAARPEVQLVESGGGLVQAGGSLRLSCAASGITF | 70 |
| VHH-1-CAR | SINTMGWYRQAPGKQRELVALISSIGDTYYADSVKGRFTISRDNAKNTVY | (with |
| | LQMNSLKPEDTAVYYCKRFRTAAQGTDYWGQGTLVTVSSTTTPAPRPPTP | signal |
| | APTIASQPLSLRPEACRPAAGGAVHTRGLDFACDIYIWAPLAGTCGVLLL | peptide) |
| | SLVITLYCRVKFSRSADAPAYKQGQNQLYNELNLGRREEYDVLDKRRGRD | |
| | PEMGGKPRRKNPQEGLYNELQKDKMAEAYSEIGMKGERRRGKGHDGLYQG | 71 |
| | LSTATKDTYDALHMQALPPR | (without |
| | | signal |
| | | peptide) |
| Anti-HER2 | EVQLVESGGSLVQPGGSLRLSCAASGFTFDDYAMSWVRQVPGKGLEWVSS | 72 |
| VHH-2 | INWSGTHTDYADSVKGRFTISRNNANNTLYLQMNSLKSEDTAVYYCAKNW | |
| | RDAGTTWFEKSGSAGQGTQVTVSS | |
| Anti-HER2- | MALPVTALLLPLALLLHAARPEVQLVESGGSLVQPGGSLRLSCAASGFTF | 73 |
| VHH-2-CAR | DDYAMSWVRQVPGKGLEWVSSINWSGTHTDYADSVKGRFTISRNNANNTL | (with |
| | YLQMNSLKSEDTAVYYCAKNWRDAGTTWFEKSGSAGQGTQVTVSSTTTPA | signal |
| | PRPPTPAPTIASQPLSLRPEACRPAAGGAVHTRGLDFACDIYIWAPLAGT | peptide) |
| | CGVLLLSLVITLYCRVKFSRSADAPAYKQGQNQLYNELNLGRREEYDVLD | |
| | KRRGRDPEMGGKPRRKNPQEGLYNELQKDKMAEAYSEIGMKGERRRGKGH | 74 |
| | DGLYQGLSTATKDTYDALHMQALPPR | (without |
| | | signal |
| | | peptide) |
| Anti- | QLQLVESGGGLVQPGGSLRLSCAASGVDISSDVMAWYRQAPGKQREFVSG | 75 |
| Claudin18.2 | LTRGGSINYADSVKGRFTISRDFAKNTVDLQMNSLKPEDTAVYYCNAEIY | |
| VHH-1 | TGTFYPRSYWGQGTQVTVSS | |
| Anti- | MALPVTALLLPLALLLHAARPQLQLVESGGGLVQPGGSLRLSCAASGVDI | 76 |
| Claudin18.2 | SSDVMAWYRQAPGKQREFVSGLIRGGSINYADSVKGRFTISRDFAKNTVD | (with |
| VHH-1-CAR | LQMNSLKPEDTAVYYCNAEIYTGTFYPRSYWGQGTQVTVSSTTTPAPRPP | signal |
| | TPAPTIASQPLSLRPEACRPAAGGAVHTRGLDFACDIYIWAPLAGTCGVL | peptide) |
| | LLSLVITLYCRVKFSRSADAPAYKQGQNQLYNELNLGRREEYDVLDKRRG | |
| | RDPEMGGKPRRKNPQEGLYNELQKDKMAEAYSEIGMKGERRRGKGHDGLY | 77 |
| | QGLSTATKDTYDALHMQALPPR | (without |
| | | signal |
| | | peptide) |
| Anti- | QLQLVESGGGLVQPGGSLRLSCAASGGIFSIGVMGWYRQAPGKQRELVAT | 78 |
| Claudin18.2 | ITSRGSTNYADSVKGRFTISGDNAKNTVYLQMNNLKPEDTAVYYCYADLI | |
| VHH-2 | RPGDFYGMDYWGKGTLVTVSS | |
| Anti- | MALPVTALLLPLALLLHAARPQLQLVESGGGLVQPGGSLRLSCAASGGIF | 79 |
| Claudin18.2 | SIGVMGWYRQAPGKQRELVATITSRGSTNYADSVKGRFTISGDNAKNTVY | (with |
| VHH-2-CAR | LQMNNLKPEDTAVYYCYADLIRPGDFYGMDYWGKGTLVTVSSTTTPAPRP | signal |
| | PTPAPTIASQPLSLRPEACRPAAGGAVHTRGLDFACDIYIWAPLAGTCGV | peptide) |
| | LLLSLVITLYCRVKFSRSADAPAYKQGQNQLYNELNLGRREEYDVLDKRR | |
| | GRDPEMGGKPRRKNPQEGLYNELQKDKMAEAYSEIGMKGERRRGKGHDGL | 80 |
| | YQGLSTATKDTYDALHMQALPPR | (without |
| | | signal |
| | | peptide) |
| Anti-mesothelin | QVQLVQSGGGLVQAGGSLRLSCAPSGSIFGIRTMDWYRQAPGKERELVAR | 81 |
| VHH-1 | ITMDGRVFHADSVKGRFSGSRDGASNAVYLQMNSLKPDDTAVYYCRYSGL | |
| | TSREDYWGPGTQVTVSS | |
| Anti-mesothelin | MALPVTALLLPLALLLHAARPQVQLVQSGGGLVQAGGSLRLSCAPSGSIF | 82 |
| VHH-1-CAR | GIRTMDWYRQAPGKERELVARITMDGRVFHADSVKGRFSGSRDGASNAVY | (with |
| | LQMNSLKPDDTAVYYCRYSGLISREDYWGPGTQVTVSSTTTPAPRPPTPA | signal |
| | PTIASQPLSLRPEACRPAAGGAVHTRGLDFACDIYIWAPLAGTCGVLLLS | peptide) |
| | LVITLYCRVKFSRSADAPAYKQGQNQLYNELNLGRREEYDVLDKRRGRDP | |
| | EMGGKPRRKNPQEGLYNELQKDKMAEAYSEIGMKGERRRGKGHDGLYQGL | 83 |
| | STATKDTYDALHMQALPPR | (without |
| | | signal |
| | | peptide) |
| Anti-mesothelin | QVQLVQSGGGLVHPGGSLRLSCAASGIDLSLYRMRWYRQAPGKERDLVAL | 84 |
| VHH-2 | ITDDGTSYYEDSVKGRFTITRDNPSNKVFLQMNSLKPEDTAVYYCNAETP | |
| | LSPVNYWGQGTQVTVSS | |
| Anti-mesothelin | MALPVTALLLPLALLLHAARPQVQLVQSGGGLVHPGGSLRLSCAASGIDL | 85 |
| VHH-2-CAR | SLYRMRWYRQAPGKERDLVALITDDGTSYYEDSVKGRFTITRDNPSNKVF | (with |
| | LQMNSLKPEDTAVYYCNAETPLSPVNYWGQGTQVTVSSTTTPAPRPPTPA | signal |
| | PTIASQPLSLRPEACRPAAGGAVHTRGLDFACDIYIWAPLAGTCGVLLLS | peptide) |
| | LVITLYCRVKFSRSADAPAYKQGQNQLYNELNLGRREEYDVLDKRRGRDP | |
| | EMGGKPRRKNPQEGLYNELQKDKMAEAYSEIGMKGERRRGKGHDGLYQGL | 86 |
| | STATKDTYDALHMQALPPR | (without |
| | | signal |
| | | peptide) |
| Anti-PSMA | EVQLVESGGGLVQPGGSLTLSCAASRFMISEYHMHWVRQAPGKGLEWVSD | 87 |
| VHH-1 | INPAGTTDYAESVKGRFTISRDNAKNTLYLQMNSLKPEDTAVYYCDSYGY | |
| | RGQGTQVTVSS | |
| Anti-PSMA | MALPVTALLLPLALLLHAARPEVQLVESGGGLVQPGGSLILSCAASREMI | 88 |
| VHH-1-CAR | SEYHMHWVRQAPGKGLEWVSDINPAGTTDYAESVKGRFTISRDNAKNTLY | (with |
| | LQMNSLKPEDTAVYYCDSYGYRGQGTQVTVSSTTTPAPRPPTPAPTIASQ | signal |
| | PLSLRPEACRPAAGGAVHTRGLDFACDIYIWAPLAGTCGVLLLSLVITLY | peptide) |
| | CRVKFSRSADAPAYKQGQNQLYNELNLGRREEYDVLDKRRGRDPEMGGKP | |
| | RRKNPQEGLYNELQKDKMAEAYSEIGMKGERRRGKGHDGLYQGLSTATKD | 89 |
| | TYDALHMQALPPR | (without |
| | | signal |
| | | peptide) |
| Anti-PSMA | EVQLVESGGGLVQPGGSLRLSCAASREMISEYSMHWVRQAPGKGLEWVST | 90 |
| VHH-2 | INPAGTTDYAESVKGRFTISRDNAKNTLYLQMNSLRAEDTAVYYCDGYGY | |
| | RGQGTLVTVSS | |
| Anti-PSMA | MALPVTALLLPLALLLHAARPEVQLVESGGGLVQPGGSLRLSCAASREMI | 91 |
| VHH-2-CAR | SEYSMHWVRQAPGKGLEWVSTINPAGTTDYAESVKGRFTISRDNAKNTLY | (with |
| | LQMNSLRAEDTAVYYCDGYGYRGQGTLVTVSSTTTPAPRPPTPAPTIASQ | signal |
| | PLSLRPEACRPAAGGAVHTRGLDFACDIYIWAPLAGTCGVLLLSLVITLY | peptide) |
| | CRVKFSRSADAPAYKQGQNQLYNELNLGRREEYDVLDKRRGRDPEMGGKP | 92 |
| | RRKNPQEGLYNELQKDKMAEAYSEIGMKGERRRGKGHDGLYQGLSTATKD | (without |
| | TYDALHMQALPPR | signal |
| | | peptide) |
| Anti-GPC3 | QVQLVQSGGGLVQPGGSLRLSCAASYFDFDSYEMSWVRQAPGKGLEWIGS | 93 |
| VHH | IYHSGSTYYNPSLKSRVTISRDNSKNTLYLQMNTLRAEDTATYYCARVNM | |
| | DRFDYWGQGTLVTVSS | |
| Anti-GPC3 | MALPVTALLLPLALLLHAARPQVQLVQSGGGLVQPGGSLRLSCAASYFDF | 94 |
| VHH-CAR | DSYEMSWVRQAPGKGLEWIGSIYHSGSTYYNPSLKSRVTISRDNSKNTLY | (with |
| | LQMNTLRAEDTATYYCARVNMDRFDYWGQGTLVTVSSTTTPAPRPPTPAP | signal |
| | TIASQPLSLRPEACRPAAGGAVHTRGLDFACDIYIWAPLAGTCGVLLLSL | peptide) |
| | VITLYCRVKFSRSADAPAYKQGQNQLYNELNLGRREEYDVLDKRRGRDPE | |
| | MGGKPRRKNPQEGLYNELQKDKMAEAYSEIGMKGERRRGKGHDGLYQGLS | 95 |
| | TATKDTYDALHMQALPPR | (without |
| | | signal |
| | | peptide) |
| Anti-EGFR | QVKLEESGGGSVQTGGSLRLTCAASGRTSRSYGMGWFRQAPGKEREFVSG | 96 |
| VHH | ISWRGDSTGYADSVKGRFTISRDNAKNTVDLQMNSLKPEDTAIYYCAAAA | |
| | GSAWYGTLYEYDYWGQGTQVTVSS | |
| Anti-EGFR | MALPVTALLLPLALLLHAARPQVKLEESGGGSVQTGGSLRLICAASGRIS | 97 |
| VHH-CAR | RSYGMGWFRQAPGKEREFVSGISWRGDSTGYADSVKGRFTISRDNAKNTV | (with |
| | DLQMNSLKPEDTAIYYCAAAAGSAWYGTLYEYDYWGQGTQVTVSSTTTPA | signal |
| | PRPPTPAPTIASQPLSLRPEACRPAAGGAVHTRGLDFACDIYIWAPLAGT | peptide) |
| | CGVLLLSLVITLYCRVKFSRSADAPAYKQGQNQLYNELNLGRREEYDVLD | |
| | KRRGRDPEMGGKPRRKNPQEGLYNELQKDKMAEAYSEIGMKGERRRGKGH | 98 |
| | DGLYQGLSTATKDTYDALHMQALPPR | (without |
| | | signal |
| | | peptide) |
| Anti- | MALPVTALLLPLALLLHAARPEVQLVESGGGLVQAGGSLRLSCAASGITF | 99 |
| HER2(VHH1)- | SINTMGWYRQAPGKQRELVALISSIGDTYYADSVKGRFTISRDNAKNTVY | (with |
| BCMA Bi- | LQMNSLKPEDTAVYYCKRFRTAAQGTDYWGQGTLVTVSSSGGGSGGSGGG | signal |
| specific CAR | SGGSDPAGDIVLTQSPPSLAMSLGKRATISCRASESVTILGSHLIHWYQQ | peptide) |
| | KPGQPPTLLIQLASNVQTGVPARFSGSGSRIDFTLTIDPVEEDDVAVYYC | |
| | LQSRTIPRTFGGGTKLEIKGSTSGSGKPGSGEGSTKGQIQLVQSGPELKK | 100 |
| | PGETVKISCKASGYTFTDYSINWVKRAPGKGLKWMGWINTETREPAYAYD | (without |
| | FRGRFAFSLETSASTAYLQINNLKYEDTATYFCALDYSYAMDYWGQGTSV | signal |
| | TVSSTTTPAPRPPTPAPTIASQPLSLRPEACRPAAGGAVHTRGLDFACDI | peptide) |
| | YIWAPLAGTCGVLLLSLVITLYCKRGRKKLLYIFKQPFMRPVQTTQEEDG | |
| | CSCRFPEEEEGGCELNCRNTGPWLKKVLKCNTPDPSKFFSQLSSEHGGDV | |
| | QKWLSSPFPSSSFSPGGLAPEISPLEVLERDKVTQLLPLNTDAYLSLQEL | |
| | QGQDPTHLVRVKFSRSADAPAYKQGQNQLYNELNLGRREEYDVLDKRRGR | |
| | DPEMGGKPRRKNPQEGLYNELQKDKMAEAYSEIGMKGERRRGKGHDGLYQ | |
| | GLSTATKDTYDAYRHQALPPR | |
| Anti- | MALPVTALLLPLALLLHAARPEVQLVESGGSLVQPGGSLRLSCAASGFTF | 101 |
| HER2(VHH2)- | DDYAMSWVRQVPGKGLEWVSSINWSGTHTDYADSVKGRFTISRNNANNTL | (with |
| BCMA Bi- | YLQMNSLKSEDTAVYYCAKNWRDAGTTWFEKSGSAGQGTQVTVSSSGGGS | signal |
| specific CAR | GGSGGGSGGSDPAGDIVLTQSPPSLAMSLGKRATISCRASESVTILGSHL | peptide) |
| | IHWYQQKPGQPPTLLIQLASNVQTGVPARFSGSGSRIDFTLTIDPVEEDD | |
| | VAVYYCLQSRTIPRTFGGGTKLEIKGSTSGSGKPGSGEGSTKGQIQLVQS | 102 |
| | GPELKKPGETVKISCKASGYTFTDYSINWVKRAPGKGLKWMGWINTETRE | (without |
| | PAYAYDFRGRFAFSLETSASTAYLQINNLKYEDTATYFCALDYSYAMDYW | signal |
| | GQGTSVTVSSTTTPAPRPPTPAPTIASQPLSLRPEACRPAAGGAVHTRGL | peptide) |
| | DFACDIYIWAPLAGTCGVLLLSLVITLYCKRGRKKLLYIFKQPFMRPVQT | |
| | TQEEDGCSCRFPEEEEGGCELNCRNTGPWLKKVLKCNTPDPSKFFSQLSS | |
| | EHGGDVQKWLSSPFPSSSFSPGGLAPEISPLEVLERDKVTQLLPLNTDAY | |
| | LSLQELQGQDPTHLVRVKFSRSADAPAYKQGQNQLYNELNLGRREEYDVL | |
| | DKRRGRDPEMGGKPRRKNPQEGLYNELQKDKMAEAYSEIGMKGERRRGKG | |
| | HDGLYQGLSTATKDTYDAYRHQALPPR | |
| Anti-Claudin 18.2 | MALPVTALLLPLALLLHAARPQLQLVESGGGLVQPGGSLRLSCAASGVDI | 103 |
| (VHH1)-BCMA | SSDVMAWYRQAPGKQREFVSGLIRGGSINYADSVKGRFTISRDFAKNTVD | (with |
| Bi-specific CAR | LQMNSLKPEDTAVYYCNAEIYTGTFYPRSYWGQGTQVTVSSSGGGSGGSG | signal |
| | GGSGGSDPAGDIVLTQSPPSLAMSLGKRATISCRASESVTILGSHLIHWY | peptide) |
| | QQKPGQPPTLLIQLASNVQTGVPARFSGSGSRTDFTLTIDPVEEDDVAVY | |
| | YCLQSRTIPRTFGGGTKLEIKGSTSGSGKPGSGEGSTKGQIQLVQSGPEL | 104 |
| | KKPGETVKISCKASGYTFTDYSINWVKRAPGKGLKWMGWINTETREPAYA | (without |
| | YDFRGRFAFSLETSASTAYLQINNLKYEDTATYFCALDYSYAMDYWGQGT | signal |
| | SVTVSSTTTPAPRPPTPAPTIASQPLSLRPEACRPAAGGAVHTRGLDFAC | peptide) |
| | DIYIWAPLAGTCGVLLLSLVITLYCKRGRKKLLYIFKQPFMRPVQTTQEE | |
| | DGCSCRFPEEEEGGCELNCRNIGPWLKKVLKCNTPDPSKFFSQLSSEHGG | |
| | DVQKWLSSPFPSSSFSPGGLAPEISPLEVLERDKVTQLLPLNTDAYLSLQ | |
| | ELQGQDPTHLVRVKFSRSADAPAYKQGQNQLYNELNLGRREEYDVLDKRR | |
| | GRDPEMGGKPRRKNPQEGLYNELQKDKMAEAYSEIGMKGERRRGKGHDGL | |
| | YQGLSTATKDTYDAYRHQALPPR | |
| Anti-Claudin 18.2 | MALPVTALLLPLALLLHAARPQLQLVESGGGLVQPGGSLRLSCAASGGIF | 105 |
| (VHH2)-BCMA | SIGVMGWYRQAPGKQRELVATITSRGSTNYADSVKGRFTISGDNAKNTVY | (with |
| Bi-specific CAR | LQMNNLKPEDTAVYYCYADLIRPGDFYGMDYWGKGTLVTVSSSGGGSGGS | signal |
| | GGGSGGSDPAGDIVLTQSPPSLAMSLGKRATISCRASESVTILGSHLIHW | peptide) |
| | YQQKPGQPPTLLIQLASNVQTGVPARFSGSGSRIDFTLTIDPVEEDDVAV | |
| | YYCLQSRTIPRTFGGGTKLEIKGSTSGSGKPGSGEGSTKGQIQLVQSGPE | 106 |
| | LKKPGETVKISCKASGYTFTDYSINWVKRAPGKGLKWMGWINTETREPAY | (without |
| | AYDFRGRFAFSLETSASTAYLQINNLKYEDTATYFCALDYSYAMDYWGQG | signal |
| | TSVTVSSITTPAPRPPTPAPTIASQPLSLRPEACRPAAGGAVHTRGLDFA | peptide) |
| | CDIYIWAPLAGTCGVLLLSLVITLYCKRGRKKLLYIFKQPFMRPVQTTQE | |
| | EDGCSCRFPEEEEGGCELNCRNTGPWLKKVLKCNTPDPSKFFSQLSSEHG | |
| | GDVQKWLSSPFPSSSFSPGGLAPEISPLEVLERDKVTQLLPLNTDAYLSL | |
| | QELQGQDPTHLVRVKFSRSADAPAYKQGQNQLYNELNLGRREEYDVLDKR | |
| | RGRDPEMGGKPRRKNPQEGLYNELQKDKMAEAYSEIGMKGERRRGKGHDG | |
| | LYQGLSTATKDTYDAYRHQALPPR | |
| Anti-Meso | MALPVTALLLPLALLLHAARPQVQLVQSGGGLVQAGGSLRLSCAPSGSIF | 107 |
| (VHH1)-BCMA | GIRTMDWYRQAPGKERELVARITMDGRVFHADSVKGRFSGSRDGASNAVY | (with |
| Bi-specific CAR | LQMNSLKPDDTAVYYCRYSGLISREDYWGPGTQVTVSS | signal |
| | SGGGSGGSGGGSGGSDPAGDIVLTQSPPSLAMSLGKRATISCRASESVTI | peptide) |
| | LGSHLIHWYQQKPGQPPTLLIQLASNVQTGVPARFSGSGSRIDFILTIDP | |
| | VEEDDVAVYYCLQSRTIPRTFGGGTKLEIKGSTSGSGKPGSGEGSTKGQI | 108 |
| | QLVQSGPELKKPGETVKISCKASGYTFTDYSINWVKRAPGKGLKWMGWIN | (without |
| | TETREPAYAYDFRGRFAFSLETSASTAYLQINNLKYEDTATYFCALDYSY | signal |
| | AMDYWGQGTSVTVSSTTTPAPRPPTPAPTIASQPLSLRPEACRPAAGGAV | peptide) |
| | HTRGLDFACDIYIWAPLAGTCGVLLLSLVITLYCKRGRKKLLYIFKQPFM | |
| | RPVQTTQEEDGCSCRFPEEEEGGCELNCRNTGPWLKKVLKCNTPDPSKFF | |
| | SQLSSEHGGDVQKWLSSPFPSSSFSPGGLAPEISPLEVLERDKVTQLLPL | |
| | NTDAYLSLQELQGQDPTHLVRVKFSRSADAPAYKQGQNQLYNELNLGRRE | |
| | EYDVLDKRRGRDPEMGGKPRRKNPQEGLYNELQKDKMAEAYSEIGMKGER | |
| | RRGKGHDGLYQGLSTATKDTYDAYRHQALPPR | |
| Anti-Meso | MALPVTALLLPLALLLHAARPQVQLVQSGGGLVHPGGSLRLSCAASGIDL | 109 |
| (VHH2)-BCMA | SLYRMRWYRQAPGKERDLVALITDDGTSYYEDSVKGRFTITRDNPSNKVF | (with |
| Bi-specific CAR | LQMNSLKPEDTAVYYCNAETPLSPVNYWGQGTQVTVSSSGGGSGGSGGGS | signal |
| | GGSDPAGDIVLTQSPPSLAMSLGKRATISCRASESVTILGSHLIHWYQQK | peptide) |
| | PGQPPTLLIQLASNVQTGVPARFSGSGSRTDFTLTIDPVEEDDVAVYYCL | |
| | QSRTIPRTFGGGTKLEIKGSTSGSGKPGSGEGSTKGQIQLVQSGPELKKP | 110 |
| | GETVKISCKASGYTFTDYSINWVKRAPGKGLKWMGWINTETREPAYAYDF | (without |
| | RGRFAFSLETSASTAYLQINNLKYEDTATYFCALDYSYAMDYWGQGTSVT | signal |
| | VSSTTTPAPRPPTPAPTIASQPLSLRPEACRPAAGGAVHTRGLDFACDIY | peptide) |
| | IWAPLAGTCGVLLLSLVITLYCKRGRKKLLYIFKQPFMRPVQTTQEEDGC | |
| | SCRFPEEEEGGCELNCRNTGPWLKKVLKCNTPDPSKFFSQLSSEHGGDVQ | |
| | KWLSSPFPSSSFSPGGLAPEISPLEVLERDKVTQLLPLNTDAYLSLQELQ | |
| | GQDPTHLVRVKFSRSADAPAYKQGQNQLYNELNLGRREEYDVLDKRRGRD | |
| | PEMGGKPRRKNPQEGLYNELQKDKMAEAYSEIGMKGERRRGKGHDGLYQG | |
| | LSTATKDTYDAYRHQALPPR | |
| Anti-PSMA | MALPVTALLLPLALLLHAARPEVQLVESGGGLVQPGGSLTLSCAASRFMI | 111 |
| (VHH1)-BCMA | SEYHMHWVRQAPGKGLEWVSDINPAGITDYAESVKGRFTISRDNAKNTLY | (with |
| Bi-specific CAR | LQMNSLKPEDTAVYYCDSYGYRGQGTQVTVSSSGGGSGGSGGGSGGSDPA | signal |
| | GDIVLTQSPPSLAMSLGKRATISCRASESVTILGSHLIHWYQQKPGQPPT | peptide) |
| | LLIQLASNVQTGVPARFSGSGSRIDFTLTIDPVEEDDVAVYYCLQSRTIP | |
| | RTFGGGTKLEIKGSTSGSGKPGSGEGSTKGQIQLVQSGPELKKPGETVKI | 112 |
| | SCKASGYTFTDYSINWVKRAPGKGLKWMGWINTETREPAYAYDERGRFAF | (without |
| | SLETSASTAYLQINNLKYEDTATYFCALDYSYAMDYWGQGTSVTVSSTTT | signal |
| | PAPRPPTPAPTIASQPLSLRPEACRPAAGGAVHTRGLDFACDIYIWAPLA | peptide) |
| | GTCGVLLLSLVITLYCKRGRKKLLYIFKQPFMRPVQTTQEEDGCSCRFPE | |
| | EEEGGCELNCRNTGPWLKKVLKCNTPDPSKFFSQLSSEHGGDVQKWLSSP | |
| | FPSSSFSPGGLAPEISPLEVLERDKVTQLLPLNTDAYLSLQELQGQDPTH | |
| | LVRVKFSRSADAPAYKQGQNQLYNELNLGRREEYDVLDKRRGRDPEMGGK | |
| | PRRKNPQEGLYNELQKDKMAEAYSEIGMKGERRRGKGHDGLYQGLSTATK | |
| | DTYDAYRHQALPPR | |
| Anti-PSMA | MALPVTALLLPLALLLHAARPEVQLVESGGGLVQPGGSLRLSCAASREMI | 113 |
| (VHH2)-BCMA | SEYSMHWVRQAPGKGLEWVSTINPAGTTDYAESVKGRFTISRDNAKNTLY | (with |
| Bi-specific CAR | LQMNSLRAEDTAVYYCDGYGYRGQGTLVTVSSSGGGSGGSGGGSGGSDPA | signal |
| | GDIVLTQSPPSLAMSLGKRATISCRASESVTILGSHLIHWYQQKPGQPPT | peptide) |
| | LLIQLASNVQTGVPARFSGSGSRIDFTLTIDPVEEDDVAVYYCLQSRTIP | |
| | RTFGGGTKLEIKGSTSGSGKPGSGEGSTKGQIQLVQSGPELKKPGETVKI | 114 |
| | SCKASGYTFTDYSINWVKRAPGKGLKWMGWINTETREPAYAYDFRGRFAF | (without |
| | SLETSASTAYLQINNLKYEDTATYFCALDYSYAMDYWGQGISVTVSSTTT | signal |
| | PAPRPPTPAPTIASQPLSLRPEACRPAAGGAVHIRGLDFACDIYIWAPLA | peptide) |
| | GTCGVLLLSLVITLYCKRGRKKLLYIFKQPFMRPVQTTQEEDGCSCREPE | |
| | EEEGGCELNCRNTGPWLKKVLKCNTPDPSKFFSQLSSEHGGDVQKWLSSP | |
| | FPSSSFSPGGLAPEISPLEVLERDKVTQLLPLNTDAYLSLQELQGQDPTH | |
| | LVRVKFSRSADAPAYKQGQNQLYNELNLGRREEYDVLDKRRGRDPEMGGK | |
| | PRRKNPQEGLYNELQKDKMAEAYSEIGMKGERRRGKGHDGLYQGLSTATK | |
| | DTYDAYRHQALPPR | |
| Anti-GPC3 | MALPVTALLLPLALLLHAARPQVQLVQSGGGLVQPGGSLRLSCAASYFDF | 115 |
| (VHH)-BCMA | DSYEMSWVRQAPGKGLEWIGSIYHSGSTYYNPSLKSRVTISRDNSKNTLY | (with |
| Bi-specific CAR | LQMNTLRAEDTATYYCARVNMDRFDYWGQGTLVTVSSSGGGSGGSGGGSG | signal |
| | GSDPAGDIVLIQSPPSLAMSLGKRATISCRASESVTILGSHLIHWYQQKP | peptide) |
| | GQPPTLLIQLASNVQTGVPARFSGSGSRTDFTLTIDPVEEDDVAVYYCLQ | |
| | SRTIPRIFGGGTKLEIKGSTSGSGKPGSGEGSTKGQIQLVQSGPELKKPG | 116 |
| | ETVKISCKASGYTFTDYSINWVKRAPGKGLKWMGWINTETREPAYAYDER | (without |
| | GRFAFSLETSASTAYLQINNLKYEDTATYFCALDYSYAMDYWGQGTSVTV | signal |
| | SSTTTPAPRPPTPAPTIASQPLSLRPEACRPAAGGAVHTRGLDFACDIYI | peptide) |
| | WAPLAGTCGVLLLSLVITLYCKRGRKKLLYIFKQPFMRPVQTTQEEDGCS | |
| | CRFPEEEEGGCELNCRNTGPWLKKVLKCNTPDPSKFFSQLSSEHGGDVQK | |
| | WLSSPFPSSSFSPGGLAPEISPLEVLERDKVTQLLPLNTDAYLSLQELQG | |
| | QDPTHLVRVKFSRSADAPAYKQGQNQLYNELNLGRREEYDVLDKRRGRDP | |
| | EMGGKPRRKNPQEGLYNELQKDKMAEAYSEIGMKGERRRGKGHDGLYQGL | |
| | STATKDTYDAYRHQALPPR | |
| Anti-EGFR | MALPVTALLLPLALLLHAARPQVKLEESGGGSVQTGGSLRLICAASGRTS | 117 |
| (VHH)-BCMA | RSYGMGWFRQAPGKEREFVSGISWRGDSTGYADSVKGRFTISRDNAKNTV | (with |
| Bi-specific CAR | DLQMNSLKPEDTAIYYCAAAAGSAWYGTLYEYDYWGQGTQVTVSS | signal |
| | SGGGSGGSGGGSGGSDPAGDIVLTQSPPSLAMSLGKRATISCRASESVTI | peptide) |
| | LGSHLIHWYQQKPGQPPTLLIQLASNVQTGVPARFSGSGSRIDFTLTIDP | |
| | VEEDDVAVYYCLQSRTIPRTFGGGTKLEIKGSTSGSGKPGSGEGSTKGQI | 118 |
| | QLVQSGPELKKPGETVKISCKASGYTFTDYSINWVKRAPGKGLKWMGWIN | (without |
| | TETREPAYAYDERGRFAFSLETSASTAYLQINNLKYEDTATYFCALDYSY | signal |
| | AMDYWGQGTSVTVSSTTTPAPRPPTPAPTIASQPLSLRPEACRPAAGGAV | peptide) |
| | HTRGLDFACDIYIWAPLAGTCGVLLLSLVITLYCKRGRKKLLYIFKQPFM | |
| | RPVQTTQEEDGCSCRFPEEEEGGCELNCRNTGPWLKKVLKCNTPDPSKFF | |
| | SQLSSEHGGDVQKWLSSPFPSSSFSPGGLAPEISPLEVLERDKVTQLLPL | |
| | NTDAYLSLQELQGQDPTHLVRVKFSRSADAPAYKQGQNQLYNELNLGRRE | |
| | EYDVLDKRRGRDPEMGGKPRRKNPQEGLYNELQKDKMAEAYSEIGMKGER | |
| | RRGKGHDGLYQGLSTATKDTYDAYRHQALPPR | |
| IL-10 | SPGQGTQSENSCTHFPGNLPNMLRDLRDAFSRVKIFFQMKDQLDNLLLKE | 119 |
| | SLLEDFKGYLGCQALSEMIQFYLEEVMPQAENQDPDIKAHVNSLGENLKT | |
| | LRLRLRRCHRFLPCENKSKAVEQVKNAFNKLQEKGIYKAMSEFDIFINYI | |
| | EAYMTMKIRN | |
| Membrane-bound | MHSSALLCCLVLLTGVRASPGQGTQSENSCTHFPGNLPNMLRDLRDAFSR | 120 |
| IL-10 | VKTFFQMKDQLDNLLLKESLLEDFKGYLGCQALSEMIQFYLEEVMPQAEN | |
| | QDPDIKAHVNSLGENLKTLRLRLRRCHRFLPCENKSKAVEQVKNAFNKLQ | |
| | EKGIYKAMSEFDIFINYIEAYMIMKIRNGSGGGGSGGGGSGGGGSTTTPA | |
| | PRPPTPAPTIASQPLSLRPEACRPAAGGAVHTRGLDFACDIYIWAPLAGT | |
| | CGVLLLSLVITLYC | |
| Membrane-bound | MALPVTALLLPLALLLHAARPYFGKLESKLSVIRNLNDQVLFIDQGNRPL | 121 |
| IL-18 | FEDMTDSDCRDNAPRTIFIISMYKDSQPRGMAVTISVKCEKISTLSCENK | |
| | IISFKEMNPPDNIKDTKSDIIFFQRSVPGHDNKMQFESSSYEGYFLACEK | |
| | ERDLFKLILKKEDELGDRSIMFTVQNEDGSGGGGSGGGGSGGGGSTTTPA | |
| | PRPPTPAPTIASQPLSLRPEACRPAAGGAVHTRGLDFACDIYIWAPLAGT | |
| | CGVLLLSLVITLYC | |
| Membrane-bound | MRISKPHLRSISIQCYLCLLLNSHFLTEAGIHVFILGCFSAGLPKTEANW | 122 |
| IL-15 | VNVISDLKKIEDLIQSMHIDATLYTESDVHPSCKVTAMKCFLLELQVISL | |
| | ESGDASIHDTVENLIILANNSLSSNGNVTESGCKECEELEEKNIKEFLQS | |
| | FVHIVQMFINTSGSGGGGSGGGGSGGGGSTTTPAPRPPTPAPTIASQPLS | |
| | LRPEACRPAAGGAVHTRGLDFACDIYIWAPLAGTCGVLLLSLVITLYC | |
| Membrane-bound | MLLAMVLTSALLLCSVAGQGCPTLAGILDINFLINKMQEDPASKCHCSAN | 123 |
| IL-9 | VTSCLCLGIPSDNCTRPCFSERLSQMTNTTMQTRYPLIFSRVKKSVEVLK | |
| | NNKCPYFSCEQPCNQTTAGNALTFLKSLLEIFQKEKMRGMRGKIGSGGGG | |
| | SGGGGSGGGGSTTTPAPRPPTPAPTIASQPLSLRPEACRPAAGGAVHTRG | |
| | LDFACDIYIWAPLAGTCGVLLLSLVITLYC | |
| Membrane-bound | MRSSPGNMERIVICLMVIFLGTLVHKSSSQGQDRHMIRMRQLIDIVDQLK | 124 |
| IL-21 | NYVNDLVPEFLPAPEDVEINCEWSAFSCFQKAQLKSANTGNNERIINVSI | |
| | KKLKRKPPSTNAGRRQKHRLTCPSCDSYEKKPPKEFLERFKSLLQKMIHQ | |
| | HLSSRTHGSEDSGSGGGGSGGGGGGGGSTTTPAPRPPTPAPTIASQPLS | |
| | LRPEACRPAAGGAVHTRGLDFACDIYIWAPLAGTCGVLLLSLVITLYC | |
| Anti-BCMA | MALPVTALLLPLALLLHAARPDIVLTQSPPSLAMSLGKRATISCRASESV | 125 |
| CAR with 41BB | TILGSHLIHWYQQKPGQPPTLLIQLASNVQTGVPARFSGSGSRIDFTLTI | (with |
| and CD3z | DPVEEDDVAVYYCLQSRTIPRTFGGGTKLEIKGSTSGSGKPGSGEGSTKG | signal |
| ITAM3 | QIQLVQSGPELKKPGETVKISCKASGYTFTDYSINWVKRAPGKGLKWMGW | peptide) |
| | INTETREPAYAYDERGRFAFSLETSASTAYLQINNLKYEDTATYFCALDY | |
| | SYAMDYWGQGTSVTVSSTTTPAPRPPTPAPTIASQPLSLRPEACRPAAGG | 126 |
| | AVHTRGLDFACDIYIWAPLAGTCGVLLLSLVITLYCKRGRKKLLYIFKQP | (without |
| | FMRPVQTTQEEDGCSCRFPEEEEGGCELGMKGERRRGKGHDGLYQGLSTA | signal |
| | TKDTYDALHMQALPPR | peptide) |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method for treating tumor, comprising administering to a subject in need thereof (a) an effective amount of a population of genetically engineered T cells expressing one or more chimeric antigen receptors (CARs); and (b) an effective amount of antigen presenting cells (APCs);
wherein:
(i) the genetically engineered T cells express a bi-specific CAR comprising a first antigen binding moiety specific to a tumor-associated antigen (TAA) and a second antigen binding moiety specific to CD19 or BCMA; or
(ii) the genetically engineered T cells express a T cell receptor (TCR) specific to a TAA and a CAR comprising an antigen binding moiety specific to CD19 or BCMA; and
wherein the APCs express (i) CD19 and/or BCMA, and optionally (ii) the TAA.

2. The method of claim 1, wherein the genetically engineered T cells further express an antagonist of a cytokine, optionally wherein the cytokine is selected from the group consisting of interleukin-1 (IL-1), interleukin-1 (IL-2), interleukin-6 (IL-6), interleukin-7 (IL-7), interleukin-1 (IL-9), interleukin-10 (IL-10), interleukin-12 (IL-12), interleukin-15 (IL-15), interleukin-18 (IL-18), interleukin-21 (IL-21), interleukin-23 (IL-23), interleukin-24 (IL-24), interleukin-33 (IL-33), interleukin-36 (IL-36), GM-CSF, interferon gamma (IFN□), and Chemokine (C-C motif) ligand 19 (CCL19).

3. The method of claim 2, wherein the antagonist of the cytokine is a fusion polypeptide comprising a binding moiety to the cytokine and an immune activating cytokine, optionally wherein the immune activating cytokine is selected from the group consisting of IL-2, IL-7, IL-9, IL-10, IL-12, IL-15, IL-18, IL-21, IL-23, IL-24, IL-36, IL-33, and CCL19.

4. The method of claim 3, wherein the fusion polypeptide comprises a binding moiety to IFNγ fused to IL-18;
optionally wherein the binding moiety to IFNγ is anti-IFNγ scFv, which preferably comprises the amino acid sequence of SEQ ID NO: 55; and/or optionally wherein the IL-18 comprises the amino acid sequence of SEQ ID NO: 53;
preferably wherein the fusion polypeptide comprises the amino acid sequence of SEQ ID NO: 56.

5. The method of claim 1, wherein the subject is a human patient having a solid tumor or a hematopoietic cancer; optionally wherein the hematopoietic cancer is acute myeloblastic leukemia (AML).

6. The method of claim 1, wherein the genetically engineered T cells express the bi-specific CAR of (i), and wherein:
(a) the bi-specific CAR comprises the first antigen binding moiety specific to the TAA and the second antigen binding moiety specific to the CD19, and the APCs express the CD19 and optionally the TAA; or
(b) the bi-specific CAR comprises the first antigen binding moiety specific to the TAA and the second antigen binding moiety specific to the BCMA, and the APCs express the BCMA and optionally the TAA.

7. The method of claim 6, wherein the bi-specific CAR comprises a fusion polypeptide comprising the first antigen binding moiety and the second antigen binding moiety; optionally wherein the first antigen binding moiety and the second moiety are connected via a peptide linker.

8. The method of claim 7, wherein the first antigen binding moiety, the second antigen binding moiety, or both are in a single-chain variable fragment (scFv) format or in a single domain antibody (VHH) format.

9. The method of claim 7, wherein the bi-specific CAR further comprises an intracellular domain, which comprises one or more signaling domains; and optionally a hinge domain and a transmembrane domain connecting the antigen binding moieties and the intracellular domain.

10. The method of claim 9, wherein the bi-specific CAR comprises the hinge domain, which optionally is of CD8, CD28, CD4, CD3, or an IgG molecule.

11. The method of claim 9, wherein the bi-specific CAR comprises the transmembrane domain, which optionally is of CD3, CD4, CD8, CD27 or CD28.

12. The method of claim 9, wherein the intracellular domain comprises a co-stimulatory signaling domain and a cytoplasmic signaling domain.

13. The method of claim 9, wherein the intracellular domain comprises a signaling domain of CD3, FcR, DAP12, 41BB, OX40, CD28, CD27, ICOS, IL-2R, IL-7R, IL-9R, IL-10R, IL-12R, IL18R, IL-21R, or IL-23R, or a combination thereof;
optionally wherein the intracellular domain comprises a co-stimulatory domain of 4-1BB, an IL2Rb signaling domain, and a CD35 signaling domain; preferably wherein the co-stimulatory domain of 4-1BB comprises the amino acid sequence of SEQ ID NO:8, the IL2Rb signaling domain comprises the amino acid sequence of SEQ ID NO:9, and/or the CD3ζ signaling domain comprises the amino acid sequence of SEQ ID NO: 10.

14. The method of claim 1, wherein the genetically engineered T cells express the bi-specific CAR of (i), and wherein the bi-specific CAR comprises a first fusion polypeptide that comprises the first antigen binding moiety and a second fusion polypeptide that comprises the second antigen binding moiety.

15. The method of claim 14, wherein the first antigen binding moiety, the second antigen binding moiety, or both are in a single-chain variable fragment (scFv) format or in a single domain antibody (VHH) format.

16. The method of claim 14, wherein the first antigen binding moiety is in a single-chain variable fragment (scFv) or in a single domain antibody (VHH) format, and wherein the second antigen binding moiety is an extracellular domain of a ligand that binds the TAA.

17. The method of claim 1, wherein the bi-specific CAR comprises one or more of the following:
(a) an scFv fragment specific to BCMA (anti-BCMA scFv), which comprises a heavy chain variable region (VH) and a light chain variable region (VL), wherein optionally the VH of the anti-BCMA scFv comprises the amino acid sequence of SEQ ID NO: 14 and the VL of the anti-BCMA scFv comprises the amino acid sequence of SEQ ID NO: 15, preferably wherein the anti-BCMA scFv comprises the amino acid sequence of SEQ ID NO: 16;
(b) an scFv fragment specific to CD19 (anti-CD19 scFv), which comprises a heavy chain variable region (VH) and a light chain variable region (VL), wherein optionally the VH of the anti-CD19 scFv comprises the amino acid sequence of SEQ ID NO: 59 and the VL of the anti-CD19 scFv comprises the amino acid sequence of SEQ ID NO: 60, preferably wherein the anti-CD19 scFv comprises the amino acid sequence of SEQ ID NO: 61 or 62;
(c) an scFv fragment specific to Meso (anti-MesoscFv), which comprises a heavy chain variable region (VH) and a light chain variable region (VL), wherein optionally the VH of the anti-MesoscFv comprises the amino acid sequence of SEQ ID NO: 11 and the VL of the anti-MesoscFv comprises the amino acid sequence of SEQ ID NO: 12, preferably wherein the anti-MesoscFv comprises the amino acid sequence of SEQ ID NO: 13;
(d) an scFv fragment specific to HER2 (anti-HER2 scFv), which comprises a heavy chain variable region (VH) and a light chain variable region (VL), wherein optionally the VH of the anti-HER2 scFv comprises the amino acid sequence of SEQ ID NO: 17 and the VL of the anti-HER2 scFv comprises the amino acid sequence of SEQ ID NO: 18, preferably wherein the anti-HER2 scFv comprises the amino acid sequence of SEQ ID NO: 19;
(e) an scFv fragment specific to GPC3 (anti-GPC3 scFv), which comprises a heavy chain variable region (VH) and a light chain variable region (VL), wherein optionally the VH of the anti-GPC3 scFv comprises the amino acid sequence of SEQ ID NO: 20 and the VL of the anti-GPC3 scFv comprises the amino acid sequence of SEQ ID NO: 21, preferably wherein the anti-BCMA scFv comprises the amino acid sequence of SEQ ID NO: 22;
(f) an scFv fragment specific to Claudin 18.2 (anti-Claudin 18.2 scFv), which comprises a heavy chain variable region (VH) and a light chain variable region (VL), wherein optionally the VH of the anti-Claudin 18.2 scFv comprises the amino acid sequence of SEQ ID NO: 23 and the VL of the anti-Claudin 18.2 scFv comprises the amino acid sequence of SEQ ID NO: 24, preferably wherein the anti-Claudin 18.2 scFv comprises the amino acid sequence of SEQ ID NO: 25;
(g) anti-CD33 VHH comprising the amino acid sequence of SEQ ID NO:42; and
(h) anti-CD123 VHH comprising the amino acid sequence of any one of SEQ ID NOS 47-52;
(i) anti-HER2 VHH comprising the amino acid sequence of SEQ ID NO: 69 or 72;
(j) anti-Claudin 18.2 VHH comprising the amino acid sequence of SEQ ID NO: 75 or 78;
(k) anti-mesothelin VHH comprising the amino acid sequence of SEQ ID NO: 81 or 84;
(l) anti-PSMAVHH comprising the amino acid sequence of SEQ ID NO: 87 or 90;
(m) anti-GPC3 VHH comprising the amino acid sequence of SEQ ID NO: 93; and
(n) anti-EGFR VHH comprising the amino acid sequence of SEQ ID NO: 96.

18. The method of claim 16, wherein the first antigen binding moiety is an extracellular domain of CD27, which binds CD70, and wherein the second antigen binding moiety is specific to BCMA;
optionally wherein the extracellular domain of CD27 comprises the amino acid sequence of SEQ ID NO: 34 or 35; and/or
optionally wherein the antigen binding moiety specific to BCMA comprises an scFv fragment specific to BCMA (anti-BCMA scFv), which comprises a heavy chain variable region (VH) and a light chain variable region (VL), wherein optionally the VH of the anti-BCMA scFv comprises the amino acid sequence of SEQ ID NO: 14 and the VL of the anti-BCMA scFv comprises the amino acid sequence of SEQ ID NO: 15, preferably wherein the anti-BCMA scFv comprises the amino acid sequence of SEQ ID NO: 16.

19. The method of claim 14, wherein each of the first fusion polypeptide and the second fusion polypeptide comprises an intracellular domain, which comprises one or more signaling domains; and optionally a hinge domain and a transmembrane domain connecting the antigen binding moieties and the intracellular domain.

20. The method of claim 19, wherein each of the first fusion polypeptide and the second fusion polypeptide comprises the hinge domain, which optionally is of CD8, CD28, CD4, CD3, or an IgG molecule.

21. The method of claim 19, wherein each of the first fusion polypeptide and the second fusion polypeptide comprises the transmembrane domain, which optionally is of CD3, CD4, CD8, CD27 or CD28.

22. The method of claim 19, wherein the intracellular domain comprises a co-stimulatory signaling domain and a cytoplasmic signaling domain.

23. The method of claim 19, wherein the intracellular domain comprises a signaling domain of CD3, FcR, DAP12, 41BB, OX40, CD28, CD27, ICOS, IL-2R, IL-7R, IL-9R, IL-10R, IL-12R, IL18R, IL-21R or IL-23R, or a combination thereof;
optionally wherein the intracellular domain comprises a co-stimulatory domain of 4-1BB, an IL2Rb signaling domain, and a CD3ζ signaling domain; preferably wherein the co-stimulatory domain of 4-1BB comprises the amino acid sequence of SEQ ID NO: 8, the IL2Rb signaling domain comprises the amino acid sequence of SEQ ID NO:9, and/or the CD3ζ signaling domain comprises the amino acid sequence of SEQ ID NO: 10.

24. The method of claim 1, wherein the population of genetically engineered T cells express a bi-specific CAR that binds:
(a) Meso and BCMA, wherein the bi-specific CAR optionally comprises the amino acid sequence of SEQ ID NO: 26 or 27, the amino acid sequence of SEQ ID NO: 107 or 108, or the amino acid sequence of SEQ ID NO: 109 or 110;
(b) HER2 and BCMA, wherein the bi-specific CAR optionally comprises the amino acid sequence of SEQ ID NO: 28 or 29, the amino acid sequence of SEQ ID NO: 99 or 100, or the amino acid sequence of SEQ ID NO: 101 or 102;
(c) GPC3 and BCMA, wherein the bi-specific CAR optionally comprises the amino acid sequence of SEQ ID NO: 30 or 31, or the amino acid sequence of SEQ ID NO: 115 or 116;
(d) Claudin 18.2 and BCMA, wherein the bi-specific CAR optionally comprises the amino acid sequence of SEQ ID NO: 32 or 33, the amino acid sequence of SEQ ID NO: 103 or 104, or the amino acid sequence of SEQ ID NO: 105 or 106;
(e) CD19 and BCMA, wherein the bi-specific CAR optionally comprises the amino acid sequence of SEQ ID NO: 57 or 58;
(f) PSMA and BCMA, wherein the bi-specific CAR optionally comprises the amino acid sequence of SEQ ID NO: 111 or 112, or the amino acid sequence of SEQ ID NO: 113 or 114;
(g) EGFR and BCMA, wherein the bi-specific CAR optionally comprises the amino acid sequence of SEQ ID NO: 117 or 118;
(h) CD70 and BCMA, wherein the bi-specific CAR optionally comprises a first CAR polypeptide specific to CD70 and a second CAR polypeptide specific to BCMA, preferably wherein the first CAR polypeptide comprises the amino acid sequence of SEQ ID NO: 36 or 37 and/or the second CAR polypeptide comprises the amino acid sequence of SEQ ID NO: 38 or 39, or the amino acid sequence of SEQ ID NO: 125 or 126;
(i) HER2 and BCMA, wherein the bi-specific CAR optionally comprises a first CAR polypeptide specific to HER2 and a second CAR polypeptide specific to BCMA; preferably wherein the CAR polypeptide specific to HER2 comprises the amino acid sequence of SEQ ID NO: 70 or 71, or the amino acid sequence of SEQ ID NO: 73 or 74, and the CAR polypeptide specific to BCMA comprises the amino acid sequence of SEQ ID NO: 38 or 39, or the amino acid sequence of SEQ ID NO: 125 or 126;
(j) Claudin 18.2 and BCMA, wherein the bi-specific CAR optionally comprises a first CAR polypeptide specific to Claudin 18.2 and a second CAR polypeptide specific to BCMA; preferably wherein the CAR polypeptide specific to Claudin 18.2 comprises the amino acid sequence of SEQ ID NO: 76 or 77, or the amino acid sequence of SEQ ID NO: 79 or 80; and the CAR polypeptide specific to BCMA comprises the amino acid sequence of SEQ ID NO: 38 or 39, or the amino acid sequence of SEQ ID NO: 125 or 126;
(k) mesothelin and BCMA, wherein the bi-specific CAR optionally comprises a first CAR polypeptide specific to mesothelin and a second CAR polypeptide specific to BCMA; preferably wherein the CAR polypeptide specific to mesothelin comprises the amino acid sequence of SEQ ID NO: 82 or 83, or the amino acid sequence of SEQ ID NO: 85 or 86; and the CAR polypeptide specific to BCMA comprises the amino acid sequence of SEQ ID NO: 38 or 39, or the amino acid sequence of SEQ ID NO: 125 or 126;
(l) PSMA and BCMA, wherein the bi-specific CAR optionally comprises a first CAR polypeptide specific to PSMA and a second CAR polypeptide specific to BCMA; preferably wherein the CAR polypeptide specific to PSMA comprises the amino acid sequence of SEQ ID NO: 88 or 89, or the amino acid sequence of SEQ ID NO: 91 or 92; and the CAR polypeptide specific to BCMA comprises the amino acid sequence of SEQ ID NO: 38 or 39, or the amino acid sequence of SEQ ID NO: 125 or 126;
(m) GPC3 and BCMA, wherein the bi-specific CAR optionally comprises a first CAR polypeptide specific to GPC3 and a second CAR polypeptide specific to BCMA; preferably wherein the CAR polypeptide specific to GPC3 comprises the amino acid sequence of SEQ ID NO: 94 or 95, and the CAR polypeptide specific to BCMA comprises the amino acid sequence of SEQ ID NO: 38 or 39, or the amino acid sequence of SEQ ID NO: 125 or 126; or
(n) EGFR and BCMA, wherein the bi-specific CAR optionally comprises a first CAR polypeptide specific to EGFR and a second CAR polypeptide specific to BCMA; preferably wherein the CAR polypeptide specific to EGFR comprises the amino acid sequence of SEQ ID NO: 97 or 98, and the CAR polypeptide specific to BCMA comprises the amino acid sequence of SEQ ID NO: 38 or 39, or the amino acid sequence of SEQ ID NO: 125 or 126.

25. The method of claim 1, wherein the population of genetically engineered T cells expresses the T cell receptor (TCR) specific to the TAA, and wherein the CAR comprises an antigen binding moiety specific to CD19 or BCMA.

26. The method of claim 25, wherein the CAR comprises the antigen binding moiety specific to CD19 and the APCs express CD19 and optionally the TAA; or wherein the CAR comprises the antigen binding moiety specific to BCMA and the APCs express BCMA and optionally TAA.

27. The method of claim 26, wherein the antigen binding moiety is in a single-chain variable fragment (scFv) format or in a single domain antibody (VHH) format.

28. The method of claim 25, wherein the CAR further comprises an intracellular domain, which comprises one or more signaling domains; and optionally a hinge domain and a transmembrane domain connecting the antigen binding moieties and the intracellular domain.

29. The method of claim 28, wherein the CAR comprises the hinge domain, which optionally is of CD8, CD28, CD4, CD3, or an IgG molecule.

30. The method of claim 28, wherein the CAR comprises the transmembrane domain, which optionally is of CD3, CD4, CD8, CD27 or CD28.

31. The method of claim 28, wherein the intracellular domain comprises a co-stimulatory signaling domain and a cytoplasmic signaling domain.

32. The method of claim 28, wherein the intracellular domain comprises a signaling domain of CD3, FcR, DAP12, 41BB, OX40, CD28, CD27, ICOS, IL-2R, IL-7R, IL-9R, IL-10R, IL-12R, IL18R, IL-21R, or IL-23R, or a combination thereof; optionally wherein the intracellular domain comprises a co-stimulatory domain of 4-1BB, an IL2Rb signaling domain, and a CD32 signaling domain; preferably wherein the co-stimulatory domain of 4-1BB comprises the amino acid sequence of SEQ ID NO: 8, the IL2Rb signaling domain comprises the amino acid sequence of SEQ ID NO:9, and/or the CD3ζ signaling domain comprises the amino acid sequence of SEQ ID NO: 10.

33. The method of claim 25, wherein the T cell receptor (TCR) is specific to NY-ESO-1, optionally wherein the TCR comprises a TCRβ chain comprising the amino acid sequence of SEQ ID NO: 40 and a TCRβ chain comprising the amino acid sequence of SEQ ID NO: 41.

34. The method of claim 25, wherein the T cell receptor (TCR) comprises a modified CD3δ chain and a modified CD3γ chain, which collectively comprises a first antigen binding moiety specific to CD33 (anti-CD33 moiety) and a second antigen binding moiety specific to CD123 (anti-CD123 moiety);
optionally wherein the modified CD3δ chain comprises an extracellular and transmembrane domain of CD3δ fused to the anti-CD33 moiety, and the modified CD3γ chain comprises an extracellular and transmembrane domain of CD3γ fused to the anti-CD123 moiety, or vice versa.

35. The method of claim 34, wherein:
(a) the modified CD3δ chain comprises the amino acid sequence of SEQ ID NO: 45;
(b) the modified CD3γ chain comprises the amino acid sequence of SEQ ID NO: 46;
(c) the anti-CD33 moiety is an anti-CD33 VHH, which optionally comprises the amino acid sequence of SEQ ID NO: 42; and/or
(d) the anti-CD123 moiety is an anti-CD123VHH, which optionally comprises the amino acid sequence of any one of SEQ ID NOs: 47-52.

36. The method of claim 25, wherein the antigen binding moiety specific to CD19 in the CAR comprises a VH comprising SEQ ID NO: 59 and a VL comprising SEQ ID NO: 60; optionally wherein the antigen binding moiety specific to CD19 in the CAR is an scFv fragment comprising the amino acid sequence of SEQ ID NO: 61 or 62.

37. The method of claim 25, wherein the antigen binding moiety specific to BCMA in the CAR comprises a VH comprising SEQ ID NO: 14 and a VL comprising SEQ ID NO: 15; optionally wherein the antigen binding moiety specific to BCMA in the CAR is an scFv fragment comprising the amino acid sequence of SEQ ID NO: 16; preferably wherein the CAR is an anti-BCMA CAR comprising the amino acid sequence of SEQ ID NO: 38 or 39, or the amino acid sequence of SEQ ID NO: 125 or 126.

38. The method of claim 25, wherein the TCR is a complex comprising a first fusion polypeptide that comprises an antigen binding moiety to CD33, and a second fusion polypeptide that comprises an antigen binding moiety to CD123, wherein one of the first fusion polypeptide further comprises a transmembrane fragment of CD3δ and the other one further comprises a transmembrane fragment of CD3γ; and optionally wherein the transmembrane fragments of CD3δ and CD3γ are free of intracellular domains of the CD3δ and CD3γ.

39. The method of claim 25, wherein the population of genetically engineered T cells comprise tumor infiltrating T cells (TILs).

40. The method of claim 1, wherein the population of genetically engineered T cells are autologous to the subject.

41. The method of claim 1, wherein the population of genetically engineered T cells are allogeneic to the subject.

42. The method of claim 1, wherein the APC cells comprise immune cells, stem cells, or tumor cells, optionally wherein the immune cells are T-cells, Natural Killer (NK) cells, tumor infiltrating lymphocytes, dendritic cells, macrophages, B cells, neutrophils, eosinophils, basophils, mast cells, myeloid-derived suppressor cells, and/or mesenchymal stem cells; optionally wherein the stem cells are mesenchymal stem cells, and/or optionally wherein the tumor cells are K562 cells.

43. The method of claim 42, wherein the APCs are genetically engineered to express the CD19 and/or the BCMA, and optionally the TAA.

44. The method of claim 42, wherein the APCs are derived from peripheral blood cells, cord blood cells, induced pluripotent stem cells (iPSCs), or an immune cell line.

45. The method of claim 42, wherein the APCs are genetically engineered to further express a membrane bound stimulatory cytokine.

46. The method of claim 45, wherein the membrane bound stimulatory cytokine is IL-10, IL-18, IL-15, IL-9, or IL-21.

47. The method of claim 1, wherein the APCs are autologous to the subject.

48. The method of claim 1, wherein the APCs are allogeneic to the subject.

49. A kit for treating cancer, comprising: (a) the population of genetically engineered T cells set forth in claim 1, and (b) the APCs set forth in claim 1.

50. A population of genetically engineered T cells, comprising genetically engineered T cells expressing:
(i) a bi-specific CAR comprising a first antigen binding moiety specific to a tumor-associated antigen (TAA) and a second antigen binding moiety specific to CD19 or BCMA; or
(ii) a T cell receptor (TCR) specific to a TAA and the CAR comprises an antigen binding moiety specific to CD19 or BCMA.

51. The population of genetically engineered T cells of claim 50, wherein genetically engineered T cells express the bi-specific CAR of (i),
wherein (a) the bi-specific CAR comprises the first antigen binding moiety specific to the TAA and the second antigen binding moiety specific to the CD19, or (b) the bi-specific CAR comprises the first antigen binding moiety specific to the TAA and the second antigen binding moiety specific to the BCMA.

52. The population of genetically engineered T cells of claim 50, wherein genetically engineered T cells express the TCR of (ii), and wherein the TCR is specific to the TAA.

53. A population of genetically engineered antigen-presenting cells (APCs), wherein the APCs express CD19 and/or BCMA, and wherein the APCs are further genetically engineered to express a membrane bound stimulatory cytokine.

54. The population of genetically engineered APCs of claim 53, wherein the APC cells comprise immune cells, stem cells, or tumor cells, optionally wherein the immune cells are T-cells, Natural Killer (NK) cells, tumor infiltrating lymphocytes, dendritic cells, macrophages, B cells, neutrophils, eosinophils, basophils, mast cells, myeloid-derived suppressor cells, and/or mesenchymal stem cells; optionally wherein the stem cells are mesenchymal stem cells, and/or optionally wherein the tumor cells are K562 cells.


SEQUENCE LISTING (126 sequences)

SEQ ID NO: 1 - protein, 558 aa, synthetic construct
  REGION 1..558: note = synthetic
MPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRFFKVTPPPGSGPQNQYGNVLSLPTPTSGLGRAQRWPRTGLGGTAPSYGNPSSDVQADGALGSRSPPGVGPEEEEGEGYEEPDSEEDSEFYENDSNLGQDQLSQDGSGYENPEDEPLGPEDEDSFSNAESYENEDEELTQPVARTMDFLSPHGSAWDPSREATSLAGSQSYEDMRGILYPRTPQLRSIRGQPGPNHEEDADSYENMDNPDGPDPAWGGGGRMGTWSTR

SEQ ID NO: 2 - protein, 21 aa, synthetic construct
  REGION 1..21: note = synthetic
GSGEGRGSLLTCGDVEENPGP

SEQ ID NO: 3 - protein, 22 aa, synthetic construct
  REGION 1..22: note = synthetic
GSGATNFSLLKQAGDVEENPGP

SEQ ID NO: 4 - protein, 185 aa, synthetic construct
  REGION 1..185: note = synthetic
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPPRTLSATEIEKSISAR

SEQ ID NO: 5 - protein, 22 aa, synthetic construct
  REGION 1..22: note = synthetic
MALPVTALLLPLALLLHPRTRP

SEQ ID NO: 6 - protein, 20 aa, synthetic construct
  REGION 1..20: note = synthetic
SGGGSDPGGGGSGGGGSPAG

SEQ ID NO: 7 - protein, 70 aa, synthetic construct
  REGION 1..70: note = synthetic
TTTPAPRPPTPAPTIASQPLSLRPEACRPPRTGGAVHTRGLDFACDIYIWAPLAGTCGVLLLSLVITLYC

SEQ ID NO: 8 - protein, 43 aa, synthetic construct
  REGION 1..43: note = synthetic
KRGRKKLLYIFKQPFMRPVQTTQEEDGCSCRFPEEEEGGCELC

SEQ ID NO: 9 - protein, 94 aa, synthetic construct
  REGION 1..94: note = synthetic
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 10 - protein, 112 aa, synthetic construct
  REGION 1..112: note = synthetic
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 11 - protein, 115 aa, synthetic construct
  REGION 1..115: note = synthetic
QVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSS

SEQ ID NO: 12 - protein, 106 aa, synthetic construct
  REGION 1..106: note = synthetic
DIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIK

SEQ ID NO: 13 - protein, 238 aa, synthetic construct
  REGION 1..238: note = synthetic
QVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGSTSGSGKPGSGEGSTKDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIK

SEQ ID NO: 14 - protein, 117 aa, synthetic construct
  REGION 1..117: note = synthetic
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 15 - protein, 111 aa, synthetic construct
  REGION 1..111: note = synthetic
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 16 - protein, 246 aa, synthetic construct
  REGION 1..246: note = synthetic
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 17 - protein, 119 aa, synthetic construct
  REGION 1..119: note = synthetic
QVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSS

SEQ ID NO: 18 - protein, 107 aa, synthetic construct
  REGION 1..107: note = synthetic
DIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIK

SEQ ID NO: 19 - protein, 243 aa, synthetic construct
  REGION 1..243: note = synthetic
QVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGSTSGSGKPGSGEGSTKDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIK

SEQ ID NO: 20 - protein, 114 aa, synthetic construct
  REGION 1..114: note = synthetic
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVS

SEQ ID NO: 21 - protein, 112 aa, synthetic construct
  REGION 1..112: note = synthetic
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIK

SEQ ID NO: 22 - protein, 243 aa, synthetic construct
  REGION 1..243: note = synthetic
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSGSTSGSGKPGSGEGSTKDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIK

SEQ ID NO: 23 - protein, 118 aa, synthetic construct
  REGION 1..118: note = synthetic
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSS

SEQ ID NO: 24 - protein, 114 aa, synthetic construct
  REGION 1..114: note = synthetic
DIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKR

SEQ ID NO: 25 - protein, 249 aa, synthetic construct
  REGION 1..249: note = synthetic
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSGSTSGSGKPGSGEGSTKDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKR

SEQ ID NO: 26 - protein, 844 aa, synthetic construct
  REGION 1..844: note = synthetic
MALPVTALLLPLALLLHPRTRPQVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGSTSGSGKPGSGEGSTKDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIKSGGGSDPGGGGSGGGGSPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 27 - protein, 822 aa, synthetic construct
  REGION 1..822: note = synthetic
QVQLVQSGAEVEKPGASVKVSCKASGYTFTDYYMHWVRQAPGQGLEWMGWINPNSGGTNYAQKFQGRVTMTRDTSISTAYMELSRLRSDDTAVYYCASGWDFDYWGQGTLVTVSSGSTSGSGKPGSGEGSTKDIVMTQSPSSLSASVGDRVTITCRASQSIRYYLSWYQQKPGKAPKLLIYTASILQNGVPSRFSGSGSGTDFTLTISSLQPEDFATYYCLQTYTTPDFGPGTKVEIKSGGGSDPGGGGSGGGGSPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 28 - protein, 849 aa, synthetic construct
  REGION 1..849: note = synthetic
MALPVTALLLPLALLLHPRTRPQVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGSTSGSGKPGSGEGSTKDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKSGGGSDPGGGGSGGGGSPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 29 - protein, 827 aa, synthetic construct
  REGION 1..827: note = synthetic
QVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGSTSGSGKPGSGEGSTKDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKSGGGSDPGGGGSGGGGSPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 30 - protein, 849 aa, synthetic construct
  REGION 1..849: note = synthetic
MALPVTALLLPLALLLHPRTRPQVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSGSTSGSGKPGSGEGSTKDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKSGGGSDPGGGGSGGGGSPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 31 - protein, 827 aa, synthetic construct
  REGION 1..827: note = synthetic
QVQLQQSGAELVRPGASVKLSCKASGYTFTDYEMHWVKQTPVHGLKWIGALDPKTGDTAYSQKFKGKATLTADKSSSTAYMELRSLTSEDSAVYYCTRFYSYTYWGQGTLVTVSGSTSGSGKPGSGEGSTKDVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQNTHVPPTFGSGTKLEIKSGGGSDPGGGGSGGGGSPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 32 - protein, 855 aa, synthetic construct
  REGION 1..855: note = synthetic
MALPVTALLLPLALLLHPRTRPQVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSGSTSGSGKPGSGEGSTKDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKRSGGGSDPGGGGSGGGGSPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 33 - protein, 833 aa, synthetic construct
  REGION 1..833: note = synthetic
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSGSTSGSGKPGSGEGSTKDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKRSGGGSDPGGGGSGGGGSPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 34 - protein, 327 aa, synthetic construct
  REGION 1..327: note = synthetic
MALPVTALLLPLALLLHPRTRPTPAPKSCPERHYWAQGKLCCQMCEPGTFLVKDCDQHRKPRTQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 305 aa, synthetic construct
  REGION 1..305: note = synthetic
TPAPKSCPERHYWAQGKLCCQMCEPGTFLVKDCDQHRKPRTQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 327 aa, synthetic construct
  REGION 1..327: note = synthetic
MALPVTALLLPLALLLHPRTRPTPAPKSCPERHYWAQGKLCCQMCEPGTFLVKDCDQHRKPRTQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 37 - protein, 305 aa, synthetic construct
  REGION 1..305: note = synthetic
TPAPKSCPERHYWAQGKLCCQMCEPGTFLVKDCDQHRKPRTQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 38 - protein, 586 aa, synthetic construct
  REGION 1..586: note = synthetic
MALPVTALLLPLALLLHPRTRPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 39 - protein, 564 aa, synthetic construct
  REGION 1..564: note = synthetic
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 40 - protein, 271 aa, synthetic construct
  REGION 1..271: note = synthetic
METLLGVSLVILWLQLARVNSQQGEEDPQALSIQEGENATMNCSYKTSINNLQWYRQNSGRGLVHLILIRSNEREKHSGRLRVTLDTSKKSSSLLITASRPRTDTASYFCATDGAGKSTFGDGTTLTVKPNIQKPDPAVYQLRDSKSSDKSVCLFTDFDSQTNVSQSKDSDVYITDKTVLDMRSMDFKSNSAVAWSNKSDFACANAFNNSIIPADTFFPSPESSCDVKLVEKSFETDTNLNFQNLSVIGFRILLLKVAGFNLLMTLRLWSS

SEQ ID NO: 41 - protein, 307 aa, synthetic construct
  REGION 1..307: note = synthetic
MDSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASTIGAQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKD

SEQ ID NO: 42 - protein, 114 aa, synthetic construct
  REGION 1..114: note = synthetic
EVQLVESGGGLVQPGGSLRLSCPRTSGFTFSSYGMSWVRQAPRQGLEWVANIKQDGSEKYYADSVKGRFTISRDNSKNTLYLQMNSLRAEDTATYYCAKENVDWGQGTLVTVSS

SEQ ID NO: 43 - protein, 71 aa, synthetic construct
  REGION 1..71: note = synthetic
SDPGGGGSEPRTAKGSGSGAPEFLGGPGGSDPIEVMYPPPYLDNEKSNGTIIHVKGKHLAPSPLFPGPSKP

SEQ ID NO: 44 - protein, 71 aa, synthetic construct
  REGION 1..71: note = synthetic
SDPGGGGSESKYGPPGGSGGSDPTTTPAPRPPTPAPTIASQPLSLRPEPRTRPPRTGGAVHTRGLDFPRTD

SEQ ID NO: 45 - protein, 106 aa, synthetic construct
  REGION 1..106: note = synthetic
FKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAG

SEQ ID NO: 46 - protein, 117 aa, synthetic construct
  REGION 1..117: note = synthetic
QSIKGNHLVKVYDYQEDGSVLLTCDAEAKNITWFKDGKMIGFLTEDKKKWNLGSNAKDPRGMYQCKGSQNKSKPLQVYYRMCQNCIELNPRTTISGFLFAEIVSIFVLAVGVYFIAG

SEQ ID NO: 47 - protein, 120 aa, synthetic construct
  REGION 1..120: note = synthetic
EVQLVQSGGGLVQAGGSLRLSCAVSGGTFSSYGMAWFRQPPGKEREWVASNSWIAGSTYYAGSVKGRFTISRDNAKNTVYLQMNSLKPEDTAVYYCPRTDLLATADDEYDYWGQGTQVTV

SEQ ID NO: 48 - protein, 119 aa, synthetic construct
  REGION 1..119: note = synthetic
QVQLVQSGGGSVQAGGSLRLSCPRTAGRTQSAVAMGWFRQDPGKDRDFVPRTIRWSGGNTYYADSAEGRFTISRDNAKNTVYLQMDSLKPEDTAVYSCAISMNHFGMYDYWGQGTQVTV

SEQ ID NO: 49 - protein, 125 aa, synthetic construct
  REGION 1..125: note = synthetic
QVTLRESGGGLVQAGGSLRLSCKGSGRAINTYAMGWFRQAPGKEREFVPRTISWNGGHTRYADSVQGRFAISRDNADNTMYLQMNSLKPEDTAVYHCPRTYSDYHRIATMEADADSWGQGTQVTV

SEQ ID NO: 50 - protein, 122 aa, synthetic construct
  REGION 1..122: note = synthetic
EVQLVQSGGGLVQAGGSLRLSCPRTSGRTVSNYPMAWFRQAPGKEREFVAHISWSGITSILNSVNDRFTISRDNAKNTIYLQMNSLKPEDTAVYYCPRTAQRPTAGPKGPFGYWGQGTQVTV

SEQ ID NO: 51 - protein, 126 aa, synthetic construct
  REGION 1..126: note = synthetic
EVQLVQSGGGLVQAGGSLRLSCRASGRAINSYNMGWFRQAPGKEREFVSAINWNGARTYYQDALKGRFAISRDNARNTMYLQMNNLKPEDTAVYYCPRTAGRWSPRTVPSGEDQYNFWGQGTQVTV

SEQ ID NO: 52 - protein, 124 aa, synthetic construct
  REGION 1..124: note = synthetic
QVQLVQSGGGLVQAGGSLTVSCTASGRAINMYAMGWFRQAPGKEREFVPRTINWNGAYTQYADSVKGRFTISRDNAKNTMYLQMNSLKPEDTAQYYCSADADYNTYVSPNKRVSYWGQGTQVTV

SEQ ID NO: 53 - protein, 159 aa, synthetic construct
  REGION 1..159: note = synthetic
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNEDRT

SEQ ID NO: 54 - protein, 20 aa, synthetic construct
  REGION 1..20: note = synthetic
GGGGSESKYGPPGGSGGSDP

SEQ ID NO: 55 - protein, 240 aa, synthetic construct
  REGION 1..240: note = synthetic
EIVLTQSPGTLSLSPGERATLSCRASQSVSSSYLAWYQQKPGQAPRLLIYGASSRATGIPDRFSGSGSGTDFTLTISRLEPEDFAVYYCQRSGGSSFTFGPGTKVDIKGGGGSGGGGSGGGGSEVQLVQSGAEVKKPGESLKISCKGSGYNFTSYWIGWVRQMPGKGLELMGIIYPGDSDTRYSPSFQGQVTISADKSISTAYLQWSSLKASDTAMYYCGSGSYFYFDLWGRGTLVTVSS

SEQ ID NO: 56 - protein, 419 aa, synthetic construct
  REGION 1..419: note = synthetic
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNEDRTGGGGSESKYGPPGGSGGSDPEIVLTQSPGTLSLSPGERATLSCRASQSVSSSYLAWYQQKPGQAPRLLIYGASSRATGIPDRFSGSGSGTDFTLTISRLEPEDFAVYYCQRSGGSSFTFGPGTKVDIKGGGGSGGGGSGGGGSEVQLVQSGAEVKKPGESLKISCKGSGYNFTSYWIGWVRQMPGKGLELMGIIYPGDSDTRYSPSFQGQVTISADKSISTAYLQWSSLKASDTAMYYCGSGSYFYFDLWGRGTLVTVSS

SEQ ID NO: 57 - protein, 851 aa, synthetic construct
  REGION 1..851: note = synthetic
MALPVTALLLPLALLLHPRTRPEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITSGGGSDPGGGGSGGGGSPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 58 - protein, 829 aa, synthetic construct
  REGION 1..829: note = synthetic
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITSGGGSDPGGGGSGGGGSPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 59 - protein, 120 aa, synthetic construct
  REGION 1..120: note = synthetic
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 60 - protein, 107 aa, synthetic construct
  REGION 1..107: note = synthetic
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 61 - protein, 245 aa, synthetic construct
  REGION 1..245: note = synthetic
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 62 - protein, 242 aa, synthetic construct
  REGION 1..242: note = synthetic
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 63 - protein, 8 aa, synthetic construct
  REGION 1..8: note = synthetic
GGGGSPAG

SEQ ID NO: 64 - protein, 19 aa, synthetic construct
  REGION 1..19: note = synthetic
SGGGSGGSGGGSGGSDPAG

SEQ ID NO: 65 - protein, 55 aa, synthetic construct
  REGION 1..55: note = synthetic
RVKFSRSADAPAYKQGQNQLYNELNLGRREEYDVLDKRRGRDPEMGGKPRRKNPQ

SEQ ID NO: 66 - protein, 51 aa, synthetic construct
  REGION 1..51: note = synthetic
DKRRGRDPEMGGKPRRKNPQEGLYNELQKDKMAEAYSEIGMKGERRRGKGH

SEQ ID NO: 67 - protein, 38 aa, synthetic construct
  REGION 1..38: note = synthetic
GMKGERRRGKGHDGLYQGLSTATKDTYDALHMQALPPR

SEQ ID NO: 68 - protein, 118 aa, synthetic construct
  REGION 1..118: note = synthetic
QVKLEESGGELVQPGGPLRLSCPRTSGNIFSINRMGWYRQAPGKQRAFVASITVRGITNYADSVKGRFTISVDKSKNTIYLQMNALKPEDTAVYYCNAVSSNRDPDYWGQGTQVTVSS

SEQ ID NO: 69 - protein, 120 aa, synthetic construct
  REGION 1..120: note = synthetic
EVQLVESGGGLVQAGGSLRLSCPRTSGITFSINTMGWYRQAPGKQRELVALISSIGDTYYADSVKGRFTISRDNAKNTVYLQMNSLKPEDTAVYYCKRFRTPRTQGTDYWGQGTLVTVSS

SEQ ID NO: 70 - protein, 324 aa, synthetic construct
  REGION 1..324: note = synthetic
MALPVTALLLPLALLLHPRTRPEVQLVESGGGLVQAGGSLRLSCPRTSGITFSINTMGWYRQAPGKQRELVALISSIGDTYYADSVKGRFTISRDNAKNTVYLQMNSLKPEDTAVYYCKRFRTPRTQGTDYWGQGTLVTVSSTTTPAPRPPTPAPTIASQPLSLRPEACRPPRTGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 71 - protein, 302 aa, synthetic construct
  REGION 1..302: note = synthetic
EVQLVESGGGLVQAGGSLRLSCPRTSGITFSINTMGWYRQAPGKQRELVALISSIGDTYYADSVKGRFTISRDNAKNTVYLQMNSLKPEDTAVYYCKRFRTPRTQGTDYWGQGTLVTVSSTTTPAPRPPTPAPTIASQPLSLRPEACRPPRTGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 125 aa, synthetic construct
  REGION 1..125: note = synthetic
EVQLVESGGSLVQPGGSLRLSCPRTSGFTFDDYAMSWVRQVPGKGLEWVSSINWSGTHTDYADSVKGRFTISRNNANNTLYLQMNSLKSEDTAVYYCAKNWRDAGTTWFEKSGSAGQGTQVTVSS

SEQ ID NO: 73 - protein, 329 aa, synthetic construct
  REGION 1..329: note = synthetic
MALPVTALLLPLALLLHPRTRPEVQLVESGGSLVQPGGSLRLSCPRTSGFTFDDYAMSWVRQVPGKGLEWVSSINWSGTHTDYADSVKGRFTISRNNANNTLYLQMNSLKSEDTAVYYCAKNWRDAGTTWFEKSGSAGQGTQVTVSSTTTPAPRPPTPAPTIASQPLSLRPEACRPPRTGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 74 - protein, 307 aa, synthetic construct
  REGION 1..307: note = synthetic
EVQLVESGGSLVQPGGSLRLSCPRTSGFTFDDYAMSWVRQVPGKGLEWVSSINWSGTHTDYADSVKGRFTISRNNANNTLYLQMNSLKSEDTAVYYCAKNWRDAGTTWFEKSGSAGQGTQVTVSSTTTPAPRPPTPAPTIASQPLSLRPEACRPPRTGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - protein, 121 aa, synthetic construct
  REGION 1..121: note = synthetic
QLQLVESGGGLVQPGGSLRLSCPRTSGVDISSDVMAWYRQAPGKQREFVSGLTRGGSINYADSVKGRFTISRDFAKNTVDLQMNSLKPEDTAVYYCNAEIYTGTFYPRSYWGQGTQVTVSS

SEQ ID NO: 76 - protein, 325 aa, synthetic construct
  REGION 1..325: note = synthetic
MALPVTALLLPLALLLHPRTRPQLQLVESGGGLVQPGGSLRLSCPRTSGVDISSDVMAWYRQAPGKQREFVSGLTRGGSINYADSVKGRFTISRDFAKNTVDLQMNSLKPEDTAVYYCNAEIYTGTFYPRSYWGQGTQVTVSSTTTPAPRPPTPAPTIASQPLSLRPEACRPPRTGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 77 - protein, 303 aa, synthetic construct
  REGION 1..303: note = synthetic
QLQLVESGGGLVQPGGSLRLSCPRTSGVDISSDVMAWYRQAPGKQREFVSGLTRGGSINYADSVKGRFTISRDFAKNTVDLQMNSLKPEDTAVYYCNAEIYTGTFYPRSYWGQGTQVTVSSTTTPAPRPPTPAPTIASQPLSLRPEACRPPRTGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - protein, 122 aa, synthetic construct
  REGION 1..122: note = synthetic
QLQLVESGGGLVQPGGSLRLSCPRTSGGIFSIGVMGWYRQAPGKQRELVATITSRGSTNYADSVKGRFTISGDNAKNTVYLQMNNLKPEDTAVYYCYADLIRPGDFYGMDYWGKGTLVTVSS

SEQ ID NO: 79 - protein, 326 aa, synthetic construct
  REGION 1..326: note = synthetic
MALPVTALLLPLALLLHPRTRPQLQLVESGGGLVQPGGSLRLSCPRTSGGIFSIGVMGWYRQAPGKQRELVATITSRGSTNYADSVKGRFTISGDNAKNTVYLQMNNLKPEDTAVYYCYADLIRPGDFYGMDYWGKGTLVTVSSTTTPAPRPPTPAPTIASQPLSLRPEACRPPRTGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - protein, 304 aa, synthetic construct
  REGION 1..304: note = synthetic
QLQLVESGGGLVQPGGSLRLSCPRTSGGIFSIGVMGWYRQAPGKQRELVATITSRGSTNYADSVKGRFTISGDNAKNTVYLQMNNLKPEDTAVYYCYADLIRPGDFYGMDYWGKGTLVTVSSTTTPAPRPPTPAPTIASQPLSLRPEACRPPRTGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - protein, 117 aa, synthetic construct
  REGION 1..117: note = synthetic
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSS

SEQ ID NO: 82 - protein, 321 aa, synthetic construct
  REGION 1..321: note = synthetic
MALPVTALLLPLALLLHPRTRPQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSTTTPAPRPPTPAPTIASQPLSLRPEACRPPRTGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 83 - protein, 299 aa, synthetic construct
  REGION 1..299: note = synthetic
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSTTTPAPRPPTPAPTIASQPLSLRPEACRPPRTGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - protein, 118 aa, synthetic construct
  REGION 1..118: note = synthetic
QVQLVQSGGGLVHPGGSLRLSCPRTSGIDLSLYRMRWYRQAPGKERDLVALITDDGTSYYEDSVKGRFTITRDNPSNKVFLQMNSLKPEDTAVYYCNAETPLSPVNYWGQGTQVTVSS

SEQ ID NO: 85 - protein, 322 aa, synthetic construct
  REGION 1..322: note = synthetic
MALPVTALLLPLALLLHPRTRPQVQLVQSGGGLVHPGGSLRLSCPRTSGIDLSLYRMRWYRQAPGKERDLVALITDDGTSYYEDSVKGRFTITRDNPSNKVFLQMNSLKPEDTAVYYCNAETPLSPVNYWGQGTQVTVSSTTTPAPRPPTPAPTIASQPLSLRPEACRPPRTGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - protein, 300 aa, synthetic construct
  REGION 1..300: note = synthetic
QVQLVQSGGGLVHPGGSLRLSCPRTSGIDLSLYRMRWYRQAPGKERDLVALITDDGTSYYEDSVKGRFTITRDNPSNKVFLQMNSLKPEDTAVYYCNAETPLSPVNYWGQGTQVTVSSTTTPAPRPPTPAPTIASQPLSLRPEACRPPRTGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - protein, 112 aa, synthetic construct
  REGION 1..112: note = synthetic
EVQLVESGGGLVQPGGSLTLSCPRTSRFMISEYHMHWVRQAPGKGLEWVSDINPAGTTDYAESVKGRFTISRDNAKNTLYLQMNSLKPEDTAVYYCDSYGYRGQGTQVTVSS

SEQ ID NO: 88 - protein, 316 aa, synthetic construct
  REGION 1..316: note = synthetic
MALPVTALLLPLALLLHPRTRPEVQLVESGGGLVQPGGSLTLSCPRTSRFMISEYHMHWVRQAPGKGLEWVSDINPAGTTDYAESVKGRFTISRDNAKNTLYLQMNSLKPEDTAVYYCDSYGYRGQGTQVTVSSTTTPAPRPPTPAPTIASQPLSLRPEACRPPRTGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - protein, 294 aa, synthetic construct
  REGION 1..294: note = synthetic
EVQLVESGGGLVQPGGSLTLSCPRTSRFMISEYHMHWVRQAPGKGLEWVSDINPAGTTDYAESVKGRFTISRDNAKNTLYLQMNSLKPEDTAVYYCDSYGYRGQGTQVTVSSTTTPAPRPPTPAPTIASQPLSLRPEACRPPRTGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 90 - protein, 112 aa, synthetic construct
  REGION 1..112: note = synthetic
EVQLVESGGGLVQPGGSLRLSCPRTSRFMISEYSMHWVRQAPGKGLEWVSTINPAGTTDYAESVKGRFTISRDNAKNTLYLQMNSLRAEDTAVYYCDGYGYRGQGTLVTVSS

SEQ ID NO: 91 - protein, 316 aa, synthetic construct
  REGION 1..316: note = synthetic
MALPVTALLLPLALLLHPRTRPEVQLVESGGGLVQPGGSLRLSCPRTSRFMISEYSMHWVRQAPGKGLEWVSTINPAGTTDYAESVKGRFTISRDNAKNTLYLQMNSLRAEDTAVYYCDGYGYRGQGTLVTVSSTTTPAPRPPTPAPTIASQPLSLRPEACRPPRTGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 92 - protein, 294 aa, synthetic construct
  REGION 1..294: note = synthetic
EVQLVESGGGLVQPGGSLRLSCPRTSRFMISEYSMHWVRQAPGKGLEWVSTINPAGTTDYAESVKGRFTISRDNAKNTLYLQMNSLRAEDTAVYYCDGYGYRGQGTLVTVSSTTTPAPRPPTPAPTIASQPLSLRPEACRPPRTGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 93 - protein, 117 aa, synthetic construct
  REGION 1..117: note = synthetic
QVQLVQSGGGLVQPGGSLRLSCPRTSYFDFDSYEMSWVRQAPGKGLEWIGSIYHSGSTYYNPSLKSRVTISRDNSKNTLYLQMNTLRAEDTATYYCARVNMDRFDYWGQGTLVTVSS

SEQ ID NO: 94 - protein, 321 aa, synthetic construct
  REGION 1..321: note = synthetic
MALPVTALLLPLALLLHPRTRPQVQLVQSGGGLVQPGGSLRLSCPRTSYFDFDSYEMSWVRQAPGKGLEWIGSIYHSGSTYYNPSLKSRVTISRDNSKNTLYLQMNTLRAEDTATYYCARVNMDRFDYWGQGTLVTVSSTTTPAPRPPTPAPTIASQPLSLRPEACRPPRTGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 95 - protein, 299 aa, synthetic construct
  REGION 1..299: note = synthetic
QVQLVQSGGGLVQPGGSLRLSCPRTSYFDFDSYEMSWVRQAPGKGLEWIGSIYHSGSTYYNPSLKSRVTISRDNSKNTLYLQMNTLRAEDTATYYCARVNMDRFDYWGQGTLVTVSSTTTPAPRPPTPAPTIASQPLSLRPEACRPPRTGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 96 - protein, 127 aa, synthetic construct
  REGION 1..127: note = synthetic
QVKLEESGGGSVQTGGSLRLTCPRTSGRTSRSYGMGWFRQAPGKEREFVSGISWRGDSTGYADSVKGRFTISRDNAKNTVDLQMNSLKPEDTAIYYCPRTPRTGSAWYGTLYEYDYWGQGTQVTVSS

SEQ ID NO: 97 - protein, 331 aa, synthetic construct
  REGION 1..331: note = synthetic
MALPVTALLLPLALLLHPRTRPQVKLEESGGGSVQTGGSLRLTCPRTSGRTSRSYGMGWFRQAPGKEREFVSGISWRGDSTGYADSVKGRFTISRDNAKNTVDLQMNSLKPEDTAIYYCPRTPRTGSAWYGTLYEYDYWGQGTQVTVSSTTTPAPRPPTPAPTIASQPLSLRPEACRPPRTGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 98 - protein, 309 aa, synthetic construct
  REGION 1..309: note = synthetic
QVKLEESGGGSVQTGGSLRLTCPRTSGRTSRSYGMGWFRQAPGKEREFVSGISWRGDSTGYADSVKGRFTISRDNAKNTVDLQMNSLKPEDTAIYYCPRTPRTGSAWYGTLYEYDYWGQGTQVTVSSTTTPAPRPPTPAPTIASQPLSLRPEACRPPRTGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 99 - protein, 725 aa, synthetic construct
  REGION 1..725: note = synthetic
MALPVTALLLPLALLLHPRTRPEVQLVESGGGLVQAGGSLRLSCPRTSGITFSINTMGWYRQAPGKQRELVALISSIGDTYYADSVKGRFTISRDNAKNTVYLQMNSLKPEDTAVYYCKRFRTPRTQGTDYWGQGTLVTVSSSGGGSGGSGGGSGGSDPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 100 - protein, 703 aa, synthetic construct
  REGION 1..703: note = synthetic
EVQLVESGGGLVQAGGSLRLSCPRTSGITFSINTMGWYRQAPGKQRELVALISSIGDTYYADSVKGRFTISRDNAKNTVYLQMNSLKPEDTAVYYCKRFRTPRTQGTDYWGQGTLVTVSSSGGGSGGSGGGSGGSDPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 101 - protein, 730 aa, synthetic construct
  REGION 1..730: note = synthetic
MALPVTALLLPLALLLHPRTRPEVQLVESGGSLVQPGGSLRLSCPRTSGFTFDDYAMSWVRQVPGKGLEWVSSINWSGTHTDYADSVKGRFTISRNNANNTLYLQMNSLKSEDTAVYYCAKNWRDAGTTWFEKSGSAGQGTQVTVSSSGGGSGGSGGGSGGSDPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 102 - protein, 708 aa, synthetic construct
  REGION 1..708: note = synthetic
EVQLVESGGSLVQPGGSLRLSCPRTSGFTFDDYAMSWVRQVPGKGLEWVSSINWSGTHTDYADSVKGRFTISRNNANNTLYLQMNSLKSEDTAVYYCAKNWRDAGTTWFEKSGSAGQGTQVTVSSSGGGSGGSGGGSGGSDPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 103 - protein, 726 aa, synthetic construct
  REGION 1..726: note = synthetic
MALPVTALLLPLALLLHPRTRPQLQLVESGGGLVQPGGSLRLSCPRTSGVDISSDVMAWYRQAPGKQREFVSGLTRGGSINYADSVKGRFTISRDFAKNTVDLQMNSLKPEDTAVYYCNAEIYTGTFYPRSYWGQGTQVTVSSSGGGSGGSGGGSGGSDPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 104 - protein, 704 aa, synthetic construct
  REGION 1..704: note = synthetic
QLQLVESGGGLVQPGGSLRLSCPRTSGVDISSDVMAWYRQAPGKQREFVSGLTRGGSINYADSVKGRFTISRDFAKNTVDLQMNSLKPEDTAVYYCNAEIYTGTFYPRSYWGQGTQVTVSSSGGGSGGSGGGSGGSDPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 105 - protein, 727 aa, synthetic construct
  REGION 1..727: note = synthetic
MALPVTALLLPLALLLHPRTRPQLQLVESGGGLVQPGGSLRLSCPRTSGGIFSIGVMGWYRQAPGKQRELVATITSRGSTNYADSVKGRFTISGDNAKNTVYLQMNNLKPEDTAVYYCYADLIRPGDFYGMDYWGKGTLVTVSSSGGGSGGSGGGSGGSDPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 106 - protein, 705 aa, synthetic construct
  REGION 1..705: note = synthetic
QLQLVESGGGLVQPGGSLRLSCPRTSGGIFSIGVMGWYRQAPGKQRELVATITSRGSTNYADSVKGRFTISGDNAKNTVYLQMNNLKPEDTAVYYCYADLIRPGDFYGMDYWGKGTLVTVSSSGGGSGGSGGGSGGSDPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 107 - protein, 722 aa, synthetic construct
  REGION 1..722: note = synthetic
MALPVTALLLPLALLLHPRTRPQVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSSGGGSGGSGGGSGGSDPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 108 - protein, 700 aa, synthetic construct
  REGION 1..700: note = synthetic
QVQLVQSGGGLVQAGGSLRLSCAPSGSIFGIRTMDWYRQAPGKERELVARITMDGRVFHADSVKGRFSGSRDGASNAVYLQMNSLKPDDTAVYYCRYSGLTSREDYWGPGTQVTVSSSGGGSGGSGGGSGGSDPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 109 - protein, 723 aa, synthetic construct
  REGION 1..723: note = synthetic
MALPVTALLLPLALLLHPRTRPQVQLVQSGGGLVHPGGSLRLSCPRTSGIDLSLYRMRWYRQAPGKERDLVALITDDGTSYYEDSVKGRFTITRDNPSNKVFLQMNSLKPEDTAVYYCNAETPLSPVNYWGQGTQVTVSSSGGGSGGSGGGSGGSDPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 110 - protein, 701 aa, synthetic construct
  REGION 1..701: note = synthetic
QVQLVQSGGGLVHPGGSLRLSCPRTSGIDLSLYRMRWYRQAPGKERDLVALITDDGTSYYEDSVKGRFTITRDNPSNKVFLQMNSLKPEDTAVYYCNAETPLSPVNYWGQGTQVTVSSSGGGSGGSGGGSGGSDPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 111 - protein, 717 aa, synthetic construct
  REGION 1..717: note = synthetic
MALPVTALLLPLALLLHPRTRPEVQLVESGGGLVQPGGSLTLSCPRTSRFMISEYHMHWVRQAPGKGLEWVSDINPAGTTDYAESVKGRFTISRDNAKNTLYLQMNSLKPEDTAVYYCDSYGYRGQGTQVTVSSSGGGSGGSGGGSGGSDPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 112 - protein, 695 aa, synthetic construct
  REGION 1..695: note = synthetic
EVQLVESGGGLVQPGGSLTLSCPRTSRFMISEYHMHWVRQAPGKGLEWVSDINPAGTTDYAESVKGRFTISRDNAKNTLYLQMNSLKPEDTAVYYCDSYGYRGQGTQVTVSSSGGGSGGSGGGSGGSDPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 113 - protein, 717 aa, synthetic construct
  REGION 1..717: note = synthetic
MALPVTALLLPLALLLHPRTRPEVQLVESGGGLVQPGGSLRLSCPRTSRFMISEYSMHWVRQAPGKGLEWVSTINPAGTTDYAESVKGRFTISRDNAKNTLYLQMNSLRAEDTAVYYCDGYGYRGQGTLVTVSSSGGGSGGSGGGSGGSDPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 114 - protein, 695 aa, synthetic construct
  REGION 1..695: note = synthetic
EVQLVESGGGLVQPGGSLRLSCPRTSRFMISEYSMHWVRQAPGKGLEWVSTINPAGTTDYAESVKGRFTISRDNAKNTLYLQMNSLRAEDTAVYYCDGYGYRGQGTLVTVSSSGGGSGGSGGGSGGSDPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 115 - protein, 722 aa, synthetic construct
  REGION 1..722: note = synthetic
MALPVTALLLPLALLLHPRTRPQVQLVQSGGGLVQPGGSLRLSCPRTSYFDFDSYEMSWVRQAPGKGLEWIGSIYHSGSTYYNPSLKSRVTISRDNSKNTLYLQMNTLRAEDTATYYCARVNMDRFDYWGQGTLVTVSSSGGGSGGSGGGSGGSDPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 116 - protein, 700 aa, synthetic construct
  REGION 1..700: note = synthetic
QVQLVQSGGGLVQPGGSLRLSCPRTSYFDFDSYEMSWVRQAPGKGLEWIGSIYHSGSTYYNPSLKSRVTISRDNSKNTLYLQMNTLRAEDTATYYCARVNMDRFDYWGQGTLVTVSSSGGGSGGSGGGSGGSDPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 117 - protein, 732 aa, synthetic construct
  REGION 1..732: note = synthetic
MALPVTALLLPLALLLHPRTRPQVKLEESGGGSVQTGGSLRLTCPRTSGRTSRSYGMGWFRQAPGKEREFVSGISWRGDSTGYADSVKGRFTISRDNAKNTVDLQMNSLKPEDTAIYYCPRTPRTGSAWYGTLYEYDYWGQGTQVTVSSSGGGSGGSGGGSGGSDPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 118 - protein, 710 aa, synthetic construct
  REGION 1..710: note = synthetic
QVKLEESGGGSVQTGGSLRLTCPRTSGRTSRSYGMGWFRQAPGKEREFVSGISWRGDSTGYADSVKGRFTISRDNAKNTVDLQMNSLKPEDTAIYYCPRTPRTGSAWYGTLYEYDYWGQGTQVTVSSSGGGSGGSGGGSGGSDPAGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPPRTGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDAYRHQALPPR

SEQ ID NO: 119 - protein, 160 aa, synthetic construct
  REGION 1..160: note = synthetic
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 120 - protein, 265 aa, synthetic construct
  REGION 1..265: note = synthetic
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNGSGGGGSGGGGSGGGGSTTTPAPRPPTPAPTIASQPLSLRPEACRPPRTGGAVHTRGLDFACDIYIWAPLAGTCGVLLLSLVITLYC

SEQ ID NO: 121 - protein, 266 aa, synthetic construct
  REGION 1..266: note = synthetic
MALPVTALLLPLALLLHPRTRPYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNEDGSGGGGSGGGGSGGGGSTTTPAPRPPTPAPTIASQPLSLRPEACRPPRTGGAVHTRGLDFACDIYIWAPLAGTCGVLLLSLVITLYC

SEQ ID NO: 122 - protein, 249 aa, synthetic construct
  REGION 1..249: note = synthetic
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGGGGSGGGGSGGGGSTTTPAPRPPTPAPTIASQPLSLRPEACRPPRTGGAVHTRGLDFACDIYIWAPLAGTCGVLLLSLVITLYC

SEQ ID NO: 123 - protein, 231 aa, synthetic construct
  REGION 1..231: note = synthetic
MLLAMVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIGSGGGGSGGGGSGGGGSTTTPAPRPPTPAPTIASQPLSLRPEACRPPRTGGAVHTRGLDFACDIYIWAPLAGTCGVLLLSLVITLYC

SEQ ID NO: 124 - protein, 249 aa, synthetic construct
  REGION 1..249: note = synthetic
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGSGGGGSGGGGSGGGGSTTTPAPRPPTPAPTIASQPLSLRPEACRPPRTGGAVHTRGLDFACDIYIWAPLAGTCGVLLLSLVITLYC

SEQ ID NO: 125 - protein, 416 aa, synthetic construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGMKGERRRGKGHDGLYQGLSTATKDTYDALHMQALPPR

SEQ ID NO: 126 - protein, 395 aa, synthetic construct
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELGMKGERRRGKGHDGLYQGLSTATKDTYDALHMQALPPR